SEQUENCE LISTING (29 sequences)

SEQ ID NO: 1 - DNA, 1725 bp, Homo sapiens
ggcctgtggggctctgctggatctcctcacccagccacctgactggaattcttcgcagagccagggcacctaaaggcaaaaggtacccggtggtgaggacactcaagcagctttatggaaaagcccacatggtgagcaactgaggcctcctaccaacagccaacaagaaaccggggcctttgccaacagccaagaaataagaatcagaaagaaggacacaccaagaaagaaggacacaccattcatagcacagaacacaagtagatggctcccatgaagaatctagcatcatggtatggcagaagcagaatggactcaggaatcatggtgcctacactggaatttcagatcggccactgacacaatggacacattattcaacatttccaaatcttggcattcttatccacaaagtgaagataataattgtcaattcacaggtgattatgatttaaagagattacttttgaagagttcctaacacattcagtcaacatttaatgatgcttcaggcactgtgttcattgctagtgagcgtatgacacacacagccatacggtcacagagctttcaatgaaaagtaatttctttccggcccccggcttggggcgccttccttccccatggcgggacacctggcttcggatttcgccttctcgccccctccaggcggtgggggtgatgggccatggggggcggagccggctgggttgatcctctgacctggctaagcttccaaggccctcctggagggccagatcgggccgggggttgggccaggctctgaggtgtgggggattcccccttgccccccgccgtatgagttatgtggggggatggcgtactgtgggcctcaggttggagtggggctagtgccccaaggcggcttggagacctctcagcctgagagcgaagcaggagtcggggtggagagcaactccaatggggcctccccggaaccctgcaccgtcccccctggtgccgtgaagctggagaaggagaagctagagcaaaacccggagaagtcccaggacatcaaagctctgcagaaagaactcgagcaatttgccaagctcctgaagcagaagaggatcaccctgggatatacacaggccgatgtggggctcatcctgggggttctatttggaaggtgttcagccaaagaccatctgccgcttggctagcttcaagaacatgtgtaagctgcggcccttgctgcagaagtgggtggaggaagctgacaacatgaaaatcttcaggagatatgcaaagcagaaaccctcatgcagcccgaaagagaaagcgaaccagtatcaaacgggaggacctggagaatttgttcctgcagtgcccgaaacccacactgcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctccggccccagggccccattttggtaccccaggctatgggagccctcacttcactgcactgtactcctcagtccctttccctgagggggaagtctttcccccagtctccgtcatcactctgggctctcccatgcattcaaactgaggtgcctgcccttctaggaatggggaacaggggaggggaggagctaggga

SEQ ID NO: 2 - DNA, 2201 bp, Homo sapiens
ctttgacttcaaggcttagactcgtgcttcctggaatgtccttcattcctccaggctggttccaagcttgtgaagaacatgatcgcagccctccagcaaggccgggtgaaaagaaccactgccttagaaccctgcagatgcaactttggggacctgtcctatgatgcctggcctgcgggctcttacgtgtccccgccgcagacttgtcacagtggcactgtggggctctgctggactcatcagtggctccctcacccacagccttcacaactctgactggaattcttcgcagagccagggcacctaaaggcaaaaggtcaatgacgtaagaagactaatgtttccccatagtcaacgagtagacgttagcacaaaatctacctgctccctcttattctaacttttcacccaaatgaacaatattggcggcaaaacagtgaaggctaattaccctccctgctctctctttctatctctctgggtgaatccagctgacatgttgtgaggacactcaagcagctttatggaaaagcccacatggtgagcaactgaggcctcctaccaacagccaacaagaaaccggggcctttgccaacagccaagaaataagaatcagaaagaaggacacaccattcatagcataagaatcagaaagaaggacacaccattcatagcacagaacacaagtagatggctcccatgaagaatctagcatcatggtatggcagaagcagaatggactcaggaatcatggtgcctacactggaatttcagatcggccactgacacaatggacacattattcaacatttccaaatcttggcattcttatccacaaagtgaagataataattgtcaattcacaggtgattatgatttaaagagattacttttgaagagttcctaacacattcagtcaacatttaatgatgcttcaggcactgtgttcattgctagtgagcgtatgacacacacagccatacggtcacagagctttcaatgaaaagtacataattgctcatttcaccaggcccccggcttggggcgccttccttccccatggcgggacacctggcttcggatttcgccttctcgccccctccaggcggtgggggtgatgggccatggggggcggagccgggctgggttgatcctctgacctggctaagcttccaaggccctcctggagggccaggaatcgggccgggggttgggccaggctctgaggtgtgggggattcccccttgccccccgccgtatgagttatgtggggggatggcgtactgtgggcctcaggttggagtggggctagtgccccaaggcggcttggagacctctcagcctgagagcgaagcaggagtcggggtggagagcaactccaatggggcctccccggaaccctgcaccgtcccccctggtgccgtgaagctggagaaggagaagctagagcaaaacccggagaagtcccaggacatcaaagctctgcagaaagaactcgagcaatttgccaagctcctgaagcagaagaggatcaccctgggatatacacaggccgatgtggggctcatcctgggggttctatttggaaggtgttcagccaaaagaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcatgcagcccgaaagagaaagcgaaccagtatcagaacgggaggcaacctggagaatttgttcctgcagtgcccgaaacccacactgcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctccggccccagggccccattttggtaccccaggctatgggagccctcacttcactgcactgtactcctcagtccctttccctgagggggaagtctttcccccagtctccgtcatcactctgggctctcccatgcattcaaactgaggtgcctgcccttctaggaatggggaacaggggaggggaggagctagggaaagagaacctggagtttgtg

SEQ ID NO: 3 - DNA, 1621 bp, Homo sapiens
ccaggctaagtcacaagcttttgttataaggtctcatcacaccatatgcctctctttcaaaaccagcacctgcagaaattgaggacactcaagcagctttatggaaaagcccacatggtgagcaactgaggcctcctaccaacagccaacaagaaaccggggcctttgccaacagccaagaaataagaatcagaaagaaggaggacacaccattcatagcacagaacacaagtagatggctcccatgaagaatctagcatcatggtatggcagaagcagaatggactcaggaatcatggtgcctacactggaatttcagatcggccactgacacaatggacacattattcaacatttccaaatcttggcattcttatccacaaagtgaagataataattgtcaattcacaggtgattatgatttaaagagattacttttgaagagttcctaacacattcagtcaacatttaatgatgcttcaggcactgtgttcattgctagtagcggacacacacagccatacggtcacagagctttcaatgaaaagtaattcttccaggcggccccccatggcgggacacctggcttcggatttcgccttctcgccccctccaggcggtgggggtgatgggccatggggggcggagccgggctgggttgatcctcgctaagcttccaaggccctcctggagggccaggaatcgggccgggggttgggccaggctgggggattcccccttgccccccgccgtatgagttatgtggggggatggcgtactgtgggcctcaggttggagtggggctagtgccccaaggcggcttggagacctctcagcctgagagcgaagcaggagtcggggtggagagcaactccaatggggcctccccggaaccctgcaccgtcccccctggtgccgtgaagctggagaaggagaagctagagcaaaacccggagaagtcccaggacatcaaagctctgcagaaagaactcgagcaatttgccaagctcctgaagcagaagaggatcaccctgggatatacacaggccgatgtggggctcatcctgggggttctatttggaaggtgttcagccaaaagaccatctgccgctttgaggctgcagcttttcaagaacatgtgtaagctgcggctgcgaagtgggtggaggaagctgacaacatgaaaatcttcaggagatatgcaaagcagaaaccctcatgcagcccgaaagagaaagcgaaccatgcatttcgccgaaacccacactgcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctccggccccagggccccattttggtaccccaggctatgggagccccacttccactgtactcctcagtccctttccctgagggggaagtctttcccccagtctccgtcatcactctgggctctcccatgcattcaaactgaggtgcctgccctctaggaatggggaacaggggaggggaggagctaggg

SEQ ID NO: 4 - DNA, 2166 bp, Homo sapiens
tgatggaaactattgatgcagtgtccaaggtggagaaaggtcagagtggatccagaggagccaagagaagacgtccagcatggtgacctgggctcaagtcaaggtccttatttctctgggacagatggctgagttctactagcaaatgccggtgtggtcctgttggcctaggaaaatccccctgactcacccaggctaagtcacaagcttttgttataaggtctcatcacaccatatgcctctctttcaaaaccagcacctgcagaaatcccttcagcacagccacatgtgaagaacatgatcgcagccctccagcaaggccgggtcccccgcacgcgacttgtcacagtggcactgtggggctctgctggactcatcagtggctcctcacccacagccttcacaactctgactggaattcttcgcagagccagggcacctaaaggcaaaaggtctggtgaatccagctgacatgttgtgaggacactcaagcagctttatggaaaagcccacatggtgagcaactgaggcctcctaccaacagccaacaagaaaccggggcctttgccaacagccaagaaataagaatcagaaagaaggacacaccattcaaaatcagaaagaaggacacaccattctaacagaacacaagtagatggctcccatgaagaatctagcatcatggtatggcagaagcagaatggactcaggaatcatggtgcctacactggaatttcagatcggccactgacacaatggacacattattcaacatttccaaatcttggcattcttatccacaaagtgaagataataattgtcaattcacaggtgattatgatttaaagagattacttttgaagagttcctaacacattcagtcaacatttaatgatgcttcaggcactgtgttcattgctagtgagcgtatgacacacacagccatacggtcacagagctttcaatgaaaagtacataattgctcatttcaccaggcccccggcttggggcgccttccttccccatggcgggacacctggcttcggatttcgccttctcgccccctccaggcggtgggggtgatgggccatggggggcggagccgggctgggttgatcctctgacctggctaagcttccaaggccctcctggagggccaggaatcgggccgggggttgggccaggctctgaggtgtgggggattcccccttgccccccgccgtatgagttatgtggggggatggcgtactgtgggcctcaggttggagtggggctagtgccccaaggcggcttggagacctctcagcctgagagcgaagcaggagtcggggtggagagcaactccaatggggcctccccggaaccctgcaccgtcccccctggtgccgtgaagctggagaaggagaagctagagcaaaacccggagaagtcccaggacatcaaagctctgcagaaagaactcgagcaatttgccaagctcctgaagcagaagaggatcaccctgggatatacacaggccgatgtggggctcatcctgggggttctatttggaaggtgttcagccaaaagaccatctgccgctttgaggctctgcgctagcttcaagaacatgtgtaagctgcggcccttgctgcagaagtgggtggaggaagctgacaacaatgaaaatcttcaggagatatgcaaagcagaaaccctcatgcagcccgaaagagaaagcgaaccagtatcagaaccgaggagaggcaacctggagaatttgttcctgcagtgcccgaaacccacactgcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctccggccccagggccccattttggtaccccaggctatgggagccctcacttcactgcactgtactcctcagtccctttccctgagggggaagtctttcccccagtctccgtcatcactctgggctctcccatgcattcaaactgaggtgcctgcccttctaggaatggggaacaggggaggggaggagctagggaaagagaacctggagtttg

SEQ ID NO: 5 - DNA, 4374 bp, Homo sapiens
gtcccagaacaggttgtgtgtgccccttgaggcgttcatccagcactgtttcagagaaatccctatttcaatctattcctatacgttagttattgaaaagcaatagacaatcacaaaaaacaagttgacctttttgtgttccttgagccaaagggccctcatgactgggcctcacaccgaataactcgttacaaaaagagctagggtcccagactgcgccaaagcttcaggagactgctcctcgtctgtgcacagatgagtggccaactctggagcccaggttgttgcttcctagtctggtggtgaatccttcatagtctgaggagaagaagaaaagaaaaagaaagaaagagagaggaagaggaagatgatcagagtggaaatgtggccaggcgggggtacgatctcgactcactgaaacctctgcttcctggcttcaagcatttctcctgcctcagcctcctgagtagctgagattacaggcgcccaccaccaaaaaaaaatttagcaaattcaaccttaaacctgagtgcatggaaactattgatgcagtgtccaaggtggagaaaggtcagagtggatccagaggagccaagagaagacgtccagcatggtgacctgggctcaagtcaaggtccttatttctctgagtgggacagcattggctgagttctactagcaaatgccggagatgtggttcctgttggccttttaactgatgtgatgaaaaagaaaacacatcttcaggaaaatcacccctgactcacccaggctaagtcacaagcttttgttataaggtctcatcacaccatatgcctctctttcaaaaccagcacctgcagaaatgatcgtgcttccggaatgtccttcattcccggtcacaagaacatgatcgcagccctccagcaccggggaaaagaaccactgccttagaaccctgcagatgcaactttgaattactggacatcatagggactgggcatcctgtccctatgatgcaccatgccgcctgcgggccttcttaatatgcagggctcccccccgacttacagggcactgtggggctctgctggactcatcagtggctccctcacccacagccttcacaactctgactggaattcttcgcagagccagggcacctaaaggcaaaaggtacccggtgaacaaacatctcagctataatttagatccatgctttgtccgcatttgcttccgccccccagttgtccgcagtaagctcacccagcttctctatgttaattttcaaccatatatccagctgactctaagactcccactcctaaatgtcctgcagcccctcaccttaaaatttcccttttgtgtattatttctcgtgacagcccccaaaacccaagagtcttctttgatttttttctctgtctcaacatctccaaacctcccccctagccacttccagtcttatcttttatggtgagaaccagctctcagttgaggtccaacctcagagtagcacttaagtgatacgggaagagcaaagaaaccagtctttatgccacttggaatggatttttccattgcccaaaaaatgttcagaaaataatgaccaatgacgtaagaaggactaatgtttccccgcatttaatagcaagagattccacggaaaataaattgatgttaatactgacaacaaagacaaacaatggtgataaatgaaatggcaggtttcaaactcaatttcatcaggaatttcatcaacatcagagcccctgggccacagtcttcaatgtttgacaaaaaggattaaagggcttatgaccttctgtccagcattcatcctgcccccttccctagctgtacctcattatctatcccagtgcctggcctgtgatatcccttattcagcttagcggatttacgagagacgttcaggtcacaaaatctagcctgtctccccatgaaaccacaatgccattgtctgaacagattaaataaagcagagcatcacctcaaacgattttcatggttcaaatctagtaaccttctatttcactttcattttaaaaaaatctaagtaacaatgaccagaaatattttagtcagtctaaagacagatgataaaatgataactgaaaatcattaacagaattagcagaagttattaccctgagaaaggggacttaaggttggaaggtaaacactcaagacaaatactacagaagtaaagatggccccatggagaaactggcagcatggcatgatggaaatggaatagaatcaggtttttgtccggtgtgactttggtgttctgactcagacacatatttgagaggagaaggaggtaaacacttaccttgtgccagacattgcactaggactttattacctgcaacccactgcaaaactgtgaagatttgatatgatcattctcattttatggttgagaaacaagatcttattctacctcttttcacaatccctttaagaaatggacatccaatatttgagggcgaggcaaaaacatggcctaccaactatagctttacaatacccaaatagtattcccttctaatgaataaaataaaggtcttcctctttcttaatctggtgaatccagctgacatgttgtgaggacactcaagcagctttatggaaaagcccacatggtgagcaactgaggcctcctaccaacagccaacaagaaaccggggcctttgccaacagccaagaaataagaatcagaaagaaggacacaccattcatagcaaagaatcagaaagaaggacacaccattcatagcacagaacacaagtagatggctcccatgaagaatctagcatcatggtatggcagaagcagaatggactcaggaatcatggtgcctacactggaatttcagatcggccactgacacaatggacacattattcaacatttccaaatcttggcattcttatccacaaagtgaagataataattgtcaattcacaggtgattatgatttaaagagattacttttgaagagttcctaacacattcagtcaacatttaatgatgcttcaggcactgtgttcattgctagtgagcgtatgacacacacagccatacggtcacagagctttcaatgaaaagtacataattgctcatttcaccaggcccccggcttggggcgccttccttccccatggcgggacacctggcttcggatttcgccttctcgccccctccaggcggtgggggtgatgggccatggggggcggagccgggctgggttgatcctctgacctggctaagcttccaaggccctcctggagggccaggaatcgggccgggggttgggccaggctctgaggtgtgggggattcccccttgccccccgccgtatgagttatgtggggggatggcgtactgtgggcctcaggttggagtggggctagtgccccaaggcggcttggagacctctcagcctgagagcgaagcaggagtcggggtggagagcaactccaatggggcctccccggaaccctgcaccgtcccccctggtgccgtgaagctggagaaggagaagctagagcaaaacccggagaagtcccaggacatcaaagctctgcagaaagaactcgagcaatttgccaagctcctgaagcagaagaggatcaccctgggatatacacaggccgatgtggggctcatcctgggggttctatttggaaggtgttcagccaaaagaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcatgcagcccgaaagagaaagcgaaccagtatcaacgggaggaacctggagaatttgttcctgcagtgcccgaaacccacactgcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctccggccccagggccccattttggtaccccaggctatgggagccctcacttcactgcactgtactcctcagtccctttccctgagggggaagtctttcccccagtctccgtcatcactctgggctctcccatgcattcaaactgaggtgcctgcccttctaggaatggggaacaggggaggggaggagctagggaaagagaacctggagtttgtgaggactctgaaactagatattttctcaagccctatggacaaacacaaatcatggagaaataaagatgcc

SEQ ID NO: 6 - DNA, 3297 bp, Homo sapiens
gctgttgttcttttcttttccatgatatcattatatggacagtttagggtggtctcatggattataaccatttggatatttggttcactaacaatgctgagatcgcgccattgcactccagcctgggcaacaggagtgaaactccatctcaagaaaaacacaaaatacaaaacaaaacaaaacaaaaaaaaggcggggctcgaccccactttgggaggccgagcgggggtcaggagcagaccatggtgaacccgtctctactaaaaatacaataaattagccgggcatggtggcgggcgcctgtagtcccagctacttgggggctgaggcaggagaatcgcttgaacccgggaggcagaggttgcagtgagccaagatcgtccagcctaggtgacagagtgagactccgtctcaaaaaaaaaaaaaaaaaaaataattttttttaaaatatttaggaagatttgtaaagcttatgaaccattgtaaaaccttatttctaaggtctgtaagacagttttatatgtaataatgtgtccatttccaggaattccccaaatactcatacagccattgagcaccccaatccaaaattcagaatgcttcagataagaacatttttgagtgccgatgtgacactttaaaggaaatgttcattggagcattttgcatttcagatttttggattggaaaactcaaccactgtatttctgcacatattccaaaatctgaaaaagtctgaaacctgagaaactctggtcccaagcattttggaaagggatactcaatctgtattaacatagacttctaaattagtgaaaatttgcattgaataaatggaagtaaatatttttgtttaaatattgttaatttagtcatttaaaaattttgtgatgtaacaaaatgttgaatgaaataatgcttctacaacttaaaattatttttcatagtgaacaaaactagttgaatacttctaaatattcatttttgttagtgccatttttccagttttaggctttagggaattgtgacccttttttttttttcgcggtgtgtggcgcaatctcgcccattttttaattttatggagacagggttcccttggttaactcctgacctcaggtgatccaccccgattggcttaagccccgtgcccggcacttttgtttttaaaataatatcttactttcatatgattctttcagactgtggtatggcattctgaagcaagtcttaacagagctaaaaatcacccttacccaattctctgcactgacatatgtacacaaagtaatattttattattctccttcataaatggttctttaattgaacctttgagccttatactatttttaaatttccctttaaaaattgaattacagccaggcatggtggctcatgcctgtaatcccagaactttgggaggccatggcaggaggattgcttgaacccaggagttgtagaccagcctgggcaagatagcgagatcctgcctctgaaaaaatcataataatactttaatttagaaaataccctgaattagaagtgcttcattcctcatttgtgtacagctgttcctctatatacgaaggggattggtaccagaaccactgcataccaaattccacccatactcaagtctcacagtcttccctgcagaatccaagtatacaaaaagtaggcctatcatttatatgggttttgcatcctgacagtactgtattttcaatcctagtttgtttgggaaaaaaaaatttttacggtacagtatcatacctgttcccagcactttggaaggctgaggcggaggactgttttgtggccaggagttcagcggggcccaaaaaactgggcatgtggccctgcctgtggtcccgctactcggggctgtgaggcaggagaattgcttggcctgggaggttgaggctgcagtgagccgcgatcacaccactgcatgccagcctgggcgacagagtgagaccctgtctcagaaaagaaaaaaaaagtaaaaattgcatgtaagttgacccgcactattcaaatttgtggtgttcaaggttcaactgtaatttcctagcagcattttgtgtgtttgagaatctcttgacactcttcaagtaaatccctaaattacaactttgacatcaaaaaagctagacattcctacatttttgcactacaatacataaaaactcccatgctgatcggttgtgggatcgtgcctgtgaataaccactgcactccagcctgggcaacatagtaagtaagaccttgtctcttaaaaaaaatacattctgaagaaagttctacttatgaatacattttatttataacaaactggtgaaaattttagaccaaaccatgtctttctgggttgtagtgattaaaaaatggttaagagaatgttccctatacaaggcatatgttattaaacatgaaatttaggattagttttctctttgaaaattcttttgatatgctgaggttacagtttggaggttacattagagaaagaatatagggaagatggcagtggtaaaacatttccctgctttacatttttatattggcagtatcttggtttcaacttttctattttcagcaagatatttgcaagtttttactttgaagatgtttggtttatagtagtccatgctatactaatgctgttgctgagataattaagttagatactaatatgaaaaagaatatttgtgtaaaatattttgaagttgtaattagtgtatatacaagttgttaatacttacgaaaaaaggaagcattcccacctgtaataattttgtaacactagactatcagtaaattatcaagaataaaaaccttggtttttcttttaaaacaaattcacaagaaaagtgaaattacacaaaatgataaatttctcatctggtgataaagggaaaaagataatgtaaaagcagattgctgctatttgcataataagccttgttgaagaataagattaagttcaaattttatactctttggtggaattagtcaccatttaataatgaaacaactcatatgttttgtaatgtaaaaatactcacgtttatgatgatgggcctaggtactaaattcctagggatacagtgataatgcaaaaaagccccctgcactcccgcctgagagcgagactctgtctcaaaaaaaaagccccccccatatacactgaaatcttagggaaaaataaaagttaactaggcaagaaggtagggagacctttcagccagcctgtcagagcttttgagtagaaatacggtatcagaaatgtgc

SEQ ID NO: 7 - DNA, 4075 bp, Homo sapiens
actccagcctgggtgacagagtgagactccgtgagccaagatcgtgccactgcactccagcctaggtgacaaaaaaaaaaaatatataattaagcattgttttttaaaaagtagtttaggaagatttgtaaagcttatgaaccattgtaaaaccttatttctaaggtctgtaagacagttttatatgtaataatgtgtccatttccaggaattccccaaatactcatacagccattgagcaccccaatccaaaattcagaatgcttcagataagaacatttttgagtgccgatgtgacactttaaaggaaatgttcattggagcattttgcatttcagatttttggattggaaaactcaaccactgtatttctgcacatattccaaaatctgaaaaagtctgaaacctgagaaactctggtcccaagcattttggaaagggtatactcaatctgtattaacatagacttctaaattagtgaaaatttgcattgaattaaaatgaagtaaatatttttgtttaaatattgtttaaaatttagtcatttaaaaattttgtgatgtaacaaaatgttgaatgaaataatgcttctacaacttaaaattatttttcatagtgaacaaaactagttgaatacttctaaatattcatttttgttagtgcacattttatccagttttaggctttagggaattgtgattttttggtctctcgcccaggctggagtgtgtggcgcaatctcagctcactgcacctctgattcctgggttcaagcaattctcatgtctcgcctccctaatagctggtgcccccccccattttttaaatttttaatggagacagggtttcaccatgttggccgtacccaccttggcctcccaaagtgctgggattaggcttaagccaccgcccggcacttttgtttttaaaataatatcttactttcatatgattctttcagactgtggtatggcattctgaagcaagtcttaacagagctaaaaatcacccttacccaattctctgcactgacatatgtacacaaagtaatattttattattctccttcataaatggtttctttaattgaacctttgagccttatactatttttaaatttccctttaaaaattgaattacagccaggcatggtggctcatgcctgtaatcccagaactttgggaggccatggcaggaggattgcttgaacccaggagttgtagaccagcctgggcaagatagcgagatcctgcctctgaaaaaatcataataatactttaatttagaaaataccctgaattagaagtgcttcattcctcatttgtgtacagctgttcctctatatacgaaggggattggtaccagaaccactgcataccaaattccacccatactcaagtctcacagtcttccctgcagaatccaagtatacaaaaagtaggcctatcatttatatgggttttgcatcctgacagtactgtattttcaatcctagtttgtttgggaaaaaaaaatgtttgtatgcctgggtacagtatcatacctgtaatcccagcactttggaaggctgaggcgggaggactgttttgtggccaggagttcaaaatcagcctggtcgacgtagtgagaccccactctacaaagaaaaattagctgggcatgtggcacctgcctgtggtcccagctactcggaggctgtgaggcaggagaattgcttggcctgggaggttgaggctgcagtgagccgcgatcacaccactgcatgccagcctgggcgacagagtgagaccctgtctcagaaaagaaaaaaaaagtaaaaattgctgtaagttgacccgcactattcaaatttgtggtgttcaaggttcaactgtaatttcctagcagcattttgtgtgtttgagaatctcttgacactcttcaagtaaatccctaaattacaactttgacatcaaaaaagctagacattcctacatttttgcactacaatacataaaaactcccatgctgatcggttgtgggatcgtgcctgtgaataaccactgcactccagcctgggcaacatagtaagtaagaccttgtctcttaaaaaaaatacattctgaagaaagttctacttatgaatacattttatttataacaaactggtgaaaattttagaccaaaccatgtctttctgggttgtagtgattaaaaaatggttaagagaatgttccctatacaaggcatatgttattaaacatgaaatttaggattagttttctctttgaaaattcttttgatatgctgaggttacagtttggaggttacattagagaaagaatatagggaagatggcagtggtaaaacatttccctgctttacatttttatattggcagtatcttggtttcaacttttctattttcagcaagatatttgcaagtttttactttgaagatgtttggtttatagtagtccatgctatactaatgctgttgctgagataattaagttagatactaatatgaaaaagaatatttgtgtaaaatattttgaagttgtaattagtgtatatacaagttgttaatacttacgaaaaaaggaagcattcccacctgtaataattttgttaacactagactatcagtaaattatcaagaataaaaaccttggtttttcattgccaccaacagaagtatttacacaagaacaagtgaaattacacaaaatgataatggggagaaatttctcatctggtgataaagggaaaaagataatgtaaaagcagattgctgctatttgcactttgggaatttctccatgaaattttaacccatttaaaataataaaacattgagaagaaacagcctttgaatttggcaccctgggagaagtgaggggcggccccccccaatatacacacaccctgactttaatgagcttattttgctggggactcagccaattcacaataaaatgagaaaaagaggcctgtggtggtaagcaagtatataaaagaagccttccttgaagcaacgatgcttaagctgaaatcttagggaaaaaaaggaaagaagagggaagactctttcagatagaatcgcagtttgtgagtttgcttggggaaaaaaatttaaaaatataaaaaaaaattgttaatgaaattgaacaggtatgtcagcctacctgacattctagcttctttctttgtcagagtgctaacctcggtgatggattatggctgaacaccccaattatactcttgacatatattcaaccgaagcatgtcaagaatgtccttaatattcaaatagccgggaacatcaagaatagacaagggtaattacacagatacaatgaaaaagaactaaagccgcatgaaaataaaaagacaaaaaataagcatagttagggagcatactgaaatgcagtcttttaaaacagctttagaattccttgccgggcggtaaaaatacaaaattgctgggcgtggtgtcgcgtgccgtgcgcaagaagagcaaaacttcgtctcaaaaaaaaaaaaaaatagattttggcattcaacaaacattggctgttattatattagctattaaaaatttatccatattacagggaaggtagcatgtcaaattggttaaaaagaaaaaagggaactgtagataggaacatatttggtgcaagaaaaattcctgtgattactagcttcactgtgagtttattttttcacgtaataaatccaaggttacataatccataactgatatggcagtttaacaggttattaagaactcaaggcttattgctctattctgccatctttggtgagtggctttcatcctcattgtctcagaaaagttatttcacctctaagtattacatctacatttcaagcaggaagaagatgaagcttaagagaccaaatggagcatgtcagtcaaaggtgccttatttaaatctctggaagccccactgtccggaaacccccttgctgtaagaggtggggttttaagcgggacatcccagccctgagtgaagtcagggattagaagaatggatgctaggtaggtaaatggcaaagacatggtaggcca

SEQ ID NO: 8 - DNA, 2103 bp, Homo sapiens
aatgaatttttggaccctaggttttggctcattggctggatcagtgcatgtaagaattcgacagacccaccttggcctcccaatgggattggcttaagcccggccggcacttttgtttttaaaataatatcttactttcatatgattctttcagactgtggtatggcattctgaagcaagtcttaacagagctaaaaatcacccttacccaattctctgcactgacatatgtacacaaagtaatattttattattctccttcataaatggtttctttaattgaacctttgagccttatactatttttaaatttccctttaaaaattgaattacagccaggcatggtggctcatgcctgtaatcccagaactttgggaggccatggcaggaggattgcttgaacccaggagttgtagaccagcctgggcaagatagcgagatcctgcctctgaaaaaatcataataatactttaatttagaaaataccctgaattagaagtgcttcattcctcatttgtgtacagctgttcctctatatacgaaggggattggtaccagaaccactgcataccaaattccacccatactcaagtctcacagtcttccctgcagaatccaagtatacaaaaagtaggcctatcatttatatgggttttgcatcctgacagtactgtattttcaatcctagtttgtttgggaaaaaaaaatttttatgcctgggtacagtatcatacctgtaatcccagcactttggaaggctgaggcgggggactgttttgtggccaggagttcaaatcagcctggtcgacgtagtgagaccccatctctacaaaagaaaaattagctgggcatgtggcacctgcctgtggtcccagctactcggaggctgtgaggcaggagaattgcttggcctgggaggttgaggctgcagtgagccgcgatcacaccactgcatgccagcctgggcgacagagtgagaccctgtctcagaaaagaaaaaaaaaaaaaattggagttgacccgcactattcaaatttgtggtgttcaaggttcaactgtaatttcctagcagcattttgtgtgtttgagaatctcttgacactcttcaagtaaatccctaaattacaactttgacatcaaaaaagctagacattcctacatttttgcactacaatacataaaaactcccatgctgatcggttgtgggatcgtgcctgtgaataaccactgcactccagcctgggcaacatagtaagtaagaccttgtctcttaaaaaaaatacattctgaagaaagttctacttatgaatacattttatttataacaaactggtgaaaattttagaccaaaccatgtctttctgggttgtagtgattaaaaaatggttaagagaatgttccctatacaaggcatatgttattaaacatgaaatttaggattagttttctctttgaaaattcttttgatatgctgaggttacagtttggaggttacattagagaaagaatatagggaagatggcagtggtaaaacatttccctgctttacatttttatattggcagtatcttggtttcaacttttctattttcagcaagatatttgcaagtttttactttgaagatgtttggtttatagtagtccatgctatactaatgctgttgctgagataattaagttagatactaatatgaaaaagaatatttgtgtaaaatattttgaagttgtaattagtgtatatacaagttgttaatacttacgaaaaaaggaagcattcccacctgtaataattttgttaacactagactatcagtaaattatcaagaataaaaacgtttttctgtaatatttgccactgagcaatttttattaaaaaccaaacagaagtatttacacaagaacaagtgaaattacacaaaatgataatggggagaaatttctcatctggtgataaagggaaaagataatgtaaaagcagattgctgctatttgcataataagcaccttgggtctccatgtaaagtatctttttaatagaagcaccttaatatgaaaacagtaaaaatactcacgtttatgatgatgggcctaggtactaaattc

SEQ ID NO: 9 - DNA, 9037 bp, Homo sapiens
gaggatgaaaacaaaaagacaagacacatgacctctgtgggataaaggcacagggaactataggatgcttgtgaaagggcagtgtagagttgacagatgtgtaccaggtgtagaggacagctgatccagactagaacagatcagaagactagaagtcttaaagatgaaactgatgaatctgaattgtggggaaaaaaaatccaaaatgaaactgatggatctgaaaagaaaattcacaatggagatgattatgtcagaatatccaaaaaggagccaggagggcataaggagttggatcctggtcacatacacatctagatggagtttcacctgctgaactgggccaaagtgtaaaactagctgcatttgacttgagttgtatatactgggcctgatcaaccgcctggagagataaccatacccaactatattgatcattgactagagacatcctgagctgacaaccagccatcatttatcatggactgtgctaaaaccttccagatacgtaaatttacagtagcccttaaaagctctgcctaaccttgcctgaatgcaacagcgctgctagacttgtggcgaggaaagagacaatgcaatccgagtgtgttttcagattgaacttgcccattggttttacttggatttctacatgcagaacacaccaggattacctcagtgtgggataagagactgagtggaaagttttggatgaatggaaggaatataaaatgggagtaccaacatatggtgcaattattcttgatgagacacttgaaaatgtactactgttcaggggtacctagcaaaacaaaagctggggatttccaaaaggaaaagtaaataaagaagaagctcctcatgattgtgctgctagagaggtctttgaagaaatgtaaggatgattacattgaacttcgaatcaatgaccagcttgctcgtttgtacatcattccaggaattccaaaagacacaaaatttaacccaaaaactagaagagaaattcggaacattgagtggttctcttattaatgccttgtcatagaaatgatatgacccccaaatccaaacttggtttggcacctaacaaattttttatggccattccctttatcagaccattaagggactggctttctcgaagatttggcgattcggctttctcgaagatttggcgattcctcagacagtgacaatggattttcctcaactggtagcacgccggctaaacccactgtggaaaaattgagtcgaaccaaattccgccacagtcaccagcagttatttcctgacggttctcctggtgaccagtgggtaaagcacaggcaaccactgcagcaaaagccatataataatcattctgaaatgtctgaccttttaaaaggaaagaatcaaagtatgaggggaaatgcagaaaagcagaaaacagtatcaagattcacctaatcaaaagaaaagaacaaatgggcttcagccagcaaagcagcagaattctttgatgaagtgtgaaaagaaacttcaccacgagaacatgcggggacagcccgtggcatgtaatggacattgcaagttccccttttctccagagcctttttgagtttcaagtttgaccataatgctataatgaaaatcttggacctttgatagcagcacatgtattgtaaatgtcccaggatcagagacctgttgaatttgagtgggtgtctcctcaagccttacctttctcaggtgttttaaagaaatgcagggaggcaatgttctgaagacattttctgtttataagagagtagaaagaaacacgagtttgcactgtaaatgcagttataaccttttatacagattaccttttcagtgttcagtacaagtttaagttgctttctttgagggcatttattctgtgtgactgtgggttttattttgtattctggttaagaaaataatgtattgagttactgtcaagtagccaagttaatgggaatgctccatctacctgttacagtgattgcaataatagtatattggagtttttcaaagaaacttaaagtaatgcccaattattaaatgaacacaagagctgagatgtcacattcgtcagcattacaggtgatctattttggtggcattttgtacttctctctgcttctaaccactgaggctctctaatcttcctctggagttttagtgaaaggatttattgagcagcttctggaatataatgtgcatgtccaaaatgaactcagcgcttcaaaaggacaaagtctgtagcctggaggggcttgagtggatgggagctgatgctgtgattttgagctgtggttacatgcagtcagtaaacctgtgagactgctggaggaaatgtagcagacagcatggaggctgggacccagcagctactttgggtcatgtctttactgtcctgcctccaaccctttagtctcgtagacttttgttcttgtggaaatttcttctgtattccagttgtgtaaatatgtatggaaaactgatattactaggttttacgttgcatctccagtattgatctttggaaactgatgttaattaggttccaattcgcaatagtagcagagactgacatgcttttattgacctgctaagcccctggatgatggagcgagaagggaaggggtatgcagtttacccaagtccaaataaagcatttcactaggttacacgttatgtatatgtgttacataacagtaacatacataactcactcctaggaatgtattttgcttcaggatttttaaattcaaataaggatggcagtgcccattttaaggaggatttcagctataactgaggacaatcaccagggcgttaagggggctttagtgttttggctgcatctgacctcattgaaggaatttcttgggagtcgggggtgtttgagtatatgtgtgtgtgtgtaactatcatgtgggttttaaaaatccttttttacagtggtatccacaaaatacttctctgcttataaatgttattgtagaaatctttattctgtgtaggtagcctatttaattgggttccactgaacctcatgggacttttaaaagaagggctatttaaggaattcacttggtagtactttgctttagcttttaaatggcttgattttaaaggagaaattctatttattaatgaaagttcacccttttggtgctaagcaggagaattcacaattaaagcagtgtgtatagaattatgatattcagaatcctgcatcttttaatgtgacaaatttttgaatcctgtgacttttgctttttaatgcattatatgattgtgtttggaagcttttactgttcttttcagaaatacggctgctttgcttgggtctcttgagccatatttatagttggtcccagcattccagacttctggtaaattgttaaaggagtgaaaatgtagtttgcctggcagatgaataattttcactcatttattgtcttctggaaaccatttaacttttactgttttttatttttttggtatgtgtttttttttttttttatttggccagtcttcctggtgcctgtagagttggatttttcagctctcaatactgaagcaaaagagattatagttaccagaagtatggcctctaatggtgttgagccttttcaagaaccaacaaattatctttgtgattacagtagtttaactatggttaaatgttactgctgttgcgctgctttatttaatgcactgtggaacaaaatataagggtactgttggggtgtgattatgataacttcaggcttttagctctcctcaaagtttgaggttaaaaaaatgttaaaagatgtgagttaaagtttatgtgtgtgatgtgacttaactatgcagaaaatatttaagttggatgtacttgttttaggtacatctactatacttattctctcaaacctgggtaatagtttctcagtgttcaggttaccttgagaaaggccatgcttagcgaactggatttctcctttgagaaattttagtatggtttgaggaaatcaagtgataaaggagtatgctgaatatgcaatcatttacttctactgagtgctgcattttaagatgctagatgagaagagtagttggttttgcttcattatgttttatgtctttgagagagatacaggtcttggaattcactttttaaatgttacgttccaaccttatatgttccaaggtgcagtgcactgtgaatcttttatttttaaaaaaatttgtaggtgcttttatgtataactttaatgatttataagatgctaaatgaattgtttttgtacctaaccattgtaatccaaaagataagggtgtggtttacatgcttttcaaaaatttttgttaagaagtagcaaatgaatggtttaggatttcaaatagtgataccctcctgagacatgagcatttggagaggcctgagaacctaatagaactcatatagtgtgagtgaaatttgattaaagatgttggaaaatgaagttggatttttgtattcatgtataaaggaaagtctcccagtgctgtcaaaggaattgtggttactacttgctgttctgagctttgaatatcgttatttttctattaaatatcaattagtaagtatagctaatgaagacatataatgaaagtaattttatgctgtaagattgaattggtacttttccttattaaggctttagtaatttgtatgtaatgtcaaataaaaggaattcaaatctgagtaaataacagcctcctactataggaattagattcttctataaaagtcaccatattgtatcctatagaaaagttaaggtctgagtgcatgtgtgcattaaaaacatttcactacctattctctcatggacttctcatcctttcttttttcatctccaacagttaaatggtaaggagagcaaagttaattataaaaattagacattttgccaagttagttttcttaccacaatacccctgtttttttttttttcggagtgcaatggtgcaatttcggctcaccgcaacctacgcctcccgggttcaagtgattctcctgcctcagcctcccaagtagctgggattacaggcatgcgccaccacgccctgctaattctgtctttttagtagagacggggtttctccatgttggtcagggtggtctctcttggctttttatctttatttttgtaccagtggaccttcctgctccctgagactgttttgattgacatcttttgtgtttctatattttccgaggcagtattttctttgtatgttaatcatagttatagtaaagtcagactcattaacttttcttcctagtaattttgacttggattattttctagcctgggggatggggttcatatgtgttgttgtaactttagattactaaacaaaacaaactgtttttttgtttgaaggtatcctttacatacctgtctgtaggtaagcaagtgttagtaaaaacaattttgtcaaacagtactcctgacagcttggtaaagtctaggtagagtttctgattttgtattatgtcctaagacttgaaagtgcatttgtctagttgccaaaagttctaaaaatgagatggtgggctttctcagcatctttttgcttcctagaattgagttgctcttgaagtctgtactcctgtgtcagaaagatggcagactgcaaatgggcagtacaggctttgccccatgctttcaaagtaggattctggattgcagtttcaaaaaaatatgatagattattgttatagaatcaaagtgtcctgaagggagaagaatagctatagttgctgggcaaagagtgtaagaaagaaactttagagcatttatcaatacttgagtgctttattatgttccaggctagaaatgtgatcattgcatggcctaagtgtagtcattcatatatatatgaatatctatatctatatctatacatatatatgtatcgccattatcgagtgttttctagccctggataaagttaatttttttgaagtttgattgccaagcaatccagtgaatctaaagtaatttttctcactttaaagaccagtgatgaggagtctggtccttcctgtactgatactgtagaactccagtcaaacttcagtgcactgctttcttacaggaagacaactcagaacacacagccataatctgctttggtgtcagtcacatcacagtggttatacatttgtcctgaatggtgattcattcctttccatctaacttccttgtgacaaatagcatttactattgaaatagtcttaaaatagggaaaagagtgaaagttactcttatgcatgggagtgggcttaaagggttgacttccaccagctgagtacaaacaagtgtaaagaaaaactatgataaagtatggaggtcttgagaattgttcataatgaaatctttaatctgggttatcagagtaatgaaatcaccaataaaaacattccatttatcttggatcttctgtggttagcagaggagtatggtctagctatgttagcagcttgaaggcacaacgtaaaaggaatgagctttcacaaggtgcttacgttagctgggtagtattggtcaaaggtactctgaagagtgaatgcagaaatcagttggctgtgtttgtaatcttgtcccacatggaccctaacctttagtagacttcagtcttttagtccagtagaagacaatggccctgaatatgtgttttctgctttttggtggaaggtttcctgcaggtggttgggtggtttttgggttattggctactgttcgttcagtgccatttgaagacctgttgtttcttaatcctttttcttggtgtgactggccaggaaaaagtccattgagtcctttttttctccctggtgtcttacccctctgtagaataatccatattttaaaatttgtttcacatctttatagcaaacctgccacagacttgcaagcagtgattttatctttccttcctgagaaggtgatctcacttttgatgtttgggtcacctgtggacacctgtgctcttttgagtctcaaggcttatagctttcacattggcttgggaacaacagacttgtgtgtggttctagagtgaaatgggcagtgttctgctggtcctcagtctttgagaggtcttaggcaacatccttagtatagtggaatttctgaggtcaaatttaacatttaatatattctccttttagttcattgaatctgaggttctaaatctgtagcttcattttgagcacaagtctgcattgtcattttttaagagattggcaggcatatcttttgtgatacttaggaaaagactatggatggcagcaaatgaaagtaaaggttctcaactgtgaatgaaatttagaaagataattcttcagcagttggttgattagatgggttagtgcttttggaatatataacagtagacttgtattctttatgcactatacatatgatacgcactgacagaaaagcacagaagacatactgctaagagtttctttgtgcatttcactactgttctaggggactgctgtctctttggttccttttaggtttgtgttaggatgttgcacaagtaatcatttaatgttaaatgcaagtgtccaaagaaatgagtcttaaagttctttggtcatatttcactttagagttaggacaggagacagctaacctgacttccattttacaaatattagaaactttttcttcagtaacaaaaacagggaaggacaataaaaccatgaactattttgctttatgtctgtttttaaaaaataattgttttgaagtttgcttattagcttaataccttatagtacttttaaaaaatcatccttggtaattattgacttatcctagatccatgtacaattcagacttttatctgttagctttaataaaaatttaaaaccatctcatttcagaataaaataaacggaattacagttcatgcattagcatttttaagtgaagttctcttaataagattgccttttgtctaaccgaaatagggcatacagattagtttggatgggaaagtggtaagacttgatttttaatactgtgcctatttccaaaagaagacttgatgtggctttttacattatgctttaaagatgttaatggaggatacatagtcttaaaacaatttgtttgggagtaagttttagtcggaaaaaggtgaagatatttatgctgagttatgttaagcaaattattttgagaccttttgctggtagacaaaaactgaggaaatgtttcactaaatatattatggtcaatgaaaaaaccgactttagagtaaatgtgataaccaagtagatttgtgaactttatttctttagtaattatttttatcttatgtgaaatgattcaacctttaatttttgcctagattatagcaatgttttgttttaccttatagggcattttaatgcttggagaaagactagtaagggattctgttcatggtggtatacagttcttggtgctcttttggaaattgtcaaacatttactgatagcagaaaaggaaccccaaaacacctgtagtgttcaccttgttataagaacagaaacatttggaacaggtttcattctgtttctagatttatgttgttgtagttgaacagcaactgtttttttccctcagtgttaacgaaaggataaagactacctgtattgttgggtatgactatcaaaggatttccggtgattcatgtttgtgaattggagtttaaatgtttgcaggattactgtacttccaaagacccactagaatgtcagctgtactctgtactctccactgagaaaatgaacaaaatcctatgtcttaacagttatgctctaactttttgaaagctttttgatgtaaataataggttagaaactaaaagccttttaatcttacaaaatacaatttgaacgatggaaggttgttcagatactggcttctgtaaaaacatcattgtgttagattgtttgtaatgtactatcaataaaattggctgcttgggc

SEQ ID NO: 10 - DNA, 1192 bp, Homo sapiens
caaagattttggtgtggtcccaacaaacaaaggtcagtttcccttggctgtgggagcccaggagttgtttctgtgcactggtcctatgtgccgctatgctgaagacctggccccctgaagggcaggacctgggatcaaaaggttaaaactagacacaaaggacatttaaaagacttaaaaatagcacaaaggtacatttaaaagacttattggatggaacatgatgggctcatttttaatgtccaaagtggaccaagatctcattatgactcagaaaaaggcagttaaactgaagaaaaaactcttttcagttgtggatcgcaatgatgtcagcaaagggacatgatgggaaggaacctgtgaaattgaaatttgtagatttgcttggtgaccatgggaaacatgtcagtcctctgtgggagttgatcaaatggtgcctgggtctgtcagtgtacaccatcccttccattggactggctttgttggaagaaaagctcagttactttgttggaagaaagctcagatatagcaatgagaaataccaaaagtttaaggcagtggaagaaagcctgcgtaaagagctggtggatatgctaggtgatgatggtgtgttcttatatccctcacatcccacagtggcacctaagcatcatgtccctctaacacggccttcaactttgcttacacaggtgtctatttatcattcttggaccatttatatcctgagatgaacagcattggccatacagcatttaaagatctcatctgagaaacccattgatattggtgccaggagcagatcttttgtgacctcttcagcatccagcattagagtgaaactcccctctcaagagtcctcttgtacccagccacaatgcccactgggactgaatgccaaaggactccctttaggcatccaggttgtggctggaccctttaatgatcatctgaccctggctgtggcccagtacttggagaaaacttttgggggctgggtctgtccaggaaagttttaggaggaccttctgcaaggttaatgtgtgtgtgtgtttgtgttcgtgtggtggtgtttctattaattgggtgaaacaagcaccagcagacaagcagagaaacaactggggaatttattgactcatttagttattctttctacttttatttcccttactaaatatttgcttcttgcttt

SEQ ID NO: 11 - DNA, 390 bp, Homo sapiens
ttcttacaaggtgtcttcagtgccctgggttagtgccctgggttgcctgtgacccaatgcccactgggactgaatgccaaaggactccctttaggcatccaggttgtggctggaccctttaatgatcatctgaccctggctgtggcccagtacttggagaaaacttttgggggctgggtctgtccaggaaagttttaggaggaccttctgcaaggttaatgtgtgtgtgtgtttgtgttcgtgtggtggtgtttctattaattgggtgaaaccaagcaccagcagacaagcagagaaacaactggggatttattgactcttactaaaatggtaatatttgcttcttgcttttatgttactggaaaattaggacatgtaaatggataagtg

SEQ ID NO: 12 - DNA, 599 bp, Homo sapiens
tttgcttacacaggtgtcttcagtgccctgggtttgcctgctgttctcctgggtatcaccagggaggctgtagaaaatgcctccttcctctggaagctttgtcccagaggggcaccaacctgatgccaggagctgtcttgtatgagtgtccttcgaccctgttgggaggtctctcccagtcggatgcacaattcagtgccctgggtttgcctgtgacccaatgcccactgggactgaatgccaaaggactccctttaggcatccaggttgtggctggaccctttaatgatcatctgaccctggctgtggcccagtacttggagaaaacttttgggggctgggtctgtccaggaaagttttaggaggaccttctgcaaggttaatgtgtgtgtgtgtttgtgttcgtgtggtggtgtttctattaattgggtgaaaccaagcaccagcagacaagcagagaaacaactggggaatttattgactcatttagttattctttctacttttatttccttctctaacttgtcttactaaaatggtaatatttgcttcttgcttttatgttactggaaaattaggacatgtaaatggataagtgc

SEQ ID NO: 13 - DNA, 372 bp, Homo sapiens
tgacccaatgcccactgggactgaatgccaaaggactccctttaggcatccaggttgtggctggaccctttaatgatcatctgaccctggctgtggcccagtacttggagaaaacttttgggggctgggtctgtccaggaaagttttaggaggaccttctgcaaggttaatgtgtgtgtgtgtttgtgttcgtgtggtggtgtttctattaattgggtgaaaccaagcaccagcagacaagcagagaaacaactggggaatttattgactcatttagttattctttctacttttattccttctctactaaaatggtaatatttgcttcttgcttttatgttactggaaaattaggacatgtaaatggataag

SEQ ID NO: 14 - DNA, 4086 bp, Homo sapiens
ctggaaattaactgcctccattacactgctgagtcacacacaacgcttgttatgttttcatcacttctctccagaatgaaaacatcagatgaaccaacctgcaacaccttggattcagattggaagataaagagaaagtttaaagaatgtggcctataaaggcgggtacctggaaatattaaccgactcacactgtaaaaatgagaccagtttctaaacaatagagattgtcttagtacaacatcaaggttcaagacaaatgttgaactaaaaactttacactccccacccccacttcagacaggtaccagcattggtgaattatgattttccttagtcagaagcactcatttttacccatgtagacccttaggataaatattggttatttaatgtagattataatgggaagctgatttttttagttagtgtcaaactgagctgaagcttcccaatgcgttttgtacagtctgggaaaaactgtgctgcactgcctttgaaggccatcatgctctttgctgctttggagagtccctagcagacacagaaaatgaatggaggcaaagagtgtgacgggggcaaggaaggaggtcttccagctatacaagttcctgtgggttggcagcgtcgtgtggatcaaaatggagtgctttatgtcagcttactgatggaacatgcaagtgtggcttggaatgtctctattcttcccaaggatagatgttaaggcagatgaagatgtcacaaagctatgcatacataaaagaaaaattattgcagtggccacacttcataaaagcatggaagccccacatccttctctggtgctcaccagtcccggaggaggaacaaatgcaactccagtgacttctcggcggccaaagaaactgaatgaaactttcaagttaatgattggatcatcaaatgccatgggaaggctatatgtacaagaactgcctggaagaacaacaagaactccaccctgtctacccccgacagagattgggcagcagtgaacatggacagaaatctccattccgtggcagccatggaggcctgcccagcccagcgtcatcaggttcccagatatatggagatggttcaatctctccaaggactgacccacttggaagtcctgatgttttcacaagaagtaatcctggttttcatggagctcccaattctagtcctattcacctgaataggactcctctttctccaccttcagtaatgctacatgtctcctgtacagtcatcctgtgcaatggctggaaggactaatatacctctttccccaaccttgactacaaagagtccagtaatgaaaaaaccaatgtgtaatttttcaactaatatggaaatccacgagcaatgttccaccacaaaccaccccaaggcccacctccccctcctccaccttcttgtgctcttcagaaaaagccattaacatctgagaaagatccacttggcattcttgaccctattcctagtaaaccagtgaatcagaaccctgttatcattaatccaaccagtttccattcaaatgtccactctcaggtacctatgatgaatgtaagcatgcctcctgctgttgttcctttgccaagtaatctcccattgccaactgtaaaacctggtcacatgaatcatgggagtcatgtacaaagagttcagcattcagcttcaacctccctgtccccttctccagtgacatcccccgtgcacatgatggggactggaattggaaggattgaggcatcgccccaaagatcacgctcatcttccacatcatcagatcatggaaatttcatgatgccacctgtaggaccccaggccacttctagtggtattaaggttccacccaggtcaccaaggtcaacaatagggtccccaaggccatcaatgccatcaagcccttctaccaagtccgatggacatcatcagtacaaggatatccctaacccattaattgctggaataagtaatgtactaaataccccaagcagtgcagcttttcctactgcatctgccggaagtagttctgtaaagagagcctgttgctttaaatcagatcttgaaccagcacaatgctgcctcctttccagcaagtagtttactctcagcagcagccaaagcacagctagcaaatcaaaacaaacttgctggtaacaacagtagcagcagtagcaattctggagctgttgccggcagtggcaacactgaaggacatagcactttaaacaccatgttccctcctactgccaacatgcttctcccaacaggtgaagggcaaagtggtcgagcagcactaagagataagctgatgtctcagcaaaaagacgcattgcggaaaagaaaacaaccacctacgacagtgttgagtttgctcagacagtctcaaatggatagttctgcagttcctaaacctggcctgacttgctaaggaagcagggtcagggttcatttcccatcagttcaatgtctcagttactacagtctatgagttgtcaaagctctcacttgagtagcaatagtaccccgggttgtggggcctcaaatactgctttgccttgctctgctaaccagctgcattttacagatcccagtatgaactctagtgttcttcagaacatacctttaagaggggaagccgtgcactgccacaatgcaaacactaactttgttcacagtaacagtccagtccccaaccaccatcttgcaggtttaataaatcagattcaggctagcgggaactgtgggatgctcagtcagtcgggcatggctttaggaaattccttacatcccaatccacctcagtcaagaatttcaacgtcctccactccagtgataccaaacagcattgttagcagctataatcaaacaagttctgaagcaggtcttcatttctttagtatttataatttatttacagaatgcttttgtggaagattaacagctaagaggatttcatgtgtttccattaacattggactccaaactgcaaaataagccatcgaagcggtaaatgaagtttatctgaaaatccaaaaaggaagttgtacgaagtagagtttgcatcctatatttgtttgagaaaactcaaaaatggatattgcgggcccaaccacccgccatcacaaagacaacatctgttcttcaagatggcgtcatagtcaccactgcagctggaaacccactgcagagtcagcacccattgggagtgattttccttttgttggccaggagcacgcacttcattttccatccaacagcacttcaaacaaccatcttccacaccccttgaaccccagcctcctcagttctctacctatctctttgccagtgaatcaacagcatctcctaaaccagaatatcctccagcttcagcaggagaaggagtctgagatcaaccccaccctttaggttttctcaacacgcaaaatacccctttaccctcattaacacagtctacagtcgctgttaaacaacaatcagatgtttcctccaaatcagcaacagcagcaacttctccaggggtaccagaatctccaggcgttccaaggacagtccacaattccttgcccagctaacaataaccccatggcttgtctgtttcagaacttctcctaaacaaaaataagcactcagcctgggctcacagcacttcctgagaatccaaacactacacttccaccttttcaagtgagttgcaccgaggattgcccatctttggtcaacaggtaagggcccagggtgctccatgcggttgtcatcaccactgcagtcaacagtacaactcagatcagccccattccagctctgagtgccacgggatggtaggctgaggaattcaagaggcgggctagaggaaaaatgtgaacgaaggagaggatatttcaagtcagcaagttgccacacatccaaaaaagggggggcaaagcccagaggggagcgaaacaggtggagtcgaggaaaaaggcccaacaatgtctctacactgccatttctgcctggggacagcacccaatactgttaccaccaagaaatctagaggaaaatttcaggtataactacaaaagaactaatgag

SEQ ID NO: 15 - DNA, 2760 bp, Homo sapiens
ccacctgcaacaccttgattctaagtttaagaatgtggcctataaaggcgggtacctggaaatattaaccgactcacactgtaaaaatgagaccagtttctaaacaataggattgcttaaagacaaattgaactaaaaactttacactcccccccccttcagacaggtaccagcattggtgaattatgattttccttagtcagagcactcatttttacccgaccatccttaggaattaatattggtatttaatgtagattataatgggagctgattttttagtcaactgagctgaagcttcccaatgcgttttgtacagtctgggaaaaactgtgctgcactggccggctcatgcctgtaatataaggatatcatcttattgctgatatcttggagagtccctagcagacagaaaatgaatggaggcaaagagtacggaggggaaaggagaggtcttccagctatacaagttcctgtgggttggcagcgtcgtgtggatcaaaatggagtgcttatgtcagttgcttggagcaggttaaaacatacctgcttactgatggaacatgcaagtgtggcttggaatgtcctcttattcttcccaaggtacaaaactatgctacataaaaaaaatattgtggcccacttccacatccttctctggtgctcaccagtcccggaggaggaacaaatgcaaccggtccttctcgggcgcaactcaaataagtctcatgaaggaattacaaattgaatgtaagaatcctttcaagttaatgattggatcatcaaatgccatgggaaggctatatgtacaagaactgcctggaagccacaacaagaactccaccctgtctacccccgacagagattgggcagcagtgaacatggacagaaatctccattccgtggcagccatggaggcctgcccagcccagcgtcatcaggttcccagatatatgtcaatcctccaaggactgacccacttggaagtcctgatgttttcacaagaagtaatcctggttttcatggagctcccaattctagtcctattccctgaataggactcctctttctccaccttcagtaatgctgtacagtcatcctgtgcaatggctggaaggactaatatacctctttccccaaccttgactacaaagaccagaatgaaaaaccaatgtgtaatttttcaactaatatggaaataccacgagcaatgttccaccacaacccccaaggccccccctcctcccagaaaaagccattaacatctgagaaagatccacttggcattcttgaccctattcctagtaaaccagtgaatcagaaccctgttatcattaatccaaccagtttccattcaaatgtccactctcaggtacctatgatgaatgtaagcatgcctcctgctgttgttcctttgccaagtaatctcccattgccaactgtaaaacctggtcacatgaatcatgggagtctgtacaaagagttcagcattcagcttcaacctccctgtccccttctccagtgacatcccccgtcacatgatggggactggaattggaaggattgaggcatcgccccaaagatcacgctcatctccacatcatcagatcatggaaatttctaggaccccaggccacttctagtggtattaaggttccacccaggtcaccaaggtcaacaatagggtccccaaggccatcaatgccatcaagccctctaccaagtccgatggacatcatcagtacaaggatatccctaacccattaattgctggaataagtaagtactaaatccccaagagtgcagcttttcctactgcatctgcggaagtagtctgtaaagagtcagcctggtttgctgggaatgcctttaaatcagatcttgaaccagcacaatgctgcctcctttccagcaagtagtttactctcagcagcagccaaagcacagctagcaaatcaaaacaaacttgctggtaacaacagtagcagcagtagcaattcggagctgttgccggcagtggcaacactgaaggacatagcactttaaacaccatgttccctcctactgccaacatgcttctcccaacaggtgaagggcaaagtggtcgagcagccaagagataagctgatgtctcagcaaaaagacgcattgcggaaaagaaaacaaccacctacgacagtgttgagtttgctcagacagtctcaaatggatagttctgcagttcctaaacctggacctgacttgctaaggaagcagggtcagggttcatttcccatcagttcaatgtctcatactacagtctatggtgcaaagctctcacttgagtagcaatagtccccgggttgtggggcctcaaatactgctttgccttgctctgctaaccagctgcattttacagatcccagatgaactctagtgttcttcagaacatacctttaagaggggaagccgtgcactgccacaatgcaaacactaactttgttcacagtaacagtccagtccccaaccaccatcttgcaggtttaataaatcagattcaggctagcgggactgtgggatgctcagtcagtcgggcatggctttaggaaattccttacatcccaatccacctcagtcaagaatttcaacgtcctccctccagtgataccaaacagcattgttagcagctataatcaaacaagttctgaagcaggtccttggtctatgccaattaaaactccagcttttgtttgtattgaatgaagtttattgaaccaaaaa

SEQ ID NO: 16 - DNA, 3890 bp, Homo sapiens
gtaaagacatagagcatctggaaattaactgcctccattacactgctgagtcacacacaacgcttgttatgttttcatcacttctctccagaaggggaattttgaagtcatgaatagatgaaacctgcaacaccttggattcagattggaagtaaagagaaagtttaaagaatgtggcctataaaggcgggtacctggaaatattaaccgactcacactgtaaaaatgagaccagtttctaaacaatagagattgctcaacatcaagagcaaatgttgaactaaaaactttacactccccacccccacttcagacaggtaccagcattggtgaattatgattttccttagtcagaagcactcatttttacccatgtagaccatccttaggattaaatattggttatttaatgtagattataatgggagttagtgtcaaactgaggctacgggaaaaacctgatatctttggagagccctagcagacacagaaaatgaatggagcaaagagtgtgacggaggggacaaggaaggaggtcttccagctatacaagttcctgtgggttggcagcgtcgtgtggatcaaaatggagtctttatgtcagcaggttaaaaatatcttactgatggaacatgcaactgaagtattcttcccaaggaattgaaacagagaaccgcagaagatgttaaggcagatgaagatgtcacaaagctatgcatacataaaagaaaaattattgcagtggccacacttcataaaagcatggaagccccacatccttctctggtgctcaccagtcccggaggaggaacaaatgcaactccagtagtaccttctcggatcctttcaagttaatgattggatcatcaaatgccatgggaaggctatatgtacaagaactgcctggaagccaacaacaagaactccccctgtctacccccgacagagattgggcagcagtgaacatggacagaaatctccattccgtggcagccatggaggcctgcccagcccagcgtcatcaggttcccagatatatggagatggttcaatctctccaaggactgaagtccctggttttcatggagctcccaattctagtcctattcacctgaataggactcctctttctccaccttcagtaatgctacatggttctcctgtacagtcatcccaatggctggaaggactaatatacctctttccccaagcacaatgatggaaatacacgagcaatgttccaccacaaaccaccccaaggcccacctccccctcctccaccttcttgtgctcttcagaaaaagccattaacatctgagaaagatccacttggcattcttgaccctattcctagtaaaccagtgaatcagaaccctgttatcattaatccaaccagtttccattcaaatgtccactctcaggtacctatgatgaatgtaagcatgcctcctgctgttgttcctttgccaagtaatctcccattgccaactgtaaaacctggtcacatgaatcatgggagaagtcagcattcagcttcaacctccctgtccccttctccagtgacatcccccgtgcacatgatggggactggaattggaaggattgaggcatcgccccaaagatcacgccatcttccacatcatcaatcatggaaatttcatgatgcccctgcccgctgggtcccaggtcaccaaggtcaacaatagggtccccaaggccatcaatgccatcaagcccttctaccaagtccgatggacatcatcagtacaaggatatccctaacccattaattggaaaaataccccaagcagtgcagcttttcctactgcatctgccggaagtagttctgtaaagagtcagcctggtttgctgggaatgcctttaaatcagatcttgaaccagcacaatgctgcctcctttccagcaagtagtttactctcagcagcagccaagcacagctagcaaatcaaaacaaacttgctggtaacaacagtagcagcagtagcaattctggagctgttgccggcagtggcaacactgaaggacatagcactttaaacaccatgttccctcctactgccaacatgcttctcccaacaggtgaagggcaaagtggtcggcagcactaagagataagctgatgtctcagcaaaaagacgcattgcggaaaagaaaacaacccctacgacagtgttgagtttgctcagacagtctcaaatggatagttctgcagttcctaaacctggacctgacttgctaaggaagcagggtcagggttcatttcccatcagttcaatgtctcagttactacagtctatgagttgtcaaagctctcacttgagtagcaatagtaccccgggttgtggggcctcaaatactgctttgccttgctctgctaaccagctgcattttacagatcccagatgaactctagtgttcttcagaacatacctttaagaggggaagccgtgcactgccacaatgcaaacactaactttgttcacagtaacagtccagtccccaaccaccatcttgcaggtttaataaatcagattcaggctagcgggaactgtgggatgctcagtcagtcgggcatggctttaggaaattccttacatcccaatccacctcagtcaagaatttcaacgtcctccactccagtgataccaaacagcattgttagcagcataatcaaacaagttctgaagcaggtggtttgtgagacatgtccttggtctatgccaatataaaagccatcccctgcacacaaagacaactcttcaagatggcgtcatatgcagagtcagctacccattgggagtgattttccttttgttggccaggagcacgcacttcattttccatccaacagcacttcaaacaaccatcttccacaccccttgaaccccagcctcctcagttctctacctatctctttgccagtgaatcaacagcatctcctaaccagataaatatccccagccttcagcaggagaagtcgctgttaacaatcaggtttcctccaaatcagcaacagcagcaacttctccaggggtaccagaatctccaggcgttccaaggacagtccacaattccttgcccagctaacaataaccccatggcttgtctgtttcagaatcagcctgggcacagccttcctgagaatccaaacactacacttccactttcaagatacccttgtgagttgcaaccgaggattgacccactcttggtcaacagggaaggatgcctcgttgggtggcccagggatgcttccgtatcacctgcagtcaacagtacaactcagatcagccccagctgccaatctctcggaggtgttgatcatgtgggaggaattcaagaggggctcggctgcccaagaatctagaccatgggaaaaatgtgaacgaaggagattatagttgccacaaaacccagaggggagcgaaacaggtggaagtacgaggaatttttagatatcccagtccaacctggggaacagcacccaatactgttaccaccaagaaactgtccggggagttttaaggaagactagagaacactgtggaaagatgtgcacacataaagggacctcgacagagtcggggatttggagagctgctaagcactgcaaagcaagggtagaggaaaatagtttggaaaattctctgctacatccattacaatggagactttaatgccaaaagcgttaatgggtgtgtgccttatagaccaaggtggctggaaaattagtaagagaagacgacgttcacaattcatgtcaacaaagcccaggaagggccgaggttgaagacacggactgtacaccaaatcccacagggtgacagacaaatgagataacagagctcccactaagcgctattaaagga

SEQ ID NO: 17 - DNA, 4028 bp, Homo sapiens
ctggaaattaactgcctccattacactgctgagtcacacacaacgcttgttatgttttcatcacttctctccagaagattttgaagtcatgaaaacatcagatgaaccaacctgcaacaccttggattcgattggaagataaagagaaagtttaaagaatgtggccttaaaggcgggtacctggaaatattaaccgactcacactgtaaaaatgagaccagtttctaaacaatagagattgtcttagtacaacatcaaggttcaagacaaatgttgaactaaaaactttacactccccacccccacttcagacaggtaccagcattggtgaattatgattttccttagtcagaagcactcatttttacccatgtagaccatccttaggaattaaatattggttatttaatgtagattataatgggaagctgatttttttcacaatggctatttcaagtgtcaactgagctgaagcttcccaatgcgttttgtacagtctgggaaaaactgtgctgcctccacttttgaagctctgtaatttgctgatatctttggagagtccctagcagacacagaaaatgaatggagcgagtgtgacgggggacaaggagaggtcatctgtgggttggcagcgtcgtgtggatcaaaatggagtgctttatgtcagtccagtgggtctttgttatcttgcttggaggttgcttggagcaggttaaaacatacctgcttactgatggaacatgcaagtgtggcttggaatgtcctcttattcttcccaaggttattatccatttttggctgtgaaaagacgcagaagatgttaaggcagatgaagatgtcacaaagctatgcatacataaaagaaaaattattgcagtggccacacttcataaaagcatggaaccagtcccggaggaggaacaaatgcaactccagtagtaccttctcgggcagcaacccaagctctcgggagcaactccaagatcagtaagaaataagtctcatgaaggaattacaaattctttcaagttaatgattgatcatcaaatgccatgggaaggctatatgtacaagaactgcctggaagccaacaacaagaactccaccctgtctacccccgacagagattgggcagcatgaacatggacagaaatctccattccgtggcagccatggaggcctgcccagcccagcgtcatcaggttcccagatatatggagatggttcaatctctccaaggactgacccacttggaagtcctgatgttttcacaagaagtaatcctggttttcatggagctcccaattctagtcctattcacctgaataggactcctctttctccaccttcagtgctacggttctcctgtacagtcatcctgtgcaatggctggaaggactaatatacctctttccccaaccttgactacaaagagtccagtaatgaaaaaaccaatgtgtaatttttcaactaatatggaaataccacgagcaatgttccaccacaaaccaccccaaggcccacctccccctcctccaccttcttgtgctcttcagaaaaagccattaacatctgagaaagatccacttggcattcttgaccctattcctagtaaaccagtgaatcagaaccctgttatcattaatccaaccagtttccattcaaatgtccactctcaggtacctatgatgaatgtaagcatgcctcctgctgttgttcctttgccaagtaatctcccattgccaactgtaaaacctggtcacatgaatcatgggagtcatgtacaaagagttcagcattcagcttcaacctccccccctcccccccgtgcacatgatggggactggaattggaaggattgaggcatcgccccaaagatcacgctcatcttccacatcatcagatcatggaaatttcatgatgccacctgtaggaccccaggccacttctagtggtattaaggttccacccaggtcaccaaggtcaacaatagggtccccaaggccatcaatgccatcaaggatgacatcatcagtacaaggatatccctaacccattaattgctggaataagtaatgtactaaataccccagctgcagcttttcctactgcatctgccggaagtagttctgtaaagagtcagcctggttgcgggaatgcctttaaatcagatcttgaaccagcacaatgctgcctcctttccagcaagtagtttactctcagcagcagccaaagcacagctagcaaatcaaaacaaacttgctggtaacaacagtagcagcagtagcaattctggagctgttgccggcagtggcaacactgaaggacatagcactttaaacaccatgttccctcctactgccaacatgcttctcccaacaggtgaagggcaaagtggcgagcagcactaagagataagctgatgtctcagcaaaaagacgcattgcggaaaagaaaacaaccacctacgacagtgttgagtttgctcagacagtctcaatggatagttctgcagttcctaaacctggacctgacttgctaaggaagcagggtcagggttcatttcccatcagttcaatgtctcagcagtcatgagttgtcaaagctctcacttgagtagcaatagtaccccgggttgtggggcctcaaatactgctttgccttgctctgctaaccagctgcattttacagatcccagtatgaactctagtgttcttagaacatacctttaagaggggaagccgtgcactgccacaatgcaaacactaactttgttcacagtaacagtccagtccccaaccaccatcttgcaggtttaataaatcagattcaggctagcgggaactgtgggatgctcagtcagtcgggcatggctttaggaaattccttacatcccaatccacctcagtcaagaatttcaacgtcctccactccagtgataccaaacagcattgttagcagctataatcaaacaagttctgaagcaggaaaaaaaaatttagaatttcttagtatttataattttatttacagaatgctagggatttgtctattgggaagattaacagcggatttcatgtgtttccattatttgttgtcaatctggttttataaccattggactccaaactgcaaaatacggggggtttcctggtttgtgagacatgtccttggtctatgccaatataaaagccatctcccagcttttgtttgtattcagggaaaaatccaaaaagaaatggattttttttttctagaggaagaaaatgtgttcttatcctagtttgctatccttattatttgtttgagccctgccatcacaaagacaattaagatggcgtcagaacccactgagagtcagctacccattgggagtgattttccttttgttggccaggagcacgcacttcattttccatccaacagcacttcaaacaaccatcttccacactctctacctatctctttgccagtgaatcaacagcatctcctaaaccagaatctattaaacaccgctaacatccagaggtttccatagcaacctcctcccaggcaaccacaccgtaaccatcaactgactcatctacagtcgctgttcctccaaatcagcaacagcagcaacttctccaggggtaccagaatctccaggcgttccaaggacagtccacaattccttgcccagctaacaataaccccatggcttgtctgtttcagaactttcaggcagccgggctcacagcacttcctggaatccaaacactaccttccccttttcaagaaagaacagaaaagtggactaaataaccatttagaagctgctattcatggagtgtctgggactgtacaccaaatcccacagggtgacagacaaatgagaccccccaaacccaagaggaggaagatctccagataacagagactactccactaatgcgcagtgttattaaaggatatgcagatagctaccaccaccacaggggcaatttttttgatcaggatgtgatatcagaaatgtaaatattgtacag

SEQ ID NO: 18 - DNA, 1613 bp, Homo sapiens
aaggatcaagtgtgtgagagggtctctcctgagggaaagggaaaagttaaaaaaaaaacacaaggattttctttgaccttttgggttactctgggcttcatctagccccatactagcctctgagactgcctcagaccgtctgaacattcatggcttccttgggccaagtgtagctgtccagtgtaaaggagtcatagtctggagtgtagaccagggatcttacaaaagcctccttgtccttaggctctgggtagtaggaagccaggttgtgtttgtacgcgtagtcgagaactttgatggcaattctcaaagacacgtctcggatggtgctgagtggtggtggcaattctcaaagacacgtctcggatggtgctgagtggtggtagagtctcccctgggacagatgctgctcagagacttcctgggcaatttgctctgctgtcaggaggaagatctcatctgggatgtgccggctgctgtcaggaggaagatctcatctgggatgtgccggatcccgccggcgatgactcccagtgccaccccggggaacacgtaagcattgtttccctgcccaggaatgaaggtcttgccatcttccagagtcacactcttaaaaggacttccactggcaaaaatccctcggccctcggtgcccgggcaccccctcactcctcggtgacccggtagcacttctcagccgtgcactcggccttgctggtggggttgctcagggcaaagatgatagggcgctcgtggaaggaggccatgtccctcagaatctgctccgtgaaggctcctgcgatggcagcaacacctatgatggctgtgggcttcaccagcctcgatggctgtgggcttcaccagcctcacacctcctccagggagttgacttcaggatggtcttgggcaaacatctccttttcatggttcaggtggctcctccccttgacaatgagccctttagagtccaccatccagatctttctagccctttagagtccaccatccagatctttcttgtggcctctgccttcggtacaccttctttctctagggccatgacaaggaggtgggcaatgcccatagctgcctcgcctgcaccttggaaaacaaacacgtgaatctcgcctgcaccttggaaaacaaacacgtgattggaaagcttgttttggtgattcgcagagcagccaagatccctgccacagcaacggaggctgtgcctcttgtcagggtccccaagtacctccaggtggccaaattcagtaatcagttctcagccttcttctttcttgacctctcagcagcatttggtactgttgcccatttttttcctttgggaagctatttctgcacttggtacaggccagaactcaagaggaaaaacagtggttcttaagctccaaattacaaaatttggcaaataaagttatctccatcattaacctgcatttttggggtgctcttgggctatgcactaggggtttaaaactggaaaggaagctacttagctgtttcgaggtaaccctctgagaagcccatgggtccagggatgtgctggaacatgacttgtgcccataaatgccatgggcagctgaaagtgtggcaggttgttc

SEQ ID NO: 19 - DNA, 2029 bp, Homo sapiens
aagtttaaaaaaaaaacagaggatttttgttctctggcttcatctagccccatctgcctctgagactgcctcaaccgctgaacattcatggcttccttgggccaagtgtagctgtccagtgtaaaggagtcatagtctggagtgtagaccagggatcttacaaaagcctccttgtccttaggctctgggtagtaggaagccaggttgtgtttgtacgcgtagtcgagaactttgatggcaattctcaaagacacgtctcggatggtgctgagtggtggagattctcaaagacacgtctcggatggtgctgagtggtggtagagtctcccctgggacagatgctgctcagagacttcctgggcaatttgctctgctgtcaggaggaagatctcatctgggatgtgccggctgctgtcaggaggaagatctcatctgggatgtgccggatcccgccggcgatgactcccagtgccaccccggggaacacgtaagcattgtttccctgcccaggaatgaaggtcttgccatcttccagagtcacactcttaaaaggacttccactggcaaaaatccctcggccctcggtgacccggtagcacttctcagcctgcccgctgatcggtgacccggtagcacttctcagccgtgcactcggccttgctggtggggttgctcagggcaaagatgatagggcgctcgtggaaggaggccatgtccctcagaatctgctccgtgaaggctcctgcgatggcagcaacacctatgatggctgtgggcttcaccagcctcacacctcctctgatggctgtgggcttcaccagcctcacacctcctccagggagttgacttcaggatggtcttgggcaaacatctccttttcatggttcaggtggctcctccccttgacaatgagccctttagagtccaccccactttccatgagccctttagagtccaccatccagatctttcttgtggcctctgccttcggtacaccttctttctctagggccatgacaaggaggtgggcaatgcccatagctgcctcgcctgcaccttggaaaacaaacacgtgattggaaagtctccctgcacttggaaaacaaacacgtgattggaaagcttgttttggtgattcgcagagcagccaagatccctgccacagcaacggaggctgtgccttcttctttcttgacctctcagcagcatttggtactgttgcccatttttttcctttgggaagctatttctgcacttggtacaggccagaactcaagaggaaaaacagtggttcttaagcccaaattacaaaatttggcaaataaagttatctccatcattaacctgcatttttggggtgctcttgggctatgcactaggggtttaaaactggaaaggaagctacttagctttgaggtaaccactcaaccatgggtccagggatgtgctggaacatgacttgtgcccataaatgccatgggcagctgaaagtgtggcaggttgttcctcatcattgaacatgcagtacttgttacggtatttgttgagcaggcggaaggcattggcattggcgaagtcttcaaattggatgaggcaatttattccaaacttgtctgtcacagcctgcatgaactcatccagcaagtcatcgatgaactcatccagcaagtcatcgtatgccttcccgtgcacgcgctggtgtttcaggccgatgtacagagggtctctgagcagctcctcattgttggtgccgacgtccagcagcacagggaggcactgctgcgtgttggtgccgacgtccagcagcacagggaggcactgctgcgggttccccctcccatgccgtgtacagggccagcttgcccacagggatgcccatgccgtaagcccaggtctcccaggcccaggatgcgctccccatcagtcaccaccacggcctgcatcaaccgcatgaggtggatcgatgatttgtcttgcaagaattcagcattgttgcaagatgacctttgtcatgaatggtgatgaacag

SEQ ID NO: 20 - DNA, 1868 bp, Homo sapiens
gttctctgggcttcatctagccccatactagcctctgagactgcctcagaccgtctgaacattcatggcttccttgggccaagtgtagctgtccagtgtaaaggagtcatagtctggagtgtagaccagggatcttacaaaagcctccttgtccttaggctctgggtagtaggaagccaggttgtgtttgtacgcgtagtcgagaactttgatggcaattctcaaagacacgtctcggatggtgctgagtggtgatggcaattctcaaagacacgtctcggatggtgctgagtggtggtagagtctcccctgggacagatgctgctcagagacttcctgggcaatttgctctgctgtcaggaggaagatctcatctgggatgtgccgggtcaggaggaagatctcatctgggatgtgccggatcccgccggcgatgactcccagtgccaccccggggaacacgtaagcattgtttccctgcccaggaatgaaggtcttgccatcttccagagtcacactcttaaaaggacttccactggcaaaaatccctcggccctcggtgccggccggtagcacttctcagccgtgcactcggccttgctggtggggttgctcagggcaaagatgatagggcgctcgtggaaggaggccatgtccctcagaatctgctccgtgaaggctcctgcgatggcagcaacacctatgatggctgtgggcttcaccagcctcacctgtgggcttcaccagcctcaccacctcctccagggagttgacttcaggatggtcttgggcaaacatctccttttcatggttcaggtggctcctccccttgacaatgagccctttagagtccaccatccagatctttccaaagccctttagagtccaccatccagatctttcttgtggcctctgccttcggtacaccttctttctctagggccatgacaaggaggtgggcaatgcccatagctgcctcgcctgcaccttggaaaacaaacacgtgattggaaactcgcctgcaccttggaaaacaaacacgtgattggaaagcttgttttggtgattcgcagagcagccaagatccctgccacagcaacggaggctgtgcagtacctccaggtggccaaattcagtaatcagttctcagccttcttctttcttgacctctcagcagcatttggtactgttgcccatttttttcctttgggaagctatttctgcacttggtacaggccagaactcaagaggaaaaacagtggttcttaagctccaaattacaaaatttggcaaataaagttatctccatcattaacctgcatttttggggtgctcttgggctatgcactaggggtttaaaactggaaaggaagctacttaggtttcgaggaaccctctgagaagcccatgggtccagggatgtgctggaacatgacttgtgcccataaatgccatgggcagctgaaagtgtggcaggttgttccttgaacatgcagtacttgttacggtatttgttgagcaggcggaaggcattggcattggcgaagtcttcaaattggatgaggcaatttattccaaacttgtctgtcacagcctgcatgaactcatccccagcaagtcatcgtatgccttcccgtgcacgcgctggtgtttcaggccgatgtacagagggtctctgagcagctcctcattgttggtgccgacgtccagcagcacgggcgcgtccagcagcacagggaggcactgctgcgggttcacccctccgcatgccgtgtacagggccagcttgcccacagggatgcccatgccgtagcagcccaggtctcccaggcccaggatgcgctccccatcagtcaccaccacggcct

SEQ ID NO: 21 - DNA, 2645 bp, Homo sapiens
cagaaagttaaaagaaaaaaagtttcttttgacctttatttggtgttactctgggcttcatctagccccatactagcctctgagactgcctcagaccgtctgaacattcatggcttccttgggccaagtgtagctgtccagtgtaaaggagtcatagtctggagtgtagaccagggatcttacaaaagcctccttgtccttaggctctgggtagtaggaagccaggttgtgtttgtacgcgtagtcgagaactttgatggcaattctcaaagacacgtctcggatggtgctgagtggtggtgatggcaattctcaaagacacgtctcggatggtgctgagtggtggtagagtctcccctgggacagatgctgctcagagacttcctgggcaatttgctctgctgtcaggaggaagatctcatctgggatgtgccggatccgctctgctgtcaggaggaagatctcatctgggatgtgccggatcccgccggcgatgactcccagtgccaccccggggaacacgtaagcattgtttccctgcccaggaatgaaggtcttgccatcttccagagtcacactcttaaaaggacttccactggcaaaaatccctcggccctggtccggagctactcattatttttatccggggaaggaagacccgcgacgtgacccggtagcacttctcagccgtgcactcggccttgctggtggggttgctcagggcaaagatgatagggcgctcgtggaaggaggccatgtccctcagaatctgctccgtgaaggctcctgcgatggcagcaacacctatgatggctgtgggcttcaccagccccctcctcctgatggctgtgggcttcaccagcctcacacctcctccagggagttgacttcaggatggtcttgggcaaacatctccttttcatggttcaggtggctcctccccttgacaatgagccctttagagtccaccatccagatctttcttcaatagccctttagagtccaccatccagatctttcttgtggcctctgccttcggtacaccttctttctctagggccatgacaaggaggtgggcaatgcccatagctgcctcgcctgcaccttggaaaacaaacacgtgattggaaagcttgtctcgcctgcaccttggaaaacaaacacgtgattggaaagcttgttttggtgattcgcagagcagccaagatccctgccacagcaacggaggctgtgcctcctcttaccatttcactacagaatttttgcttcctgtaaaatgtcggggacattgtctacccctcttgtcagggtccccaagtacctccaggtggccaaattcagtaatcagttctcagccttcttctttcttgacctctcagcagcatttggtactgttgcccatttttttcctttgggaagctatttctgcacttggtacaggccagaactcaagaggaaaaacagtggttcttaagctccaaattacaaaatttggcaaataaagttatctccatcattaacctgcatttttggggtgctcttgggctatgcactaggggtttaaaactggaaaggaagctacttagctgtttcggtaaccactctgagaagcccatgggtccagggatgtgctggaacatgacttgtgcccataaatgccatgggcagctgaaagtgtggcaggttgttcaaagttgtgacctctttgggcggagaagatctatgtcatcattgaacatgcagtacttgttacggtatttgttgagcaggcggaaggcattggcattggcgaagtcttcaaattggatgaggcaatttattccaaacttgtctgtcacagcctgcatgaactcatccagcaagtcatctgtcacagcctgcatgaactcatccagcaagtcatcgtatgccttcccgtgcacgcgctggtgtttcaggccgatgtacagagggtctctgagcagctcctcattgttggtgccgacgtccagcagcacagggaggcactgctgcgttgttggtgccgacgtccagcagcacagggaggcactgctgcgggttcacccctccgcatgccgtgtacagggccagcttgcccacagggatgcccatgccgtagcagcccaggtctcccaggcccaggatgcgctccccatcagtcaccaccacggcctcaaattaatctatgacttggcccgcatgaggtggatacgatgatgtgctgcccagatcctccttcgaattgtcttctgccaagaattcagcattgttgcaagatgacctttgtcatgaatggtgatgaacagtccacggggcctgcggaaagtcaggccatagtgctgacaggcagcccggaagcaggccatagtgctgacaggcagccccacggtaggcgtgtacacgattggcatgaacttctccactccgaagtcagcactcggtagaagagcttctcgttcggtcttggagtgtcatgagaatgatgtacttgtccaggtcactctgctgccgctgtaatatctctgctgccgctcgtaatatctcatgattcggaggagctggacgtcctggctcagaaagcagggcgggattaggccgtggattccaagctgcagcctttcttcaagggtaaaggccatcccctt

SEQ ID NO: 22 - DNA, 2655 bp, Homo sapiens
attggtgaagaggggtatggctctgatcaagaagggagtttttcagagccaaacacagtggcctgctagtagacagtcaataaatttttttacttaagtgaatgaatttcagactttttttcttgggggattttgtgccatgcaattgttatatcccaggcatcaatgatactcacactgacgaaaatgcctttatgatgagatattgaatgtaaccatgaaagtcactaaatctttctgcatcattgtacatctccctgatgttttctgttttgatgataaccatagtgtctgggcttctcagaagaagatgctgaatggctttgtggacattgaggcccttcggataaaaacatcaatgggaaagggtctgaaatgctggcccagggaaataacaatgacagtatttttttctcctccagttctgtcaatggcccgggtgaggtactccatctctttgactgaataggtcattgatcctatcaagggataacaattttttgccactggatgttgatgttcctatccaaatccacagcaagctggtgttgcaattttccagattcatgcagatccactgacttcagtgcttccatacttcagttttcttttccaaacactctggccaagcattgtttctaagtagcatagcccagcccatgccctgatgcaaagtctacctcacttctgagacagatgagaaaactgtggtttaagctggacaatttaatatcttaatggttccatcaatagtttttctactgggtacatactgaagtgtgattctgcagagtagggtgtaattgctttccttccagttagatagctgtattgtttttttaaaggaagaaactgaaaaatttttgaagaatatgaaaaaaaaggaatttatagcttagactgaggcatactgttgatagttattccatccactggcggatcgtggaatctcccattaggtatatgagttttcctctcaggcattccttcattttgactgtagccaaactacaggagacaggattccatgtgtttctccagacatgcccactggggattgtggatgtcattccaaacttgcatttctctttcattgcaactgtttcttaagtaatcgtcagtatgtattaagagcctctatcggggaaaagtattactgaagtttcctaaataatgttcattaatgttcgttataagagtatatgtttcttaattcttgcataggaatggaagcttttcttcccacaccatcaaaaataaatcctttgaaaaagaaaaaattaacactatggtgaccaccaaatgaatactatgcggaggtttgggccaaagatgaaccagtcactggtttagaataaactgacactcagatggcaggttggaaacctctatggtgatgggcggtgatggggtggtatgaagccatgattgaatattcctcacctggtaactggtgtctgccaaccttagataagccaatttatgaatttttccataatctctacacccacatttgacctttcaaagaggtcagtatataggccaaatcaattcgaatacctttcaaagagtcttttcttgtttgctaagataagaaactttcttgttcttagaatacatgtgagtgagtgcagcacagggcatgtgttgaggcctcacacagtagaagccttcttggtctctgttgtccaggtactggcacaattcagcatttgtgtttaggatcaggccacattcagagtggacttgggaagtgccattgacaaactggccagtgaagatcaccctgtcatagccttggttccttgcactccagagagctgacaccccttcactggggtggatgagcagcagagacagagagacctggccctcccagaacagagtgaagctgaccaggtaggtgccgttgttgaagtcagtcacctttcctgaagcacctgccatcagcgctggggaagacatcctggccctcaggaaatccccgccatattgcttcctgcgtcccaagtggtccctcacctccagcaggatgtgcagctggtctcccctgcagtacgtatctcgagggttgaggatggtggctgtgctatgtgtggcgctggtggtggtgttcacgtgggtgaaaggtctgggtgggatctgctgatctagtttctctatgatttcctttattctgagttcagtctctgttagtggctttaatgatatcagtggtgttttagggaataaggactttgtggagttgttccagtaatggagggagatggataagtttagagcagaccaaacctttgtggagttctggaaaacgtatgtgggtcgaaaaaaatgatccagggctaatataaacaacagtgccaatagtgacttataatttatcatacttattttcatgattaggatcctttagtttacagattgtaacccatacatagactctctttttacagattgttcaatcccatacatagactctctggcgagctaggtgttgagaatttcttctgaagccaccagtgtgctactggcatacctcttgtcaccaggagagccaatggtgtaggttcagttcaaaagctggtaggttgaaggtccaggaagagccaatgttttggttctactgagctctttaccagcgggtcagacttttgt

SEQ ID NO: 23 - DNA, 10591 bp, Homo sapiens
cttgacttcccattggtgaagaggggtatggctctgatcaagaagggtaggctcttttcatgagtatttttagttttatttttaagtgacccagattagaaacatctcatttagctgaattggtggcttatggatcagccataatgtagattctctgctcttgctagttgggaattcagagccaaacacagcatctggcctgctagtagacagtcaataaatttttttttaagtgaatattagcttttttttttaaattataatatattaattatttcctgggggattatttgtgccatatgcaattgttatatcccaggcatcaatgatactcacactgagatcctggaaaatgtcctttatgatgagatattgaatgtaaccatgaaagtcactaaatctttctgcatcattgtacatctccctgatgttttctgttttgatgataaccatagtgtctgggcttctcagaagaagatgctgaatggctttgtggacattgaggcccttcggataaaaacatcaatgggaaagggtctgaaatgctggcccagggaaataacaatgacagtatttttttctcctccagttctgtcaatggcccgggtgaggtactccatctctttgactgaataggtcattgatcctatcaagggataacaattttttgccactggatgttgatgttcctatccaaatccacagcaagctggtgttgcaattttccagattcatgcagatccactgacttcagtgaaagatgtgagtgagatttgattggtataattaaataagctaatcatgtctaatttattataggtttagatgaggggatgtttttgaaaattatttgaacagtcaaaagtgttacgtagcatcttggattaagatggtggataggaggcaggactagcttgcagctctcactcagacaaacagagcagcctgtggagactcacatcatgaacttctgctccaagaactgccacaggaacataccaggaaagtcagaagaatccatagaccctttgaaggaacaggatcaccactgcaggctctctgagatgctgaaaaactgtgtccctagggcaagtttccatcctgcctgtaggacagcagctgatgtgctcttgaaagtgccacctgctggctggaggccaacgaacacaaaaccagcacacaaacaaaaacacaaccaaaacctcacagagtccacttcactcccctgctaactccactggagcaggtgctggtattcaaggctgcaagacctgaagatgggtcacatcctatgggattctttgcagacactctccagtaccagcccagagcccagtagctccactgggttgcgagactcaggaagccccattcctagggaagggggattaaaccacataggagcccctgtgggacaaaagaatctgaatagcagcccttgaatcccagatcttccctctgacatgtctaccaaaatgagaagaaaccccaaaaccaattctggtaatatgacaaaacaagattctttaactcccaaacccaacaaaatataggaaaaataatttagaaggttgattattaagctaatcaaggaggcaccagagaaaggtgaagtccaacttaaattaaaaacatgatacagaatatgaaaggaaaattcttcaaaaaaaaaaaattctggaaatcaaggatacacttagagaaatgcaaaatgcactggaaatctcaaaattgaacaagcagaaaaaagaacctcagagctagaagacaaggctttagaattaactcaatccatcaaagacaaaaaggattttaaaaaaaacaaaccaaagtttggggagtaaatccccaacctaagaataattggtattcccaaggaagaagagaaatttaaaactggaaaacatatttgagggactaatcgaagaaaatttccctggccttgctagagatctagacatccaaatacaagagctcaaagaacacctgggaaattcatccagaaagatctaggcacatagtaatcagattatctaaagtcaagatgaaggaaaaaatcttaagagctgtgggcaaaggatcaggtaacctataaaagaaaacttatcagattaacagctgatttctcagcagaaaccctacaagctagaagggattgggttctattttacccttaaacaaaaaaataatttcaaaataaatgaagaaaagatagtcttttcaaaattccaaaaaaaaagagctctaaatcttgaagcaagttatcaaagcacaccaaaatagaacctccttaaagcataattctcaaagcaaaaagaaaagaaaaacaaggtatttagcaacaagtagcacaaaataatattaaacgatgctctacttaaaactacagaatggaagaatgggtaagaattcaccagccaagtttctgctgtcttcaggaaagtcacctataaaaggggaaaagatattccatgcaaatgggcaccaaaagcaagcaggagtagcaattcttatatcagacaaaacaaacttaaattaaaaaaggtaaaaaaatagaaacacaattctaaatatatataactggagctcccaaatttacaaaaattatatagaccaaaaaaaagaagggattacccacagactggtcatcaagacagaaagtcaacaaagaaataatggacttaaaccaaacaaatgaacttaactttctccaaaaagtgaagaatatacattctatttatcagcacatggaacattctccaagataagtgcaaaaaattaaaaaaatacccacagtggaataaaattggaaatcagctccaaaaggaaccctcaaaaccatgcaaatacatggaaattaaaaaccttgaatgatcattgagtcaacaataaaataaatggaaattaaaaaattctttaaaaccaaaagtgataaatcaaaacctctgggatacagcaaaagtgatgttaagaggaaagttcatagacttaaatgcctacataaaagtctgagagggcacaaatagataaactaatgtcatagctcatggaactggagaaacaagaaaatccaacccaacgaaaaaaaaaaaaaaaaaacaaaaaacaaaaataaatgaaacaaaagtttaaaaaaaaataactataaaaataaaaaggagatattacaacgataccacagaaatatgaaagttagtgaaccctttaacaaaaagaaaacctaagggaaatctggaaatatacaaccctcctagattaaaccaggaagatatagaatctctgaacgtaacaaacagtgagattgaaatgatagttaaaaaattcacaaaaatgtcgtttaaagaaaaattcaaacaaacttcccattccaaaaaagagaaagagggaatcctccctaaataattcttaaaccacacggagaaaaaaaaaaactacagaccagaacatcccttatgaacatagatgctaaaatcctcccaaaatactagctaccaaatccaatggcatatcaaaaagataatccaccatgatcaagtgggtttcatcggcagggggctaaaactaagtcaataaatgtgacaaataaaaaaaaacacagaaaaagcatttgagaaaatccagcatcctagattaaaaccgcaaaatcaacatagaagggaaaccttaaggaacatctaaaaaaccctccttagacaaaaaaaagaagaaataaaggacaccaatgaaagaaatgcaaaccaatcaaaaccccagaacggtaaatgaattagcaaagttttagaaaaataatactgctatacccaacagcaaccaagccgagaatcaaatcaagagctcaaaaaaaaaaataaaccagagtaacaaggaaaactacaaaacaccgcccaaagaaataaaaaagaaacacatgggggaaaaaatttgaaaatcaaaaaatctaaaattcaacaattttttcaaaaatggaacaaaccagaaataaagcccaatacttacagccaactgatctttgacaaagcaaacaaaaacataaagagggtaaaggatgaccatataaaagaatgaaacttcacaatttataaaaaaggctcatatccagaatattcctaaagaactaaaaacaatcattaccacggaggaagggcgtggggggaaaatttatcagaacatcggctaactgctttgtagttagaacttttgaggaaaatgttcagaggaggattattgatgttttaacatggtgtttaagcaaagggctacatttgaagacttaacccaggctggaaaacagagatgctgtagggtgagttggaagaaaatggcagagttattttcttcactctgcttccaacttacttcagttttcttttccaaatcacgcattaatttaaaacagttgttttttaaataaggggtaactccataacacttatatcccaagagaatcactgttacagaatgtggctaaaggctgttactagaccctgaattttgtagatgaggaattgaggtttccctaggagtttagtcatttaccagagacacaatttaaagtcttccttccattgagaatttttcttataacttagaactaacccatttcctttgattctattgaggcctttgagccgggagccacagcactgcaattctctcagtgatgagagccaggatccggggcataagggggacaataatggttttgtaggtgagatggctaaggtctactagcaaattctgagctattactggtgcgctcttggtttagtaaatagagcaactgaactaggattttagcaactgggttttatccctgacccttaaattaagtaatgataacaattgcaatggctgttgtttttttggttcctataattcttttatttttttaaggacgggattataaggaaaggaggcttatttggctgatggttcttgcaggctgtacaagcatggcatcggcatctgttcagcatctggtgaggccgggaagcttttagtcatggcggaagggaaggggagccagtgtgtcacatggtgagagagggagcaagagagatgccaggctcttttaaacaaccagttctcacatgaactgacagagcgagaactcactcattccaggggagggcccaagccagtcatgagagatccccccggcccaaacacttcccgccccgtggattatgggaaatttcgttgtggaacggggaaaataaataacatgcccaaagtcattgtttctaagtagcatagcctagatctgttagaccccaacccatgctttacttgtgtctacctgatgcaaagtctacctcacttctgacatctgttctcttaggatgatgacagaacaaaaaacaaattctaaatctgcattacctcattttactaggtagaaactcttttcatcttcagttgacagatgagaaagaggtttaacttgttcaaagtccccatagaggagagaagtcaggttttgaacccaggtgtctggtttaaacccttaatcatttgttagattatatttatatttgtctcaatagtttttgggctcggatactgaagtgtgaattctgcagagatagggatgtaataaacctttttctacactgctttccttccagttagatagctgtattgtttttttaaagaagaaacaatccttgaaaaattttggtgtgttgtgattgaagaatatgaaaaaaaaggaattcccattcccttctcccctttaatggaaaagaagtttctgaaagggtaagaattatagcttagactgaggcatactgttgatactggctttgaagtattccatccactggcggatcgtggaatctcccattaggtatatgagttttcctctcaggcattccttcattttgactgtagccaaactacaggagacaggattccatgtgtttctccagacatgcccactggggattgtggatgtcattccaaacttgcatttctctttcattgcaactgtttcttaaagtaatccttctcctctatatggggaggggaaaagtattactgaagtattctcttaatggaacagctgtttttttaataaaaatttttctctgatgtgaatttcctaaataatgttcattaatgttcgttataagagtatatgtttcttcccagaaaaaaaatggctttctggaggaaaatttaagtattttccttttctatgaactaaatcacaattcttgctaggaatggaagctcaattatcctatcattttagctcactttacccctaagtttgtcacactgtgtcttaaatgacaccatcaaaatataaatcctttgtaaataaaggataaaaaatattaaaaagtttggcttgggacgccaatccaaatgaatatagctatagctggagaggtcttagaggtccaagatgggccaaagatgaacccctcagtcactggtttagaagagtctgggaatccacaggttgaggtagccacaatggtgaggagcccgtggcaggttgattattaatcaactgagagaagagtttaatattttaataaggggtctggtaaaaataacatttctacatctaggtacatatttacacttcagtgtaaataggtttattagtgattagcaaggcttctttctttttttttaagtttaatgcagagacataaaagctcttgccttgagtcgttttagataagaatttcctaggctatttggattcaaagaaattggagagaaattagtaaatataagtgtgcaagttctattttttatgctaacaattgtttgaaaggtggcaaagatcaagagggaggggttataataaactgacactcagatggcaggttggaaacctctagatggtgatgggctagtggtgatatgggaagaggtgatgggagtggtatgaagagaggtgttgtggtgaggtaaagaatgggtatagaattgggaaagagttaacgtttaaaggttgttgttaaaatgggcagggaattcactgactgacaaatagactgatgatgagagttacagttactaaaataggcttatgatattccagtgattgaattattcactcacctaggtaactggtgtcttggccaaccttagataagccagataacagagtgctgataggtgaaaaataaagaaacctttaaatgggtctagaactaggccccccaaaagagattactttgcatttggagacactaattgtattgaatttttccataatctctacacccacatttgacctctaaaaccaaaattaatacactcacaagacttttctttggatcagtatataggccaaatcacaatatttgcacaggtagtctcaagaagtaattatttttacctttcaaagaggctcttttcttgtttgctaagataagaaactttcttgttcttagaatacatgtgagtgagtgcagcacagggcatgtgttgaggcctcacacagtagaagccttcttggtctctgttgtccaggtactggcacaattcagcatttgtgtttaggatcaggccacattcagagtggacttgggaagtgccattgacaaactggccagtgaagatcaccctgtcatagccttggttccttgcactccagagagctgacaccccttcactggggtggatgagcagcagagacagagagacctggccctcccagaacagagtgaagctgaccaggtaggtgccgttgttgaagtcagtcacctttcctgaagcacctgccatcagcgctggggaagacatcctggccctcaggaaatccccgccatattgcttcctgcgtcccaagtggtccctcacctccagcaggatgtgcagctggtctcccctgcagtacgtatctcgagggttgaggatggtggctgtgctatgtgtggcgctggtggtggtgttcacgtgggtgaaaggtctgggtgggatctgctgatctagtttctctatgatttcctttattctgagttcagtctctgttagtggctttaatgatatcagtggtgttttagggaataaggactttgtggagttgttccagtaatggagggagatggataagtttagagcagaccaaacctttgtggagttctggaaaactgtaaaaatgtctggaaaactgtaaaaatgatccaggaggctaatataaacaacagtgccaatagtgacttataatttatcatacttattttcatgattaggatcctttagtttacagatttcattagtttacagattgttcaatcccatacatagactctctggcgagctaggtgttgagaatttcttctgaagccaccagtgtgctactggcatacctcttgtcgtgtctttatagtagaatggtttatattcctttgggtatatacccagtactgggcttgctgggtcaaatggcatttctagttctagatccttgaggaatcgccacactgtcttccacaatggttgaactaatttacactctcaccaacagtgtaaaataattcctacttctccacatgctctccagcatcggtgtttcctgacattttaatgatcgccattctaactggcatgagatcgtggttttgtggttttgatttgcatttctctaatgaccagtgatgatgagcattttttaatgtttgttggctgcataaatgtcttcttttgagaagtgtccattcatatcctttgcccacattttgatggggttgtttgtttttttcttgtaaatttaagttctttgtagattctggatattagccctttgtcagatggatagattgcaaaaattttctcccattctgtaggtttcctgttcattctgatgatagtttcttttgctgtgcagaagcttttagtttaattagatcccatttgtcaattttgacttttgttgccactgcttttggtgttttagtcatgaagtctttgcccatgcctacatcctgaatggtactgcctaggttttcttctagtgtttttatagctttaggtcttacatttaagtctttaattcatcttgagttactttttgtataaggtgtaaggaaggggtccagtttcagttttctgtatatggctaggcagttttcccaacaccatttattaaataggaatcctttccccattgcttgtttttgtcaggtttgtcaaagatcagatggttgtagatgtgtggcgttatttctgaggcctgtgttctgttccattggtctatatatctgttttggataccagttccatgctgttttcattactgtagccttgtagtatagtttaaagtcaggtagcatgatcctccacctttgttctttttgcttaagattgtcttggctatatgggctcttttttggtaccatatgaaacttaaagtagttttttttttaaatttgtgaagaaagtctgtggtagcttgatggggatagcagtgaatctataaattactttaggcagaatggccactttcatgatattaattcttcctattcatgagcatggagtgtttttccatttttttgtgtcctctcttattttcttgagcagtgatttgtagttccccttgaagaggtccttcatatcccttttaagttgtattgctaggcattttattctctttgcagcaattgtgaatgggagttcactaatgttttggctctctattattggtgtataagaatgcttgggatttttgcacattgattttgtatcctgagactttgctgaagttgcttatcagcttaaggagattttgggctgagatgatggggttttctaaatatacaatcatggcatctgcaaacagagacaatatgacttcctctcttcctgttcgaatacctttatttctttctcttgcctgatttccctggccagaacttccaatactacgttgaataggagtggtaagagaggacatccttgtcttgtgctggttttcaaagggaatgcttccagcttttgcccattcagtatgatattggcctgaatttgaaggcatgagaaccaggagagccaatggtgtaggttcagttcaaaagctggtaggttgaaggtccaggaagagccaatgttttggttctacttaactgggcaggaagaattctctcttactcagcgagggtcagacttttgttctattcaggctttcacaaattggatgaggctcaccaaaataaaaaattaacaagatttggaaggggacaccagaaaagaggggaggggcagcctggagaacaagctgagaggaagaaacgaggaaaaaccctcttgcaactcagcccccaccctaagcacaaggcaacaaagaatttgaagtctgtggtgtggaccctcgcggttgagtgttacagttcttaaagatggtgtgtctggggtttcttccttcagatgttcagatgtgtccggagtttcttccttgcggtgggttcgtggtctcgctgactttaggagtgaagccgcagaccttgttacagctcgttaaggtggcacgtccggagttgtttgttcctcctggtgggttcgtggtctcgtggtctcgctgggagtgaagctgcaaacgtttgcgggagtgttacagctcataaag

SEQ ID NO: 24 - DNA, 4411 bp, Homo sapiens
ttacaactatgggaccaccagcaggagcagctactactccaagttccaggcagggaatggctcatggggatatccgatctacaatggaaccctcaagcgggagcctgacaacaaaggcgcttcagctcctacagccagatggagaactggagccggcactacccccggggcagctgtaacaccaccggcgcaggcagcgacatctgcttcatgcagaaaataaggcgagccgcagtgagcccgacctctactgtgacccacggggcaccctgcgcaagggcacgctgggcagcaagggccagaagaccacccagaaccgcacagcttttacagcacctgcagtggtcagaaggccataaagaagtgccctgtgcgcccgccctcttgtgcctccaagcaggaccctgtgtatatcccgcccatctcctgcaacaaggacctgtcctttggccactctagggccagctccaagatctgcagtgaggacatcgagccatccccaaggcgtgcagtacctgagctcccaggatgagaagtaccaggccattggggcctattacatccagcatacctgcttccaggatgaatctgccaagcaacaggtctatcagctgggaggcctgcaagctggtggacctcctccgcagccccaaccagaacgtccagcaggcgcggcaggggcccgcgcaacctggtgttcaggagcaccaccaacaagctggagacccggaggcagaatgggatccgcgaggcagtcagcctcctgaggagaaccgggaacgccgagatccagaagcagctgactgggctgctctggaacctgtcttccacgggaactcattgccgacgccctgcctgttctggccgaccgcgtcatcattcccttctctggctggtgcgatggcaatagcaacatgtcccgggaagtggtggaccctgaggtcttcttcaatgccacaggctgcttgaggaacctgagctcggccggccgccagaccatgcgtaactactcagggctcattgattccctcatggcctatgtccagaactgtgtagcggccagccgctgtgacgacaagtctgtggaaaactgcttggtgtttgcacaacctctcctaccgcctggacgccgaggtgcccacccgctaccgccagctggagtataacgcccgcaacgcctacaccgagaagtcctccactggctgcttcagcaacaagagcgacaagatgatgaacaacaactatgactgcccctgaggaagagaccaaccccaagggcagcggctggttgtaccattcagatgccatccgcacctacctgaacctcatgggcaagagcaagaaagatgctaccctggaggcctgtgctggtgccctgcagaacctgacagccagcaaggggctgatgtccagtggcatgagccagggtgaaaagggcctgccacaaattgcccgcctcctgcaatctggcaactctgatgtggtgcggtccggagcctccctcctgagcaacatgtcccgccaccctctgctgcacagagtgatggggaaccaggtgttcccggagggaccaggcctcctcaccagccacactggcaataccagcaactccgaagacatcttgtcctcggcctgctacactgtgaggaacctgatggcctcgcagccacaactggccaagcagtacttctccagcagcatgctcaacaacatcatcaacctgtgccgaagcagtgcctcacccaaggccgcagaagctgccccccggcttctcctgtctgacatgtggtccagcaaggaactgcagggtgtcctcagacagtgctgggaaccttagctggggccaacagcctcaggaacttcacctcccgattctaagaagagactgtccaagcaagttaggcttgcaggaagatatgacccagctgagaagccccaggcctcgctggatggggttttctgtccatcctgtgctatttgggaatggatatggaaagatttttagattttttttttgtcttaaaaccacaaacaaaaagctaggtaagctattttgttgcagctcataaggtggtgaaaaggactctcctgtgtttcttactcataggcaaggacaacatgtgctttttggtgagctgctcataattcctgaaatgtgtggtgccagggcaagggggccatcactgcagtcaggccctcagaggagtcctgcaggcttcctaccagtggtctccagggtgcaggagtaactggggctgggccagcctcccccaatgctttccaggaagggaggtctggtgtatctcatgggagaatctggggtgtctgtatgtcacccctccagcagcgccacaaggactgaggttgggtaggtgtggggagcccatttaaggtgctcagaggagtaggagctgaaagatggtgccttccaccctcttgggctgtgtgcccatcagagcaggctcagcctgcaaaggccctgcattcagaggtcttgtaatctacttgttgcaggagaaagaaaaaaaatgttttttaaaagctattttattgcagctctttcccaagagctgttctgggaatggctggtcttcatattcccagtggagaggggaacaagtggggctgggcatatacctattccggcttctagtgggatggagttggggtatagaaattaaccaggaagatgtttccaccaagcctgctgtgagtcaattgagggagtgtttggggtcccaggagacttggacggggggagtttgggtagactaggaaaggaaagtgccatatcagggtaccggtaccggcaagctcacatctcagccaggggccatgccccacttcccctgaccccagctgtcttgtctccactctgtgaaacccacaggggatgtgataaacagggctattaggggtatcagccacgtcgagcccccagactctgtgcacttcagaccagcagcagcaggagggctcccgagggccttatgagaaaacctgtgtggacatcccttggtgtacactaagacagagcagagcccagcgctcccaagccttcctccttccagcttctacctccatgctagcattgctggtgttagagaggaattaacttcctggtctgtgcccttctctagaagaatataagatgctcctcctcctcaccccttctcagcctcctcccaagtcttcctcttctgcaccacccccgagtccaaacccacctcttgccccagcattcaggctggaaaacactgatgtggactcagtatgaagggggagcagggcgtcctccgagagggtccccggctgttagccagctgtgctgtggtgctgtgggtctgtcataccctcccttgcttctgttcacactgggaggcccactcctggctcacctctccctctcagggacccacgtgggagcctggatccctggactgtcctgggcataggtttcagggcctcctttgttgtcatcagaacccagaggaattcttctcctaaaaaatacgtatggcataccaatctgtgcggggcagtgtcctaagcacttagactacatcagggaagaacacagaccacatccctgtcctcatgcggcttatgttttctggaggaaagtggagacacaagtccttggctttagggctcccccggctgggggctgtgcagtccggtcagggcgggaggggaaatgcaccgctgcatgtgaaccttaccagcccaggcggatgccccttccccttagcactaccctggcctcctgcatcccctcgcctcatgttcctcccaccttcaaagaatgaagagccccatgggcccagcccctgccctgggaaccaggcagccttccagacctcaggggctgaggcagactattagggcagggctgactttggtgacactgcccattccctctcaggccagctcaggtcacccgggcctctgacccaggcctgtcactttgagaggggcaaaactgagaggggcttttcctagagaaagagaacaaggagcttgccaggcttcatgtagccgacacacgtctcaggattttaagtccacattggcctcacactaccagggccaatgcccaaaataaggagttccaatttggggccaaatgaggaaggacacagactctgccctgggatctcctgtgctagcggccaatgacaaatccagtcattggccaccagccacctctgcagtggggaccacactagcagccctgactccacactcctcctggggacccaagaggcagtgttgctgtctgctgtccaccttggaatctggctgaactggctggaggaccaagactgcggctgggggggcagggaagggaagccgggggctgctgtgagggatcttggagcttcgtagcccaccttcccttggtttgtag

SEQ ID NO: 25 - DNA, 8862 bp, Homo sapiens
gtgggcgtaggaccctccgagccaggatcaactattgttactccattactggacagatcaaaacaaggagacagaactggattcaaagatgccagatgtgggagacacgccaaagctcccaagaacttttggatgtggggaacatcgggcgcctggagcagcgcatgatcacagtggtgtatgggcctgacctcgtgaacatctcccatttgaatctcgtggctttccaagaagaagtggccaaggaatggacaaatgaggttttcagtttggcaacaaacctgcaaatgaggttttcagtttggcaacaaacctgctggcccaaaacatgtccagggatgcatttctggaaaaagcctatactaaacttaagctgcaagtcactccagaagggcggctgcaagtcactccagaagggcgtattcctctcaaaaacatatatcgcttgttttcagcagatcggaagcgagttgaaactttttgcagatcggaagcgagttgaaactgctttagaggcttgtagtcttccatcttcaaggaaaagtgagcagagctgtggaaaggctcccccaaaacattttcaccttcattttataaagcaaataaaatgcttgaacctgctgctattgcttttttttgaagaaaaatataacaattaggtcctgtccccgcttcaacaaatatttcttgagctcttactatgtgcccataggcattgagaatatggggtgaagaaaatgaaactcaaggtttcaaggacaatagagtcttctggggaaactagccaataagcatgccaacaaatgcgtctgtaaactgggggaagtgctataaaaagaaagactggagaactactattgagaagaagggaagctcattccagaaaaagaaaacaatgctcgcaaatccctgaggctggaaataatttccctactggatttaagcaactgagaaaagaccaccgcattcaagcataaagagaaaacatgagagatggatttgcaaagacatgcaaagaatttttttccttttgcaatcacttctgattttaccttttctggaaaccctggaaatcaaaatattaacttatctagggaaagaaggaatagaaaattaaatgtatgaaccaattccccgtaaattgtaattttctaaacaactgattctgacattgtaactaagttttatatgttcaaactttcagaccaatgtcaatgcaggcaaatttaaaaggaaagaaaaaatgataattatctgaagttggagaaaactttaaaaaacctctatacggatgatagattagaatgctttttaaaaatctaaaaaccaaagactgtttcagagctaggaaataaatgtttctgtgaactgactatgagtatcaaaactctgttttaaatctctgtgataatttcattcctacataacaatgactctagaaaatatccactaattagcaaaaaattttggccatattcacaagaggtattttaaacaagccacggaagaaaagggttatataaatcatagttggttctgctaaatgggagcatattttgtagccaagtcatttaagctagatttaattgtgctctgtaaatagtaccagaaacacagtggatgtcctgttgagatttctttttaaaacatgatattactgcacattgttggggataaataaggcacttgaacttgtatcctggcgacgtgatccactagaataacatattcctttagaagatgctactctttagaaatcacagttgctagcctgaagttaccctaattgttaaaaggtagtactgtatccaaagagagagacaaagcaattactgtccttctgttaacaacccaagcatattttccagtgagacccttgaaaactgttccgtgttcaaaaaattaccactttacccacaaactttctgagaactgaaaatagttatttgcaccaaattcccttgatttaaggcttattaacttgaaaagagctcagtgacattgataaacttgccacacactggcatacggagtgtttaacaaagataaacccatgtatttgtttcaaataataaagtttactgtagacctcaagaagatttcactccagaagtgtacagagttttcctcaacaacctttgccctcgacctgaaattgataacatcttttcagaatttggtgcaaaaagcaaaccatatcttaccgttaaaagcaaaccatatcttaccgttgatcagatgatggattttatcaaccttaagcagcgagatcctcggcttaatgaaatactttatccacctctaaaacaagagcaagtccaagtattgattgagaagtatgaacccaacaacagcctcgccagaaaaggacaaatatcagtggatgggttcatgcgctatctgagtggagaaaatcatggatgggttcatgcgctatctgagtggagaagaaaacggagtcgtttcacctgagaaactggatttgaatgaagacatgtctcagcccctttctcactatttcattaattcctcgcacaacacctacctcacagctggccaactggctggaaactcctctgttgagatgctttagatgtatcgccaagtgctcctgtctggttgtcgctgtgtggagctggactgctggaagggacggactgcagaagaggaacctgtcatcacccatggcttcaccatgacaactgaaatatctttcaaggaagtgatagaagcaattgcggagtgtgcatttaagagcggagtgtgcatttaagacttcaccttttccaattctcctttcgtttgagaaccatgtggattccccaaagcagcaagccaagatggcggagtactgccgactgaaaagcggagtactgccgactgatctttggggatgcccttctcatggagcccctggaaaaatatccactggaatctggagttcctcttccaagcccttggagttcctcttccaagccctatggatttaatgtataaaattttggtgaaaaataagaagaaatcacacaagtcatcagaaggaagcggcaaaaagaagctctcagaacaagcctccaacacctacagtgactcctccagcatgttcgagccctcatccccaggagccggagaagctgatacggaaagtgacgacgacgatgatgatgagagacgacgacgatgatgatgatgactgtaaaaaatcttcaatggatgaggggactgctggaagtgaggctatggccacagaagagctggaagtgaggctatggccacagaagaaatgtctaatctggtgaactatattcagccagtcaagtttgagtcatttgaaatttcaaaaaaaagaaataaaagttttgaaatgtcttccttcgaaagttttgaaatgtcttccttcgtggaaaccaaaggacttgaacaactcaccaagtctccagtggaatttgtagaatataacaaaatgcagcttagcaggatatatccaaaaaaaagcagcttagcaggatatatccaaaaggaacacgtgtggattcatccaactatatgcctcagctcttctggaatgcaggttgtcagatggtggcacttaatttccagacaatggacctggctatgcaaataaatatggggatgtatgaatacaacggtatgcaaataaatatggggatgtatgaatacaacgggaagagtggctacagattgaagccagagttcatgaggaggcctgacaagcattttgatccatttactgaaggcatcgtagatgggatagtggcaaacactttgtctgttaagattatttcaggtcagtttctttctgataagaaagttgggcctttcaggtcagtttctttctgataagaaagttgggacttacgtggaagtagatatgtttggtttgcctgtggatacaaggaggaaggcatttaagaccaaaacatcccaaggaaatgctgtgaatcctgtctgggaagaagaacctattgtgttcaaaaaggtggttcttcctactctggcctgtttgagaatagcagtttattctctggcctgtttgagaatagcagtttatgaagaaggaggtaaattcattggccaccgtatcttgccagtgcaagccattcggccaggctatcactatatctgtctaaggaatgaaaggaaccagcctatcactatatctgtctaaggaatgaaaggaaccagcctctgacgctgcctgctgtctttgtctacatagaagtgaaagactatgtgccagacacatatgcagatgtcatcgaagctttatcaaacccaatccgatatgtgaacctgcagctttatcaaacccaatccgatatgtgaacctgatggaacagagagctaagcaattggctgctttgacactggaagatgaagaagaagtaaagaaagaggctgatcctggagaaacaccatcagagctccaagtgaagcaccatcagaggctccaagtgaagcagaacgactccagcagaaaatggggtgaatcacactacaaccctgacacccaagccaccctcccaggctctccacagccagccagctccaggttctgtaaaggcacctgccaaaacagaagatcttattcagaaaggcacctgccaaaacagaagatcttattcagagtgtcttaacagaagtggaagcacagaccatcgaagaactaaagcaacagaagcacagaccatcgaagaactaaagcaacagaaatcgtttgtgaaacttcaaaagaaacactacaaagaaatgaaagacctggttaagagacaccacaagaaaaccactgaccttatcaaagaacacactaccaagtataatgaaattcagaatgactacttgagaaggagagccgctttggaaaagtccgccaaaaaggacagtaagaaaaaatcggaacccagcagccctgatcatggttcatcaacgattgagctcatggttcatcaacgattgagcaagacctcgctgcctggatgctgaaatgacccaaaagttaatagacttgaaggacaaacaacagcagcagctgcttaatcttcggcaagaacagtattatagtgaaaaataccagaagcgagaacatattaaactgcttattcaaaagttgacggatgtcgcagaagagtgtcagaaccggatgtcgcagaagagtgtcagaacaatcagttaaagaagctcaaagaaatctgtgagaaagaaaagaaagaattaaagaagaaaatggataaaaagaggcaaaaagaataaagaagaaaatggataaaaagaggcaggagaagataacagaagctaaatccaaagacaaaagtcagatggaagaggagaagacagagatgatccggtcatatatccaggaagggtagacagagatgatccggtcatatatccaggaagtggtgcagtatatcaagaggtagaagaagcgcaaagtaaacggcaagaaaaactcgtagaggcgcaaagtaaacggcaagaaaaactcgtagagaaacacaaggaaatacgtcagcagatcctggatgaaaagcccaagcctcttcttgtcggaaacttgccatgaggatccctctgtttcccccaactttactccccccaaccctcaagctctcaagtggtgaccaccgtccttccggccagctgaagaataccaagacaaattcaaaagactgcccctcgagattttggaattcgtgcaggaagccatgaaaggaaagatcagtgaagacagcaatcacggttctgcccctctctccctgtcctcagaccctggaaaagtgaaccacaagactccctccagtgaggagctgggaggagacatcccaggaaaagaatttgatactcctctgtgaatgctcctgccaggccttcagaaattgcatggccactccagcgtcatcggactctctcttattacaaagatcactgcccaggaccatcttcccgagaagcatcccttagcctaaaatccacaccaaagggagagttccagaggaatccatgaagaattcccatgcccaggctccatgtgtcatgtggaaacctccacaggtctgctagtgaagaatgcatgtatgtgagatttttgttttctttccaatagcaaattcaaagcaagcaacttgcaggctccatggaacttttaatgaaggacagtgtcttctttgaagaaaatcaagctcgtgtttttattcgaagctctggtgtaaaatatttcaaagtcatagaaatagtttgagaaatgcatagcattatttaacactattgaacagccgactttgagcattgtttcttctaactgcccctcaactaccattatcttcaagtcaacatgcatattacattttcatcctttgctttgcaagcactggtggcttgcagtttgctaatttatttatcatagagtcatcaatgtatttgttgctgacatggttttattagatactgtagtgattcaaataagttttctatttgaaaaaaaaaaattgtatcttgcccagtgaagccatcctaagacttagcaatatggattgtacatttggctgcatgagcaagtcggccgcacacttccagacagtgtgctgtttgaattgactatttgcactcaaagtctgggtattcattggttattggcctgaaatgatcaaataactacaaatgatctgttgaataaaaatagttgagctgatatatgttaagcagatattcaatcagaatgaacaggttccggtggttattttgccgtttgacattttttatggttcatttatttttaatatagagaggaagattgaatatttatctagagaatacaaagacccacatgtaaatgataggtattatctccatgtatatatgtacccacttagtcatgtaagtgcatatacacatacacacacatgagtgtagacatgtgtttattaattgacaatgacccaaatctcttccacaagacttaaaaccaaattcagggacaaatggatagagaagaaaagggtcaaacatcgagattacatggatgttaaattatatggagacgctaagaaataattgatggagccattgatgcaaaccgaagtagatttagaacttatatgaatttgatttatattttgcaagatcaaaaattagatgttaagtatcagattttaagcttgttttaatggtcaaaaaattaggacagaaataatatggacatttattagtatcttccataatttttaagtctgacacatttctattttattctaacataaaaaaacttccattatatatttactaagtatatttaattcacttaactctgtctttataagttcctattttagatggaataagagaaacaaattatatcaaggtaaaactgatcaaaagcataattgaaagttctgaaaaaagaaaaataataatatgtagaaaaatgtaacttagagagtaacacatgaacattgaagttaaaacccagaagccagatgctcacagttttattttactttaaaataaacctgtctgactgtagctttggaaatatcttaaaacgcaaaaccattaattgaatttaatatttttatgaaaattattttatttaactttaagcaataactagagattacaattaaattttaatcaaaatgaaggcttagttcaaacataaggaaacagtgtttgattaaaaaaaacacatctagtaagacgtaaggggaaaatcacatcctctttggagatgattatattttgatctgaagggtttggggtgttcattagacacttaataaaacttaacttccaatgaaaaagaaatcttttgtaaatcattctacttttgcactttgaaagaaaggcgttaatcataaagaagcaagaatggtcaaaatcgaatgctgcatttttataaacaaaattacagactgtctgaaattgaagaagaatgaactaataatagcattcataaccaaacacaatgatgattattgcagaacattgtatcacattttagtccagagatagaataaagttgaataaccttgacttacacaaaactgttttggtagttggatttcattatcttagtgaatttagtcattttacaatatgtttgtatttggccatttactgtaatcacatttttatatctgtacaatgacactttttgcagttgtggggtagtgtgtaacactgtccatcttgcatcattgaaactactacaatgatactatcatttaataatattaatattacttgaaatagactaagataaagaaaaggggtctgtatgatgtgcagttttgtgcctttatgtatttgccttgttctttgtcgaatgtgtgaaattcgtactgtggtttttcctataatagaaagtagagctgtgtattaaattagactgtgtctctctgatacctttacactactgagaatagcatggttttggccatgtaaaccaattttcaaagttctaatgacatagccatgtgtttttggttttttatatttcattttaaaatttgagtatcaccatacattaattaatactcctgtagtagataagctgtcattaagtaattcccaaaaaaagggccatttgcttgcattactttgaatttaatgttgcgcttgtgcactgtgttaatattgtttgtgatggattggacgttgtgactcttgccttttaagaagaaaaaaaagataggacaaagtatttgaagctctaaaaatattttggttcttctatctcaaattatttaaaatgcataattcacatttttgtaataattctatgcaattttgtggcatgatgtttcttccacttgtaattttatgtgctttcatcacaaatccaaa

SEQ ID NO: 26 - DNA, 5798 bp, Homo sapiens
gcatgatcacagtggtgtatgggcctgacctcgtgaacatctcccatttgaatctcgtggctttccaagaagaagtggccaatcagtttggcaacaaacctgctggcccaaaacatgtccagggatgcatttctggaaaaagcctatactaaacttaagctgcaagtcactccagaagcactccagaagggcgtattcctctcaaaaacatatatcgcttgttttcagcagatcggaagcgagttgaaactgctttagaggcttgtagtcttccatctgtaaacgggggaagtgctataaaaagaaagactggagaactactattgagaagaagggaagctcattccagaaaaaaatctctggatttaagcaactgagaaaagaccaccgcattcaagcataaagagaaaacatgagagaaaagcaattactgtccttctgttaacaacccaagcatattttccagtgagacccttgaaaactgttccgtgtcaaaaaatccttttccagaaggtacagagttttcctcaacaacctttgccctcgacctgaataccgttgatcagatgacaaccttaagcagcgagatcctcggcttaatgaaatactttatccacctctaaaacaagagcaagtccaagtattgattgagaagtatgaacccaacaacagcctcgccagaaaaggacaaatatcagtggatggagaagaaaacggagtcgtttcacctgagaaactggatttgaatgaagacatgtctcagcccctttctcactatttcattaattcctcgcacaacacctacctcacagctggccaactggctggaaactccgctcctgtctggttgtcgctgtgtggagctggactgctggaagggacggactgcagaagaggaacctgtcatcacccatggcttcaccatgacaactgaaatatctttcaaggaagtgatagaagcaattgcggtttccaattctcctttcgtttgagaaccatgtggattccccaactggaatctggagttccttggatttaatgtataaaattttggtgaaaaataagaagaaatcacacaagtcatcagaaggaagcggcaaaaagaagctctcagaacaagcctccaacacctacagtgactcctccagcatgttcgagccctcatccccaggagccggagaagctgatacggaaagtgacggaggctatggccacagaagaaatgtctaatctggtgaactatattcagccagtcaagtttgagtcatttgaaatttcaaaaaaaaaaaaggaacacgtgtggattcatccaactatatgcctcagctcttctggaatgcaggttgtcagatggtggcacttaatttccagacaatggacctggctatgcaaataaatatggggtgaatacaacgggaagagtggctacagattgaagccagagttcatgaggaggcctgacaagcattttgatccatttactgaaggcatcgtagatgggatagtggcaaacactttgtctgttaagattatttcagggaaagttgggacttacgtggaagtagatatgtttggtttgcctgtggatacaaggaggaaggcatttaagaccaaaacatcccaaggaaatgctgtgaatcctgtctgggaagaagaacctattgtgttcaaaaaggtggttcttcctactctggcccctgtttgagaatagcagtttatgaagaaggaggtaaattcattggccaccgtatcttgccagtgcaagccattcggccaggctatcactatatctgtctaaggaatgaaaatgaaaggaaccagcctctgacgctgcctgctgtctttgtctacatagaagtgaaagactatgtgccagacacatatgcagatgtcatcgaagctttatcaaacccaatccgatatgaacctgatggaacagagagctaagcaattggctgctttgacactggaagatgaagaagaagtaaagaaagaggctgatcctggagaaacccaggaagcaagactccagcagaaaatggggtgaatcacactacaaccctgacacccaagccaccctcccaggctctccacagccagccagctccaggttctgtaaaggcacctgccaaaacaatcttattcagagtgtcttaacagaagtggaagcacagaccatcgaagaactaaaatcgaagaactaaagcaacagaaatcgtttgtgaaacttcaaaagaaacactacaaagaaatgaaagacctggttaagagacaccacaagaaaaccactgaccttatcaaagaacacactaccaagtataatgaaattcagaatgactacttgagaaggagagccgctttggaaaagtccgccaaaagaaaaaatcggaacccagcagtgacccaaaagttaatagacttgaaggacaaacaacagcagcagctgcttaatcttcggcaagaacagtattatagtgaaaaataccagaagcgagaacatattaaactgcttattcaaaagttgacgagtgtcagaacaatcagttaaagaagctcaaagaaatctgtgagaaagaaaagaaagaattaaagaagaaaatggatagaagaaaatggataaaaagaggcaggagaagataacagaagctaaatccaaagacaaaagtcagatggaagaggagaagacagagatgatccggtcatatatccaggagagatgatccggtcatatatccaggaagtggtgcagtatatcaagaggtagaagaagcgcaaagtaaacggcagaaaacaaggaaatacgtcagcagatcctggatgaaaagcccaagctctgtttcccccaactttactccccccaaccctcaagctctcaagtggtgaccaccgtccttccggcccccctcgagattttggaattcgtgcaggaagccatgaaaggaaagatcagtgaagacagcaatcacggttctgcccctctctccctgtcctcagaccctggaaaagtgaaccacaagactccctccagtgaggagctgggaggagacatcccaggaaaagaatttgatactcctctgtgaatgctcctgccaggccttcagaaattgcatggccactccagcgtcatcggactctctcttattacaaagatcactgcccaggaccatcttcccgagaagcatcccttagcctaaaatccacaccaaagggagagttccagaggaatccatgaagaattcccatgcccaggctccatgtgtcatgtggaaacctccacaggtctgctagtgaagaatgcatgtatgtgagatttttgttttctttccaatagcaaattcaaagcaagcaacttgcaggctccatggaacttttaatgaaggacagtgtcttctttgaagaaaatcaagctcgtgtttttattcgaagctctggtgtaaaatatttcaaagtcatagaaatagtttgagaaatgcatagcattatttaacactattgaacagccgactttgagcattgtttcttctaactgcccctcaactaccattatcttcaagtcaacatgcatattacattttcatcctttgctttgcaagcactggtggcttgcagtttgctaatttatttatcatagagtcatcaatgtatttgttgctgacatggttttattagatactgtagtgatcaaataagttttctatttgaaaaaaaaaatttgatcttgcccagtgaagccatcctaagacttagcaatatggattgtacatttggctgcatgagcaagtcggccgcacacttccagacagtgtgctgtttgaattgactatttgcactcaaagtctgggtattcattggttattggcctgaaatgatcaaataactacaaatgatctgttgaataaaaatagttgagctgatatatgttaagcagatattcaatcagaatgaacaggttccggtggttattttgccgtttgacattttttatggttcatttatttttaatatagagaggaagattgaatatttatctagagaatacaaagacccacatgtaaatgataggtattatctccatgtatatatgtacccacttagtcatgtaagtgcatatacacatacacacacatgagtgtagacatgtgtttattaattgacaatgacccaaatctcttccacaagacttaaaaccaaattcagggacaaatggatagagaagaaaagggtcaaacatcgagattacatggatgttaaattatatggagacgctaagaaataattgatggagccattgatgcaaaccgaagtagatttagaacttatatgaatttgatttatatttttatcagattttaagcttgttttaatggtcaaaaaattaggacagaaataatatggactttattagtatcttccataatttttaagtctgacatttaattcacttaactcattttagatggaatacaaattatatcaaggaaaactgatcaaaagcataattgaaagttctgaaaaaagaaaaataataatatgtagaaaaatgtaacttagagagtaacacatgaacattgaagttaaaacccagaagccagatgctcacagttttattttactttaaaataaacctgtctgactgtagctttggaaatatcttaaaacgcaaaatttatttttaaattttatttaactttaagcaataactagagattacaattaaattttaatcaaaatgaaggcttagttcaaacataaggaaacagtgtttgattaaaaaaaacacatctagtaagacgtaaggggaaaatcacatcctctttggagatgattatattttgatctgaagggtttggggtgttcattagacacttaataaaacttaacttccatgaaaaagaaatcttttgtaaatcattctacttttgcactttgaaagaaaggcgttaatcataaagaagcaagaatggtcaaaatcgaatgctgcatttttataaacaaaattacagactgtctgaaattgaagaagaatgaactaataatagcattcataaccaaacacaatgatgattattgcagaacattgtatcacattttagtccagagatagaataaagttgaataaccttgacttacacaaaactgttttggtagttggatttcattatcttagtgaatttagtcattttacaatatgtttgtatttggccatttactgtaatcacatttttatatctgtacaatgacactttttgcagttgtggggtagtgtgtaacactgtccatcttgcatcattgaaactactacaatgatactatcatttaataatattaatattacttgaaatagactaagataaagaaaaggggtctgtatgatgtgcagttttgtgcctttatgtatttgccttgttctttgtcgaatgtgtgaaattcgtactgtggtttttcctataatagaaagtagagctgtgtattaaattagactgtgtctctctgatacctttacactactgagaatagcatggttttggccatgtaaaccaattttcaaagttctaatgacatagccatgtgtttttggttttttatatttcattttaaaatttgagtatcaccatacattaattaatactcctgtagtagataagctgtcattaagtaattcccaaaaaaagggccatttgcttgcattactttgaatttaatgttgcgcttgtgcactgtgttaatattgtttgtgatggattggacgttgtgactcttgccttttaagaagaaaaaaaagataggacaaagtattt

SEQ ID NO: 27 - DNA, 10331 bp, Homo sapiens
cttgcagcacacaaaataatcagaagaaggtgctagccaaaacactcatttctcttcctcctcaggaagccacaaattcttcgaagccccagccaacctatgaggagctagaggaagaagaacaggaggatcaatttgatttgacagtcgggaagaagaacaggaggatcaatttgatttgacagtcggtataactgatcctgagaagataggggatggtatgaatgcatatgtagcctacaaagttacaacacagggaggtatgaatgcatatgtagcctacaaagttacaacacagacaagcttaccattgttcagaagcaaacagtttgcagtaaaaagaagcttaccattgttcagaagcaaacagtttgcagtaaaaagaagatttagtgactttctgggtctttatgagaagctttccgagaagcactctcagaatggcttcattgtccctcccccccggagaagagcctcatagggatgacaaaagtgaaagttgggaaggaagattcttcttctgcagggacaaaagtgaaagttgggaaggaagattcttcttctgcagaatttcttgaaaaacggagggccgctttagaaaggtaccttcagaggattgtaaatcatcctaccatgttacaggacccaccttcagaggattgtaaatcatcctaccatgttacaggaccctgacgtcagagagttcttggaaaaagaagagctgccacgtgccgtgggtacccagacattgagtggtgctggctgccacgtgccgtgggtacccagacattgagtggtgctggtctcctcaagatgttcaacaaagccacagatgccgtcagcaaaatgaccatcaagatgaatgaatcagacatttggtttgaggagaagctccaggaggtagagtgtgaggagcagcgcttttgaggagaagctccaggaggtagagtgtgaggagcagcgcttacggaaactgcatgctgttgtagaaactctagtcaaccataggaaagagctagcgctgaacacagcccagtttgcaaagagctagtgaactggagatgcgagggcatgctgttccaatcctctcagccatccaagttgaaattctcagcccacctggtccttgatgttctgataaaaaaaagcaacatgattcaaccactgtgggcattgtgctttctacctgggagggggcagggaagccatcccagatacagcatgacctgggatatgggcttctgttgattttgactattacatccaaaatgatgtcatacatgcttgagctacatggtcaaaccagcaatgcgtaaatgcaaggaaaccacctgtctcctaatccttctgcatctctctttagtgaaatttgcttaagagcttgtgaaattttcccagtttgaatcttttccctctttgccttaaacatgctgataattagaaattgaaacagacagaagacccatagcatgaggccagggtaagtaagggttggcaaagtataatctggcccatgggccaaatctgaccctggttcctattgccaaattaggtttttacatttttaaaggaatgaatgagggggaaaagaatctgagcagactatgtgtagccttcaaaatctaaaaaatttattattagccggcttggaggccgaggcaggtgggcacgggcaggagttcaagggcctggccagatggtgaaaccctgtctctgctaaaaaaaaatcagtgtggggtaatcccatctgcttgggaggctgaggcagagaattgcttgaacccgggaggcggaggttacagtgagctgagatggcgccactgcactcctttacacaaagtttgccctactctagaacgtgcaatatacgatgtgcacattattcactcttctctcttagagaggctccaatagtttctttggcctgtttgttatttaacaactgggctttggttctccagggtattggagctgcttccttcaggtaggcctaacccagtgaaactcaggacagctggtgcctctccctcctgctcctgtgaccaggccttctctttacgttaactgtggtgtaagtgactgggttgcagagccctcagttcccagatatgcagtgggcggcattgctaagacatgccatgcagtgtcagctgatttgtcccttttccttgtgagatcagagaggcctctgctgtcttttcctccttccagctagcgctgaacacagcccagtttgcaaagagtctagccatgcttgggagctctgaggacaacacggcattgtcacgggcactctcccagctggctgaggtggaagaaaaaattgagcagctccaccaggaacaggccaacaatgacttcttcctccttgctgagctcctgagtgactacattcgcctcctggccatagtccgcgctgccttcgaccagcgcatgaagacatggcagcgctggcattcctttgctgtccccccgacagtttcccaatctattcacttagctagttatcactgaatcattttgagccccaccaagattcctagatggcatttggagaaaagaccactcttgaatttccgaataggttgcccttttacttcggtcctttcttgaagcaaggttgctgcctctcataacggcttgctgacttccacttgtccttccagtggggcaggaatggctggattgctgggggcagggggagagcttgtcaccagagccttcctgtgctgtgtcaccccacactggcttccagcccccgtcaagcccttttctcgctgtcgcatggcctccctaagacagaggaaagaaccaagaggaagcctggccctagagaagagcaaaggtttcttttctgctcttccctgagcatggagccgggcagggagcagcagtgacaatcctgggggaattcttgcattcgttttggcaaaagcctgtgctgaggaaatattcggggaacttttgcagggcagtgagggagccgctggaacccgggtcagcagcagcttccttccctggaaagctgattgtggtcaaggactggaaacaagtggtagcctagagcaggtgagtgtgtggttgggaaaaagcaatgtggcctggggttttaggtgttgatttaaagattggctaagctaggcctctggtttcatgttatagtaccctgctgctcagtagtctgcatacttgtatctatttgtaattgtaaatgtaatgttggcttcgggacctgagtttcatccactgtattcttagctcctggcacatggcaggcactcagggaaagtatgtagccttatgaactgactgatctgagggaggcgccactggatccctgccatccagccctgggagtagcatgaagcagcatggcactggcttctcccgggccaatgtggctgcagaacctgcccttgctcagtctgtccctggtgcagctgccttcgaccagcgcatgaagacatggcagcgctggcaggatgcccaagccacactgcagaagaagcgggaggccgaggctcggctgctgtgggccaacaagcctgataagctgcagcaggccaaggacgagatcctcgagtgggagtctcgggtgactcaatatgaaagggacttcgagaggagggagtctcgggtgactcaatatgaaagggacttcgagaggatttcaacagtggtccgaaaagaagtgatacggtttgagaaagagaaatccaaggacttcaagaaccacgtgatcaagtaccttgaaaagagaaatccaaggacttcaagaaccacgtgatcaagtaccttgagacactcctttactcacagcagcagctggcaaagtactgggaagccttccttcctgagcccagcgccccgcaagtgcaggccctgcttctgaggggctgggccctcctatagtcctcattgaattttaggcccaggtctgctgccctcattctgaggtgagttagtttttgaggacaaaccccatctccctgtaactgcttacccttcgaggagccaccccatggtaccacctggccctcctgaggctgaggcctcatcggtatgtgaaacagtggccccactgtgaggaactaactgagccaggcaaagctgtggccaccacaggggtgctccctctggggtggagccaagactgtcttcagagactgcccttgcacttggggctcagtaactgctgccttccctgtcgagagggaggtgtgaaggtcatgtggcctgtgcagctgctcggagcctgcctttgtgtggcaggcgcttggggagcgccagggtactgtgcttgattcctcccaggctggcttaggctctgccactagggggggcacttgcttaatgtccccacttctttgcagctggcaaagtactgggaagccttccttcctgaggcaaaggccatctcctaatggaccaaggaccccagagcccacctgtgtgacgctgcctttttatacactgtcctcctccaccttgatggacccctagtgatgcatcctgcctaggctggacttaaccccttcctccctgtccccacgaccaactgtccccagttactctaaccgttatttcatttagcttccatatatattttcttacctaagagaatagtttcctgctttaagcaaaagacctacaataggtggtggaattatgggatggggtggagtattgatataaatatataaatacaaatgtatatttttcaggatgtggtttaggaactgggaataacgttttctgttactcctgatggtgccatgaaaaggttatgtaataaaatattttaaaatcagacatgactcagaatgttggtggtttttgcttaggctggggagcagttgggaatcaggtctggaatactcctaaccaagaagttgcccaggtggtttttctctaccgttcacaagttttgtgctgctgcttccctctggaaatggggtttctttctctccgcctacctcagctacctgttctgagggtctcaatctgtttcgtattcccacttctttagggaaggagttttaaaaacatctcttaaaataagaggagcaaaatctattaaaacctattctcctgcaaaggaggcagagactttctctctctcttttttttttttttttaacaagagcctttctcttttattcttcctgcttccctaagctgctcagggtctctgagtcttgccctctgatggcaagtcttatatataactaaacctatttttgtcacccatcaaaacacatcctcagtagactgtgtgaaggtgtgaaggtctgataatgacttgatgctttatctccatagacatgaaagccatgccctctgcctctagatagggtgatccaagagctcctgaaccttaggaggttcaaagaagctctactgtctgtgcccaggaggtagcctgccagcaagagccctcaggagttgcacacacagccaaagggtgttcacacagatctctgccggtctagccaggggaggccagagtctcgtcagtcaaggatgggcttcccccttagctgtgtccacagctgctcaagctatactggtcagagtgggctttgaagctcctttgtgagctcgagctgctgactgccactatgggagccttgccacctccagcccctccatcccaaagacgctcctgccactggggccccaggtcctgctgtatcagttctctttggtggggggctaaggtttggggcgaggcaacctgagacaagaaaacgcagtaaacattctgattccctgtacacagatgcagcaccaggggaagggccagtggtgcaagtatttctttttaacaggtgaagtttttggaaaaagtcactctccctacccctcagtatccttaccatcaactttggttttatccttccagtctttattatatgcttgcttttacatagttgtaataatatacacataaagtattttgtatcctgctattgttatatatcttcacaaagttgatctgtaaagctgtgtaatttgaagggctgtaccaaatttgattcatcccttgttgttggattcaatgtcctttgtagccattcttttaccttgtgcaggatcatgttacatttgtaaacgtgtgtctctcaatactgcagattcctcactttcttttgtcttttgaagaataggcatttcgaatgttcctcagtttggatttgtgtgatggttccgcatgactaggtgagggttttgcattctgggtgggaatatcacagaagcagtgtgtcgtcagtgcgtcgtaatcagcatataatgtcagttggtcccattaatgttgttaactctgttcaccgcttaggttggtgtctgcctttaaatttacttaatttacaagtaatttgtggggagatactttgagactatataagtattttgttcctcattcaccagttatatagtatcagattagtcttgtcccagttattactgtcatgattgaccaacatgatttcctaactccataatgccttctacatttattggttgacattctacatacaaagaaaaacatcctttctcaccatttatttattcacttgtttatcaatatggatgcagggattcctgttttactcagagttgcaagccattgctttgattttatcttgattctcaaattgtcccagatttatagcgcctcttgtgtgtttgtagcttgctcccatcatgttttgagtactttcttaccttctggcataacaagatgttccaggctcatcttgaatgttccctaccctagccctggaatcagccatttctccagggagtcttggttccttataatggagaatggtatttatttatttttatttgatttgattattattactatttttgagacagagtctcgctcttgtcgcccaggctggattgcaatggcacaatctcagctcactgcaacctccacctaccaggttcaagcaattctcctgcctcagccttccaagtagctgggattacaggtgcctgcccccacacccagctaatttttgcatttttggtagaaatgggggttttaccatgttggggaggctggtctcgaactcctgacctcaggtgatctgcccacctcaacctcccaaagtgctgggattacaggtgtgagccaccatacccagccagagaatgttatttagaaaccaagatctgggtgccaaatgtgctgcttgttactggaatgtcattgcctctaggctctcaccatacagtgaaatatagatagatagatagattgattgatttgtagaaacaagataggttagagatacctgacataaatagatactaaaaatcatgaattcacactgatgctttgagaccagccctgtaggtttattccagtctgcccccttttccatgttgttaactctttcctccagagagacaaagatttcaggttgctttgttaagttccatctaatgatcattctgacgtaagtctgtttttcttatttccttggaatgatgtctcctctggtttcagaacttcctcctctgcttcctgtatcctgaggctggcggggccagttgtctttagggcttgtgcatttttgtaaagagcttgcacgtgtggaaatcaagtaggccagtagtgggttaggggtactgagccagaagcctctacaaggaataacaggagcacaaaggaagaaggtggtattccagctggggacccaggagggaggactttgtggagaacctgatgcttgaactgagtctaaaaggtgtaaaagtgtttgttgcttctgcctccctgtctgtctggcagggtgaggtaggcgcatctagggaaatgtcaagtggcttggtgtagggtaaagtcagtgaggcccatggagaaaaacgagcaggagccacatcacatgggtgtctgataggacctgggaggcgctttccacattaccattgtcgcttcgtgatctggacacaccagaaggcgtgagactggaggcaggaagagcagccaggcttatccctaccctcaggagagctgaaaagggcaggtatggtggggccagagctcaggagagtttcggaaccactgagatcggtccttgatttgatgagaggcttgaggggagagggaggtagctaggatgccccgcaagcttctggcccagacactgggcagacaatgaaaccctttgtaacacatgaggcaataggtttggggcagatgggaggggagcagtggtgggggcagtgagtgctgaaggtgttttaagaagcggctctgggccaggcacagtggcttatgcctgtattcctagcattttgggaggccgaggtgggagaatcacttgagcccaagaatttgagaccagcctggggaatatagtgagaccctgtccctacaaaaataaaaacaactagctgggtgtgtggtggtgcatgcctgtaggcccagctacgcgggaacatcacctgagcccaggaggttgaggttgcagtgagctacagttgcgccactgcactccagcctaggtgacagagcaagatcttgtctcaaaaaaaaaactggggaagggaggccagttgctttaagtaggggagatagagttaaaggaggctttgttttatttaaaggtgggacaaacttaagcatgttaataaaattcagagaagagaaagagaatgactatcagagccatgtttggaagaaaatggggtccagagcacaggaaggggacctgtgttcagagggtgcctcactgctgaggccacaggaaagaatctgtaggtggaggggaggccgaagaggggaagtttcatgcttgataattaaaattttctgagataggaatgtcatatttacctatttaagccaagtttttttagataaaaggtatggaacctgctttccccttggctagttcagcgtttgggctccggagtgctgaagatgaggactggacttcgagctggtgtgatcccagtattcagtgtcagtactcagtgacaaaataaatgagagaaacgggaataagaattgtcgcctacacaaaaataccagcaactgttaactcttcccagaagattttcattctgaatgctcctgtagctaggaaccctaaaaagtctttgaagcaactcaagttttaaaaaaggggaggaactcctggaaatctcaggatggggccaagatgtggctggagagtgtgtggtgatggagggcgtgtcttttgccgagcacactcagggcccacgggaagcccatagacttcaaggacatcaagccccaaggtggtgggattttccccaccagtacttggcagccaggtggggatccctggctccaaacataggaggacacatctgtgctacagtgcgcacatgcctggatgtacactctgtctttggagacactggctaagattctctgctccatgtttggacagggtcgtgcctgatctgagataaatggacaagaacaactgaagcctgtcttctggtgcatgtgtcacctgccgataactgcatcttgtgataaagttgggtgatttacagtctccaccaaatgctaaactctggggtcttacgcctttataactccatgggccccagcaaaggttcaggctcaaaacaggtgtcaaatagataactgttgaatgattgttccccagttgcaggctctgccacctggcgttcatactgtctgtgaaaggacccagctcacctttccctctttatctcccagtccttcccaacagcgccgacacctcatggaaactgattgcaaatgtgctacttctcacttctgtgtggcccgaggaggctgggttaatgctgggcttggtaccttaagcaccctttctcccttccccatcttcattctcagaattacacctgtctgaagcaggcattttccaatgccctagatgggaatataagtgtaaggagatgtgaagcatttgcctgtgtgtcagaacattcactgaggatcctcataggcacttctagaaaccaaatccttgaagatgactaaccagaaatgcccgtcatagcactgtttacagttgcaaaaactgaagccaattgaaatgtccatcaggaggggattaaatgaattatggtacagttacaccgttgaatattttacagccattgaagatgatatatagctatattcattgacaaggaaaactcatattttttagtgaaaaaagcaggttatagaattgcatgatattcacatttatataaaactttatatatgggaaggatgttgattgaattgttaataactatggtcacctctagagatggaagtttgcattaccttaattttt

SEQ ID NO: 28 - DNA, 21 bp, Artificial Sequence
  synthesized shRNA3
cacaggtgattatgatttaaa

SEQ ID NO: 29 - DNA, 19 bp, Artificial Sequence
  synthesized shRNA5
cacattcagtcaacattta

(19) United States
(12) Patent Application Publication
(10) Pub. No.: US 2022/0145395 A1
(43) Pub. Date: May 12, 2022

(54) Transpochimeric Gene Transcripts (TCGTS) As Cancer Biomarkers

(71) Applicant: ECOLE POLYTECHNIQUE FEDERALE DE LAUSANNE (EPFL), Lausanne (CH)

(72) Inventors: Didier TRONO, VUFFLENS-CHATEAU (CH); Laia SIMO RIUDALBAS, Lausanne (CH); Evarist PLANET LETSCHERT, St-Saphorin-Sur-Morges (CH); Julien DUC, Prilly (CH)

(21) Appl. No.: 17/419,990

(22) PCT Filed: Jan. 3, 2020
(86) PCT No.: PCT/EP2020/050073

§ 371 Date: Jun. 30, 2021

(30) Foreign Application Priority Data
Jan. 3, 2019 (EP) 19150151.9

(51) Int. Cl. C12Q 1/6886

(52) U.S. Cl. CPC C12Q 1/6886; C12Q 2600/112; C12Q 2600/158; C12Q 2600/156; C12Q 2600/118

(57) ABSTRACT

The invention relates to methods for detecting or predicting the likelihood of a cancer in a subject as well as to compositions, pharmaceutical compositions and methods of treatment of cancer.

### FIELD OF THE INVENTION

The invention relates to methods for detecting or predicting the likelihood of a cancer in a subject as well as to compositions, pharmaceutical compositions and methods of treatment of cancer.

### REFERENCE TO A SEQUENCE LISTING

This application contains references to amino acid sequences and/or nucleic acid sequences as an ASCII text file. The name of the ASCII text file is “62899388_1.TXT”. It was created on 19 Aug. 2021 and is 128 KB. The aforementioned sequence listing is hereby incorporated by reference in its entirety pursuant to 37 C.F.R. § 1.52(e).

### BACKGROUND OF THE INVENTION

Overall, current cancer drugs are inefficient in close to 75% of patients, and it is increasingly recognized that the future of cancer management lies in personalized approaches, whereby therapies are selected not just based on a tumor's organ of origin but rather on a precise characterization of its molecular make-up and of the patient's genetic background. New methods are needed to discover novel therapeutic targets as well as biomarkers for staging tumors, predicting their sensitivity to particular treatments, monitoring therapeutic responses and detecting relapses as early as possible.

A newly available signature for an in-depth characterization of healthy and tumoral human cells is the expression pattern of transposable elements (TEs). TEs, some 4.5 millions of which can be readily identified in the human genome, may account for up to 80% of our DNA, hence are the repository of a major part of our genetic heritage. TEs have been linked to cancer through different mechanisms, such as insertional activation of oncogenes and disruption of tumor suppressor genes, chromosomal rearrangements, or TE-gene chimeric transcripts that activate oncogenes. Particular TEs have been found overexpressed in cancer but, due to a lack of proper technologies, a comprehensive characterization of their global expression patterns in human cancer is far from complete. The use of improved DNA and RNA sequencing (RNA-seq) methodology combined with in-house optimized bioinformatics pipelines has allowed us to define and start deciphering the TE-derived transcriptome in healthy and cancerous human tissues.

Colorectal cancer (CRC) has been the focus of our studies because its treatment is an unmet clinical need and its step-wise development has been extremely well documented. Furthermore, samples of tumor and healthy tissues are systematically obtained during surgery. CRC is the third most common cancer in men, the second in women and it represents almost a 10% of the annual global cancer incidence. 90% of new cases occur in people above 50 years and the risk of developing colon cancer is 3 times greater in cases with a family history. The preventable risk factors of CRC include consumption of processed meat, consumption of alcoholic beverages, tobacco smoking, and excess of body fat, whereas consumption of dietary fiber and diary products and increased levels of physical activity decrease the risk. CRC screening can prevent the disease through the detection and removal of precancerous lesions and detect cancer at an early stage, when treatment is usually more successful. Current CRC therapy is based on surgery often accompanied by radio- and chemotherapy. Although advances in therapy over the last 20 years have led to improvements in survival, 50% of all CRC patients die from metastatic disease. The invention described in this application aimed to fill this uncovered clinical need.

### SUMMARY OF THE INVENTION

This object has been achieved by providing a method for detecting and/or predicting the likelihood of a cancer in a subject, the method comprising detecting, directly or indirectly, in a biological sample of said subject, the presence or absence of at least one nucleic acid sequence comprising

i) at least one transposable element (TE) a fragment or variant thereof, and
ii) at least one gene sequence, a fragment or variant thereof, wherein the at least one gene sequence is selected from the group listed in Table 1, or a variant or fragment thereof.

A further object of the present invention is to provide a method for classifying a cancer subject, the method comprising

i) detecting, directly or indirectly, in a biological sample of said subject, the presence or absence of at least one nucleic acid sequence comprising at least one transposable element (TE) or a fragment thereof, and at least one gene sequence, a fragment or variant thereof, wherein the at least one gene sequence is selected from the group listed in Table 1 or a variant or fragment thereof and
ii) classifying the cancer subject
as having a favourable prognosis based on the absence of the at least one nucleic acid sequence in the biological sample or
as having a poor prognosis based on the presence of the at least one nucleic acid sequence in the biological.

A further object of the present invention is to provide a method for classifying a cancer subject, the method comprising

1) detecting, directly or indirectly, in a biological sample of said subject, the presence or absence of
at least one nucleic acid sequence correlating with an increase of survival (POSITIVE PROGNOSIS), said nucleic acid sequence comprising at least one transposable element (TE) a fragment or variant thereof, and at least one gene sequence selected from the group comprising DCP2 and YIPF4, a fragment or variant thereof, and
at least one nucleic acid sequence correlating with a decrease of survival (NEGATIVE PROGNOSIS), said nucleic acid sequence comprising at least one transposable element (TE) a fragment or variant thereof, and at least one gene sequence selected from the group comprising POU5F1B, PLCB1, NXPE4, FAAH2, ME3, SNX1, PKP1 and MBD5, a fragment or variant thereof,
   2) scoring the number of nucleic acid sequences correlating with an increase of survival (POSITIVE PROGNOSIS) and the number of nucleic acid sequences correlating with a decrease of survival (NEGATIVE PROGNOSIS),
   wherein the
number of protective events>number of harmful events is indicative of increase in survival (POSITIVE PROGNOSIS).
number of harmful events>number of protective events is indicative of decrease in survival (NEGATIVE PROGNOSIS).

A further object of the present invention is to provide a method for staging a cancer in a subject, the method comprising measuring, directly or indirectly, in a biological sample of said subject, the level of expression or activity of at least one nucleic acid sequence and comparing said level of expression or activity to a control value or range, wherein said at least one nucleic acid sequence comprises

i) at least one transposable element (TE) a fragment or variant thereof, and
ii) at least one gene sequence, a fragment or variant thereof, wherein the at least one gene sequence is selected from the group listed in Table 1, or a variant or fragment thereof.

### FIGURES

FIG. 1. (a) Representation of patients (rows) expressing TcGTs (columns) in the primary tumor (n=10 genes), which are either directly correlated with a decrease or an increase in survival. For every patient, the number of TcGTs correlating with an increase (protective) of survival minus the ones correlated with a decrease (harmful) of survival is computed. This is shown in the bar at the left of the plot. Patients can have either the same number of protective and harmful (0), more harmful tan protective (1, 2, 3, 4 and 5) or more protective than harmful (−1). (b) The survival rates of these groups of patients are represented here.

FIG. 2. LTR66 (a) and POU5F1B (b) expression increases as disease progresses. Expression of the LTR66-POU5F1B TcGT (documented by RACE-rapid amplification of cDNA ends- in CRC cell lines) tightly correlates with the transition from adenoma to carcinoma. Ad: adenoma; I through IV: stages I to IV carcinoma.

FIG. 3. The presence of the 5 TcGTs of POU5F1B in colon primary tumors is linked to a lower survival (HR=−1.69; 95% CI=[−2.56, −1.12]; Pval=0.0095).

FIG. 4. The presence of the 5 TcGTs of POU5F1B in CRC primary tumors is associated with a decrease in relapse-free survival in stage II and III patients (HR=4.40±3.30; 95% CI=[1.01, 19.14]; Pval=0.048). The relapse-signing event here is distant metastasis, not a local recurrence.

FIG. 5. POU5F1B overexpression in HT29 and SW620 CRC cell lines increases their clonogenicity capacity (a) while the downregulation of POU5F1B in LS1034 CRC cell line using two different shRNAs clearly decreases their clonogenicity capacity (b). SW480-POU5F1B cells present an important metastatic power in vivo compared to SW480-GFP cells, suggesting the role of POU5F1B in metastasis (c,d).

FIG. 6. POU5F1B as a stem cell marker. (a) POU5F1B expression anticorrelates with KLF4, ABCG2 and EPAS1 stem cell genes in CRC, 3 genes whose expression also anticorrelates with the mRNA stemness indices defined by Malta et al^{[10]}. (b) POU5F1B expression correlates with the expression of MYC, CD44, LGR5, EZH2 and CTNNB1 stem cell genes in CRC, 5 genes whose expression also correlates with the mRNA stemness indices defined by Malta et al^{[10}]. In the X axis, the expression of each gene of interest in normalized counts; in the Y axis, the expression of POU5F1B in normalized counts.

FIG. 7. POU5F1B as a mesenchymal marker. (a) POU5F1B expression anticorrelates with the expression of the epithelial markers CDS1, MARVELD3, RBM47, MYO5B and MYO5C. (b) POU5F1B expression correlates with the expression of P4HA3 and SNAI1 mesenchymal genes. In the X axis, the expression of each gene of interest in normalized counts; in the Y axis, the expression of POU5F1B in normalized counts.

FIG. 8. POU5F1B transpochimeric transcripts (in black) are more commonly expressed within the MSS CRC patients, which represent the majority of CRC cases (a). The presence of POU5F1B transpochimeric transcripts has a better predictive power (FIG. 3) than MSI or MSS phenotype (b). POU5F1B transpochimeric transcripts are more informative than MSS/MSI to predict survival and they can therefore also be used as an independent predictor (c).

FIG. 9. POU5F1B transpochimeric transcripts (in black) are expressed in all CMS groups.

FIG. 10. POU5F1B expression in metastatic lesions coming from different primary tumor sites. Several metastases originated from diverse tumor types express POU5F1B transpochimeric transcripts and have high expression of the gene (black dots). The metastatic samples with lower expression of POU5F1B do not present POU5F1B transpochimeric transcripts (grey diamonds). None of the primary tumors (empty circles) present POU5F1B transpochimeric transcripts.

FIG. 11. POU5F1B expression in the normal colon samples coming from the 300 CRC patients cohort, in the CRC primary tumors not expressing POU5F1B transpochimeric transcripts, in the CRC primary tumors expressing them, and in all the rest of human normal tissues. The expression of POU5F1B is clearly higher in the CRC primary tumors expressing POU5F1B transpochimeric transcripts compared to the rest of samples.

FIG. 12. Schematic representation of the TcGTs a) DCP2, b) FAAH2, c) MBD5, d) ME3, e) NXPE4, f) PKP1, g) PLCB1, h) POU5F1B, i) SNX1, and j) YIPF4.

### DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise/comprising” is generally used in the sense of include/including, that is to say permitting the presence of one or more features or components. The terms “comprise” and “comprising” also encompass the more restricted ones “consist” and “consisting”, respectively.

As used herein, the singular form “a”, “an” and “the” include plural references unless the context clearly dictates otherwise.

As used herein, “at least one” means “one or more”, “two or more”, “three or more”, etc. For example, at least one nucleic acid means “one or more nucleic acid”, “two or more nucleic acids”, “three or more nucleic acids”, etc.

As used herein the terms “subject”, or “patient” are well-recognized in the art, and, are used interchangeably herein to refer to a mammal, including dog, cat, rat, mouse, monkey, cow, horse, goat, sheep, pig, camel, and, most preferably, a human. In some aspects, the subject is a subject in need of treatment or a subject suffering from cancer or a subject that might be at risk of suffering from cancer. However, in other aspects, the subject can be a normal subject. The term does not denote a particular age or sex. Thus, adult and newborn subjects, whether male or female, are intended to be covered.

According to the present invention, the cancer is selected from the non-limiting group comprising carcinoma, sarcoma, melanoma, lymphoma, and leukemia. Preferably, the cancer is selected from the group comprising Acute Lymphoblastic Leukemia (ALL), Acute Myeloid Leukemia (AML), Adrenocortical Carcinoma, Adult, Childhood Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Astrocytomas, Childhood (Brain Cancer), Atypical Teratoid/Rhabdoid Tumor, Childhood, Central Nervous System (Brain Cancer), Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Childhood Breast Cancer, Childhood Bronchial Tumors, Burkitt Lymphoma, Carcinoid Tumor (Gastrointestinal), Childhood Carcinoid Tumors, Carcinoma of Unknown Primary, Cardiac (Heart) Tumors, Central Nervous System, Childhood Atypical Teratoid/Rhabdoid Tumor, Childhood Embryonal Tumors, Childhood Germ Cell Tumor, Primary CNS Lymphoma, Cervical Cancer, Childhood Cervical Cancer, Cholangiocarcinoma, Chordoma, Childhood, Chronic Lymphocytic Leukemia (CLL), Chronic Myelogenous Leukemia (CML), Chronic Myeloproliferative Neoplasms, Colorectal Cancer, Childhood Colorectal Cancer, Childhood Craniopharyngioma, Cutaneous T-Cell Lymphoma, Ductal Carcinoma In Situ (DCIS), Embryonal Tumors, Endometrial Cancer (Uterine Cancer), Childhood Ependymoma, Esophageal Cancer, Childhood Esophageal Cancer, Esthesioneuroblastoma, Ewing Sarcoma (Bone Cancer), Childhood Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone (Malignant, and Osteosarcoma), Gallbladder Cancer, Gastric, (Stomach) Cancer, Childhood Gastric (Stomach) Cancer, Gastrointestinal Carcinoid Tumor, Gastrointestinal Stromal Tumors (GIST) (Soft Tissue Sarcoma), Childhood Gastrointestinal Stromal Tumors, Germ Cell Tumors, Childhood Central Nervous System Germ Cell Tumors (Brain Cancer), Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Childhood Head and Neck Cancers, Childhood Heart Tumors, Hepatocellular (Liver) Cancer, Langerhans Cell Histiocytosis, Hodgkin Lymphoma, Hypopharyngeal Cancer (Head and Neck Cancer), Intraocular Melanoma, Childhood Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Childhood Laryngeal Cancer, Papillomatosis, Leukemia, Lip and Oral Cavity Cancer (Head and Neck Cancer), Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Childhood Melanoma, Intraocular (Eye) Melanoma, Childhood Intraocular Melanoma, Merkel Cell Carcinoma (Skin Cancer), Malignant Mesothelioma, Childhood Mesothelioma, Metastatic Squamous Neck Cancer with Occult Primary (Head and Neck Cancer), Midline Tract Carcinoma Involving NUT Gene, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplasia Syndromes, Myelodysplastic/Myeloproliferative Neoplasms, Myelogenous Leukemia, Chronic (CML), Myeloid Leukemia, Acute (AML), Chronic Myeloproliferative Neoplasms, Nasal Cavity and Paranasal Sinus Cancer (Head and Neck Cancer), Nasopharyngeal Cancer (Head and Neck Cancer), Childhood Nasopharyngeal Cancer, Neuroblastoma, Non-Hodgkin Lymphoma, Non-Small Cell Lung Cancer, Oral Cancer, Lip and Oral Cavity Cancer and Oropharyngeal Cancer (Head and Neck Cancer), Childhood Oral Cavity Cancer, Osteosarcoma and Malignant Fibrous Histiocytoma of Bone, Ovarian Cancer, Childhood Ovarian Cancer, Pancreatic Cancer, Childhood Pancreatic Cancer, Pancreatic Neuroendocrine Tumors (Islet Cell Tumors), Papillomatosis, Paraganglioma, Childhood Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Childhood Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Childhood Rhabdomyosarcoma (Soft Tissue Sarcoma), Salivary Gland Cancer (Head and Neck Cancer), Childhood Salivary Gland Tumors, Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Uterine Sarcoma, Sezary Syndrome (Lymphoma), Skin Cancer, Childhood Skin Cancer, Small Cell Lung Cancer, Small Intestine Cancer, Soft Tissue Sarcoma, Squamous Cell Carcinoma of the Skin, Squamous Neck Cancer with Occult Primary, Metastatic (Head and Neck Cancer), Stomach (Gastric) Cancer, Childhood Stomach (Gastric) Cancer, T-Cell Lymphoma, Cutaneous, Testicular Cancer, Childhood Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Childhood Thyroid Tumors, Transitional Cell Cancer of the Renal Pelvis and Ureter (Kidney (Renal Cell) Cancer), Carcinoma of Unknown Primary, Childhood Cancer of Unknown Primary, Unusual Cancers of Childhood, Ureter and Renal Pelvis, Transitional Cell Cancer (Kidney (Renal Cell) Cancer, Urethral Cancer, Endometrial Uterine Cancer, Uterine Sarcoma, Vaginal Cancer, Childhood Vaginal Cancer, Vascular Tumors (Soft Tissue Sarcoma), Vulvar Cancer, and Wilms Tumor or a combination of one of more of these cancers

More preferably, the cancer is a solid tumor cancer. Most preferably, the solid tumor cancer is selected from the group comprising Colorectal Cancer, Gastric Cancer, Prostate Cancer, Liver Cancer, Testis Cancer, Ovary Cancer and Breast Cancer, or a combination of one of more of these cancers. Even more preferably, the cancer is Colorectal Cancer.

The terms “nucleic acid”, “polynucleotide,” and “oligonucleotide” are used interchangeably and refer to any kind of deoxyribonucleotide (e.g. DNA, cDNA, . . . ) or ribonucleotide (e.g. RNA, mRNA, . . . ) polymer or a combination of deoxyribonucleotide and ribonucleotide (e.g. DNA/RNA) polymer, in linear or circular conformation, and in either single- or double-stranded form. These terms are not to be construed as limiting with respect to the length of a polymer and can encompass known analogues of natural nucleotides, as well as nucleotides that are modified in the base, sugar and/or phosphate moieties (e.g. phosphorothioate backbones). In general, an analogue of a particular nucleotide has the same base-pairing specificity; i.e., an analogue of A will base-pair with T.

In one aspect the acid nucleic of the invention is an acid nucleic comprising i) at least one transposable element (TE), a fragment or variant thereof, and a ii) at least one gene sequence, a fragment or variant thereof. The acid nucleic of the invention is preferably selected from the group comprising an RNA transcript, a cDNA of said RNA transcript or a DNA sequence, preferably an amplified DNA sequence. In some aspects the acid nucleic of the invention comprising i) at least one transposable element (TE), a fragment or variant thereof, and ii) at least one gene sequence, a fragment or variant thereof, is an RNA transcript called “transpochimera” or “Transpochimeric gene transcripts (TcGTs)”.

As used herein, a “biological sample” refers to a sample of tissue or fluid isolated from a subject, including but not limited to, for example, urine, blood, plasma, serum, fecal matter, bone marrow, bile, spinal fluid, lymph fluid, samples of the skin, external secretions of the skin, respiratory, intestinal, and genitourinary tracts, tears, saliva, milk, blood cells, organs, biopsies, and also samples containing cells or tissues derived from the subject and grown in culture, and in vitro cell culture constituents, including but not limited to, conditioned media resulting from the growth of cells and tissues in culture, recombinant cells, stem cells, and cell components.

Surprisingly, the inventors of the present invention have shown that transposable element (TE) transcripts fused to complete or partial gene transcripts can serve as cancer biomarkers and source of therapeutic targets for cancer.

In one aspect, the present invention thus provides a method for detecting and/or predicting the likelihood of a cancer in a subject, the method comprising detecting, directly or indirectly, in a biological sample of said subject, the presence or absence of at least one nucleic acid sequence comprising i) at least one transposable element (TE) a fragment or variant thereof, and ii) at least one gene sequence, a fragment or variant thereof. Preferably, the at least one gene sequence, fragment or variant thereof, will be selected from the group comprising POU5F1B, PLCB1, NXPE4, FAAH2, ME3, SNX1, PKP1, MBD5, DCP2 and YIPF4, or a combination of one more thereof. More preferably, the at least one gene sequence is a POU5F1B sequence, a fragment or variant thereof.

Non-limiting examples of combinations of one or more gene sequence, fragment or variant thereof, comprise two or more, three or more, four or more, five or more, six or more, seven or more, eight or more, nine or more, etc. . . . . gene sequences, fragments) or variant(s) thereof. Combinations of gene sequences, fragments and/or variants are also comprised within the present invention. Preferably, the combination comprises at least one or more POU5F1B sequence, fragment or variant thereof. Even more preferably, the combination comprises at least one or more POU5F1B sequence, fragment or variant thereof, and one or more gene sequence (e.g. 1, 2, 3, 4, 5, 6, 7, 8, or 9) selected from the group comprising PLCB1, NXPE4, FAAH2, ME3, SNX1, PKP1, MBD5, DCP2 and YIPF4, or a combination of one more thereof.

Usually, the at least one nucleic acid is selected from the group listed in Table 1, or a variant or fragment thereof. In one aspect, the at least one nucleic acid is selected from the group comprising SEQ ID No 1, 2, 3, 4, 5, 6, 7, 8, 9, 10, 11, 12, 13, 14, 15, 16, 17, 18, 19, 20, 21, 22, 23, 24, 25, 26, and 27, a fragment or variant thereof or a combination of one or more thereof. The presence of at least one nucleic acid sequence predictor of poor survival (harmful predictor, identified by −), and/or the absence of at least one predictor of good survival (protective predictor, identified by +) being indicative of the presence of a cancer and/or of a poor prognosis of said cancer.

It is understood that the presence of the at least one nucleic acid sequence comprising i) at least one transposable element (TE) a fragment or variant thereof, and ii) at least one gene sequence, a fragment or variant thereof, in a biological sample can be determined by any suitable method known in the art. Detection of said at least one nucleic acid sequence can be direct or indirect.

For example, the presence of the at least one nucleic acid sequence comprising i) at least one transposable element (TE) a fragment or variant thereof, and ii) at least one gene sequence, a fragment or variant thereof, can be directly detected using, e.g. Northern blot analysis or RNA FISH technique.

Alternatively, the presence of said at least one nucleic acid sequence can be determined indirectly by detecting the presence of cDNAs, amplified RNAs or DNAs, or by measuring activities of RNAs, or other molecules that are indicative of the expression level of said at least one nucleic acid sequence such as, e.g. peptides or proteins. Preferably, the presence or absence of said at least one nucleic acid sequence is determined indirectly by detecting the presence of the corresponding cDNAs.

The one or more acid nucleic of the invention can be detected and, alternatively, quantitated by a variety of methods including, but not limited to, microarray analysis, polymerase chain reaction (PCR), reverse transcriptase polymerase chain reaction (RT-PCR), Northern blot, serial analysis of gene expression (SAGE), immunoassay, mass spectrometry, and any sequencing-based methods known in the art (such as e.g. RNA-seq).

In a particular aspect, microarrays are used to measure the presence and/or levels of the at least one or more acid nucleic of the invention. An advantage of microarray analysis is that the expression of each of the one or more acid nucleic of the invention can be measured simultaneously, and microarrays can be specifically designed to provide a diagnostic expression profile for a particular disease or condition (e.g., a type of cancer, stage of cancer, . . . ).

Microarrays are prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

Probes that selectively hybridize the at least one or more acid nucleic of the invention and used in the methods of the invention are preferably immobilized to a solid support which may be either porous or non-porous. For example, the probes may be polynucleotide sequences which are attached to a nitrocellulose or nylon membrane or filter covalently at either the 3′ or the 5′ end of the polynucleotide. Such hybridization probes are well known in the art (see, e.g., Sambrook, et al., Molecular Cloning: A Laboratory Manual (3rd Edition, 2001). Alternatively, the solid support or surface may be a glass or plastic surface. In one embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel. In one embodiment, the microarray comprises a support or surface with an ordered array of binding (e.g., hybridization) sites or “probes” each representing one of the biomarkers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). Each probe is preferably covalently attached to the solid support at a single site. Microarrays can be made in a number of ways, of which several are described below. However they are produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. Microarrays are generally small, e.g., between 1 cm2 and 25 cm2; however, larger arrays may also be used, e.g., in screening arrays. Preferably, a given binding site or unique set of binding sites in the microarray will specifically recognize and bind (e.g., hybridize) to the product of a single gene in a cell (e.g., to a specific mRNA or to a specific cDNA derived therefrom). However, in general, other related or similar sequences will cross hybridize to a given binding site.

As noted above, the “probe” to which a particular polynucleotide molecule specifically hybridizes contains a complementary polynucleotide sequence. The probes of the microarray typically consist of nucleotide sequences of no more than 1,000 nucleotides. In some embodiments, the probes of the array consist of nucleotide sequences of 10 to 1,000 nucleotides. In one aspect, the nucleotide sequences of the probes are in the range of 10-200 nucleotides in length and are genomic sequences of one species of organism, such that a plurality of different probes is present, with sequences complementary and thus capable of hybridizing to the genome of such a species of organism, sequentially tiled across all or a portion of the genome. In other embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, or are 60 nucleotides in length. The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone (e.g., phosphorothioates).

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., PCR Protocols: A Guide To Methods And Applications, Academic Press Inc., San Diego, Calif. (1990); herein incorporated by reference in its entirety. It will be apparent to one skilled in the art that controlled robotic systems are useful for isolating and amplifying nucleic acids.

An alternative, preferred means for generating polynucleotide probes is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., U.S. Pat. No. 5,539,083).

Probes are preferably selected using an algorithm that takes into account binding energies, base composition, sequence complexity, cross-hybridization binding energies, and secondary structure. See e.g. International Patent Publication WO 01/05935.

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

The probes are attached to a solid support or surface, which may be made, e.g., from glass, plastic (e.g., polypropylene, nylon), polyacrylamide, nitrocellulose, gel, or other porous or nonporous material. One method for attaching nucleic acids to a surface is by printing on glass plates, as known in the art. This method is especially useful for preparing microarrays of cDNA. A second method for making microarrays produces high-density oligonucleotide arrays. Techniques are known for producing arrays containing thousands of oligonucleotides complementary to defined sequences, at defined locations on a surface using photo-lithographic techniques for synthesis in situ (see, U.S. Pat. Nos. 5,578,832; 5,556,752; and 5,510,270; herein incorporated by reference in their entireties) or other methods for rapid synthesis and deposition of defined oligonucleotides. When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking, may also be used. In principle, any type of array known in the art, for example, dot blots on a nylon hybridization membrane could be used. However, as will be recognized by those skilled in the art, very small arrays will frequently be preferred because hybridization volumes will be smaller.

Microarrays can also be manufactured by means of an ink jet printing device for oligonucleotide synthesis, e.g., using the methods and systems described by Blanchard in U.S. Pat. No. 6,028,189. Specifically, the oligonucleotide probes in such microarrays are synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Microarrays manufactured by this ink jet method are typically of high density, preferably having a density of at least about 2,500 different probes per 1 cm2. The polynucleotide probes are attached to the support covalently at either the 3′ or the 5′ end of the polynucleotide.

The at least one nucleic acid sequence of the invention comprising i) at least one transposable element (TE) a fragment or variant thereof, and a ii) at least one gene sequence, a fragment or variant thereof which may be measured by microarray analysis can be expressed RNAs or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one aspect, the target polynucleotide molecules comprise RNA, including, but by no means limited to, total cellular RNA, poly(A)+ messenger RNA (mRNA) or a fraction thereof, cytoplasmic mRNA, or RNA transcribed from cDNA (i.e., cRNA; see, e.g., U.S. Pat. Nos. 5,545,522, 5,891,636, or 5,716,785). Methods for preparing total and poly(A)+ RNA are well known in the art, and are described generally, e.g., in Sambrook, et al., Molecular Cloning: A Laboratory Manual (3rd Edition, 2001). RNA can be extracted from a cell of interest using guanidinium thiocyanate lysis followed by CsCl centrifugation, a silica gel-based column (e.g., RNeasy (Qiagen, Valencia, Calif.) or StrataPrep (Stratagene, La Jolla, Calif.)), or using phenol and chloroform, as known in the art. Poly(A)+ RNA can be selected, e.g., by selection with oligo-dT cellulose or, alternatively, by oligo-dT primed reverse transcription of total cellular RNA. RNA can be fragmented by methods known in the art, e.g., by incubation with ZnCl2, to generate fragments of RNA.

Preferably, the at least one acid nucleic sequence is selected from the group comprising SEQ ID No 1, 2, 3, 4, 5, 6, 7, 8, 9, 10, 11, 12, 13, 14, 15, 16, 17, 18, 19, 20, 21, 22, 23, 24, 25, 26, and 27, a fragment or variant thereof or a combination of one or more thereof.

RNA-Seq is a High-throughput sequencing method recently developed for transcriptome profiling technology that utilizes next-generation sequencing platforms (Metzker, 2010; Mardis, 2008). RNA-Seq transcripts are reverse-transcribed into cDNA, and adapters are ligated to each end of the cDNA. Sequencing can be done either unidirectional (single-end sequencing) or bidirectional (paired-end sequencing) and then aligned to a reference genome database or assembled to obtain de novo transcripts, proving a genome-wide expression profile (Wang et al., Nat. Rev. Genet. (2009) 10:57-63, 2009 and Marguerat and Bahler in Cell. Mol. Life. Sci. (2010) 67:569-579).

As described herein, the nucleic acid sequences of the invention can be detectably labeled at one or more nucleotides. Any method known in the art may be used to label the target the nucleic acid sequences of the invention. Preferably, this labeling incorporates the label uniformly along the length of the RNA, and more preferably, the labeling is carried out at a high degree of efficiency. For example, nucleic acid sequences can be labeled by oligo-dT primed reverse transcription. Random primers (e.g., 9-mers) can be used in reverse transcription to uniformly incorporate labeled nucleotides over the full length of the nucleic acid sequences. Alternatively, random primers may be used in conjunction with PCR methods or T7 promoter-based in vitro transcription methods in order to amplify the nucleic acid sequences of the invention.

The detectable label may be a luminescent label. For example, fluorescent labels, bioluminescent labels, chemiluminescent labels, and colorimetric labels may be used in the practice of the invention. Fluorescent labels that can be used include, but are not limited to, fluorescein, a phosphor, a rhodamine, or a polymethine dye derivative. Additionally, commercially available fluorescent labels including, but not limited to, fluorescent phosphoramidites such as FluorePrime (Amersham Pharmacia, Piscataway, N.J.), Fluoredite (Miilipore, Bedford, Mass.), FAM (ABI, Foster City, Calif.), and Cy3 or Cy5 (Amersham Pharmacia, Piscataway, N.J.) can be used. Alternatively, the detectable label can be a radiolabeled nucleotide.

In one aspect, nucleic acid sequences from a patient sample are labeled differentially from the corresponding polynucleotide molecules of a reference sample. The reference can comprise RNAs from a normal biological sample (i.e., control sample, e.g., biopsy from a subject not having a cancer) or from a reference biological sample, (e.g., sample from a subject having a cancer).

Nucleic acid hybridization and wash conditions are chosen so that the target nucleic acid sequences specifically bind or specifically hybridize to the complementary polynucleotide sequences of the array, preferably to a specific array site, wherein its complementary DNA is located. Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self-complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook, et al., Molecular Cloning: A Laboratory Manual (3rd Edition, 2001). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS). Particularly preferred hybridization conditions include hybridization at a temperature at or near the mean melting temperature of the probes (e.g., within 51° C., more preferably within 21° C.) in 1 M NaCl, 50 mM MES buffer (pH 6.5), 0.5% sodium sarcosine and 30% formamide.

When fluorescently labeled gene products are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously. Arrays can be scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are known in the art. Alternatively, a fiber-optic bundle, may be used to monitor mRNA abundance levels at a large number of sites simultaneously.

In one aspect, the invention includes a microarray comprising a plurality of probes that selectively hybridize to one or more nucleic acid of the invention. Preferably, one or more nucleic acid of the invention are selected from the group listed in Table 1, fragments thereof, isoforms thereof and variants sharing at least 80% nucleotide sequence identity thereto, most preferably from the group comprising SEQ ID No 1, 2, 3, 4, 5, 6, 7, 8, 9, 10, 11, 12, 13, 14, 15, 16, 17, 18, 19, 20, 21, 22, 23, 24, 25, 26, and 27.

Serial Analysis Gene Expression (SAGE), can also be used to determine the presence and/or abundance of an RNA (e.g., transpochimera) in a cell sample.

Quantitative reverse transcriptase PCR (qRT-PCR) can also be used to determine the presence and/or expression profiles of the nucleic acid sequences of the invention (see, e.g., U.S. Patent Application Publication No. 2005/0048542A1; herein incorporated by reference in its entirety). The first step is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN RT-PCR can be performed using commercially available equipment, such as, for example, ABI PRISM 7700 sequence detection system. (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700 sequence detection system. The system consists of a thermocycler, laser, charge-coupled device (CCD), camera and computer. The system includes software for running the instrument and for analyzing the data. 5′-Nuclease assay data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and beta-actin.

Another variation of the RT-PCR technique is the real time quantitative PCR, which measures PCR product accumulation through a dual-labeled fluorigenic probe (i.e., TAQMAN probe). Real time PCR is compatible both with quantitative competitive PCR, where internal competitor for each target sequence is used for normalization, and with quantitative comparative PCR using a normalization gene contained within the sample, or a housekeeping gene for RT-PCR.

Mass spectrometry, and particularly SELDI mass spectrometry, is a particularly useful method for detection of one or more nucleic acid (e.g. biomarkers) of this invention. Laser desorption time-of-flight mass spectrometer can be used in embodiments of the invention. In laser desorption mass spectrometry, a substrate or a probe comprising biomarkers is introduced into an inlet system. The biomarkers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry (MALDI-MS) can also be used for detecting the one or more nucleic acid sequence of this invention. MALDI-MS is a method of mass spectrometry that involves the use of an energy absorbing molecule, frequently called a matrix, for desorbing proteins intact from a probe surface. MALDI is described, for example, in U.S. Pat. Nos. 5,118,937 and 5,045,694. In MALDI-MS, the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry.

Surface-enhanced laser desorption/ionization mass spectrometry, or SELDI-MS represents an improvement over MALDI for the fractionation and detection of biomolecules, such as the nucleic acid sequences of the invention, in complex mixtures. SELDI is a method of mass spectrometry in which biomolecules, such as the nucleic acid sequences of the invention, are captured on the surface of a biochip using capture reagents that are bound there. Typically, non-bound molecules are washed from the probe surface before interrogation. SELDI is described, for example, in: U.S. Pat. No. 5,719,060 and in U.S. Pat. No. 6,225,047.

The nucleic acid sequences of the invention on the substrate surface can be desorbed and ionized using gas phase ion spectrometry. Any suitable gas phase ion spectrometer can be used as long as it allows biomarkers on the substrate to be resolved. Preferably, gas phase ion spectrometers allow quantitation of biomarkers. In one embodiment, a gas phase ion spectrometer is a mass spectrometer. In a typical mass spectrometer, a substrate or a probe comprising biomarkers on its surface is introduced into an inlet system of the mass spectrometer. The biomarkers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of the nucleic acid sequences of the invention or other substances will typically involve detection of signal intensity. This, in turn, can reflect the quantity and character of the nucleic acid sequences of the invention bound to the substrate. Any of the components of a mass spectrometer (e.g., a desorption source, a mass analyzer, a detector, etc.) can be combined with other suitable components described herein or others known in the art in embodiments of the invention.

The nucleic acid sequences of the invention can also be detected with assays based on the use of antibodies that specifically recognize the nucleic acid sequences of the invention or oligonucleotide fragments of said nucleic acid sequences of the invention. Such assays include, but are not limited to, immunohistochemistry (IHC), enzyme-linked immunosorbent assay (ELISA), radioimmunoassays (RIA), “sandwich” immunoassays, fluorescent immunoassays, immunoprecipitation assays, the procedures of which are well known in the art.

The nucleic acid sequences of the invention can also be detected with any sequencing based technologies know in the art.

As indicated above, the method of the invention for detecting and/or predicting the likelihood of a cancer in a subject, also comprises detecting, directly or indirectly, in a biological sample of said subject, the presence or absence of a fragment or variant of at least one nucleic acid sequence of the invention that comprises i) at least one transposable element (TE), and a ii) at least one gene sequence. Preferably, the at least one additional nucleic acid is selected from the group listed in Table 1.

As used herein, the term “variant” refers to biologically active derivatives of a nucleic acid sequence. In general, the term “variant” refers to molecules having a native sequence and structure with one or more additions, substitutions (generally conservative in nature) and/or deletions (e.g. splice variants), relative to the native molecule, so long as the modifications do not destroy biological activity and which are “substantially homologous” to the reference molecule. In general, the sequences of such variants are functionally, i.e. biologically, active variants and will have a high degree of sequence homology to the reference sequence, e.g., sequence homology of more than 50%, generally more than 60%-70%, even more particularly 80%-85% or more, such as at least 90% or 95% or more, when the two sequences are aligned.

Alternatively, the term “variant” also refers to post-transcriptionally modified nucleic acid sequence of the invention, i.e methylation, phosphorylation, etc.

As used herein, a “fragment” of one or more nucleic acid sequence or polypeptide sequence of the invention refers to a sequence containing less nucleotides or amino acids in length than the respective sequences of the invention while retaining the biological activity described herein. Preferably, this fragment contains, at least 10%, 20%, 30%, 40%, 50%, 60%, 70%, 80%, or 90% of the entire length of the reference nucleic acid sequence or polypeptide sequence.

As used herein, “Homology” refers to the percent identity between two polynucleotide or two polypeptide sequences. Two nucleic acid, or two polypeptide sequences are “substantially homologous” to each other when the sequences exhibit at least about 50% sequence identity, preferably at least about 75% sequence identity, more preferably at least about 80%-85% sequence identity, more preferably at least about 90% sequence identity, and most preferably at least about 95%-98% sequence identity over a defined length of the molecules. As used herein, substantially homologous also refers to sequences showing complete identity to the specified sequence.

In general, “identity” refers herein to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Sequence identity is typically measured using sequence analysis software (for example, Sequence Analysis Software Package of the Genetics Computer Group, University of Wisconsin Biotechnology Center, 1710 University Avenue, Madison, Wis.53705, BLAST, BESTFIT, GAP, or PILEUP/PRETTYBOX programs). Such software matches identical or similar sequences by assigning degrees of homology to various substitutions, deletions, and/or other modifications.

Alternatively, homology can be determined by readily available computer programs or by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art.

The at least one TE of the invention is located either upstream and/or downstream, i.e. 5′ and/or 3′ regions, preferably upstream, of ii) at least one gene sequence, a fragment or variant thereof and can be, for example, any non-long terminal repeat (LTR) or any LTR of an endogenous retrovirus.

Preferably, the at least one TE is selected from the non-limiting group comprising AluJr4, HAL1, L1PA16, AluSx, L1PA8, L1PA5, L1PA8, L1PA7, AluJr, L1PA7, MIR, L2b, L2c, MIRb, LTR16A, THE1D, MLT2B4, ERVL-B4-int, LTR12, MIR3, L1PA3, L1PA2, L2b, LTR33, LTR66, L2d2, AluSg7 and AluSx, a fragment or variant thereof, or a combination of one or more thereof. Examples of combinations of one or more of the TE can comprise, e.g. one or more of the same TE (LTR66 & LTR66, etc.) or one or more of different TEs (LTR16A & MIR, AluJr4 & HAL1, AluSx & L1PA8, MLT2B4, ERVL-B4-int, etc.).

In one aspect, the at least one TE is a LTR selected from the non-limiting group comprising LTR66, MIR, MIRb, L2b, L2c, and LTR33, a fragment or variant thereof, or a combination of one or more thereof.

The at least one gene sequence, also named associated gene sequence, a fragment or variant thereof of the invention can be any gene sequence.

Preferably, said at least one gene sequence is selected from the group comprising POU5F1B, PLCB1, NXPE4, FAAH2, ME3, SNX1, PKP1, MBD5, DCP2, YIPF4, or a combination of one more thereof.

More preferably, the gene sequence is a POU5FB1 sequence, a fragment or a variant thereof.

The present invention also envisions methods for classifying a cancer subject as having a favourable prognosis or as having a poor prognosis based on the presence or absence of the at least one nucleic acid sequence of the invention.

As used herein, the term “favourable prognosis” or “good prognosis” refers to a patient or subject afflicted with cancer receiving, or not, a treatment that is likely to not present metastasis, and/or that is likely to not present cancer relapse, and/or that is likely to present a high overall survival (OS), event-free survival (EFS), and/or metastasis-free survival (MFS).

As used herein, the term “poor prognosis” or “bad prognosis” refers to a patient or subject afflicted with cancer receiving or not a treatment that is likely to present metastasis, and/or that is likely to present cancer relapse, and/or that is likely to present short overall survival (OS), progression free survival (PFS) and/or metastasis.

In one aspect, the present invention relates to a method for classifying a cancer subject, the method comprising i) detecting, directly or indirectly, in a biological sample of said subject, the presence or absence of at least one nucleic acid sequence comprising at least one transposable element (TE), a fragment or variant thereof, and at least one gene sequence, a fragment or variant thereof, and ii) classifying the cancer subject
as having a favourable prognosis based on the absence of the at least one nucleic acid sequence in the biological sample or
as having a poor prognosis based on the presence of the at least one nucleic acid sequence in the biological.

Preferably, the at least one nucleic acid is selected from the group listed in Table 1, a variant, a fragment or a combination thereof.

Preferably, the at least one nucleic acid sequence is selected from the group comprising:
at least one transposable element (TE) selected from the group comprising AluJr4, HAL1, AluSg7 and AluSx, a variant, a fragment or a combination thereof and
at least one gene sequence selected from the group comprising DCP2 and YIPF4, a fragment, a variant or a combination thereof.

Preferably, the at least one nucleic acid sequence is selected from the group comprising:
at least one transposable element (TE) selected from the group comprising L1PA16,

AluSx, L1PA8, L1PA5, L1PA8, L1PA7, AluJr, L1PA7, MIR, L2b, MIRb, LTR16A, THE1D, MLT2B4, ERVL-B4-int, LTR12, MIR3, L1PA3, L1PA2, L2, LTR33, LTR66 and L2d2, a variant, a fragment or a combination thereof, and
at least one gene sequence selected from the group POU5F1B, PLCB1, NXPE4, FAAH2, ME3, SNX1, PKP1 and MBD5, a fragment, a variant or a combination thereof.

Preferably, the gene sequence is a POU5F1B sequence, a fragment or a variant thereof and the TE is selected from the group comprising LTR66, LTR33, MIR, L2a, L2b, L2c and MIRb, a variant, a fragment or a combination thereof.

Usually, the cancer is selected from the group described above. Preferably, the cancer is a solid tumor cancer. More preferably, the solid tumor cancer is selected from the group comprising Colorectal Cancer, Gastric Cancer, Prostate Cancer, Liver Cancer, Testis Cancer, Ovary Cancer and Breast Cancer, or a combination of one of more of these cancers. Even more preferably, the cancer is Colorectal Cancer.

In another aspect, the present invention further envisions a method for classifying a cancer subject, the method comprising

1) detecting, directly or indirectly, in a biological sample of said subject, the presence or absence of
at least one nucleic acid sequence correlating with an increase of survival (POSITIVE PROGNOSIS), said nucleic acid sequence comprising at least one transposable element (TE), a fragment or variant thereof, and at least one gene sequence selected from the group DCP2 and YIPF4, a fragment or variant thereof, and
at least one nucleic acid sequence correlating with a decrease of survival (NEGATIVE PROGNOSIS), said nucleic acid sequence comprising at least one transposable element (TE), a fragment or variant thereof, and at least one gene sequence selected from the group comprising POU5F1B, PLCB1, NXPE4, FAAH2, ME3, SNX1, PKP1 and MBD5, a fragment or variant thereof,
   2) scoring the number of nucleic acid sequences correlating with an increase of survival (POSITIVE PROGNOSIS) and the number of nucleic acid sequences correlating with a decrease of survival (NEGATIVE PROGNOSIS), wherein the
number of protective events>number of harmful events is indicative of increase in survival (POSITIVE PROGNOSIS), and
number of harmful events>number of protective events is indicative of decrease in survival (NEGATIVE PROGNOSIS).

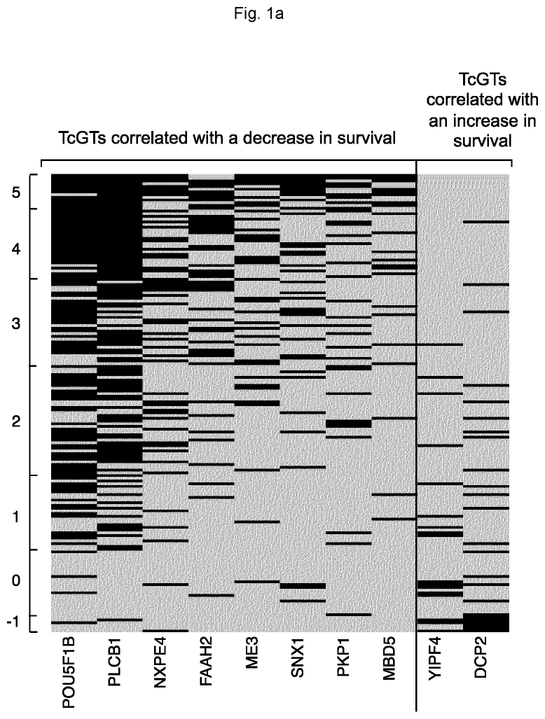

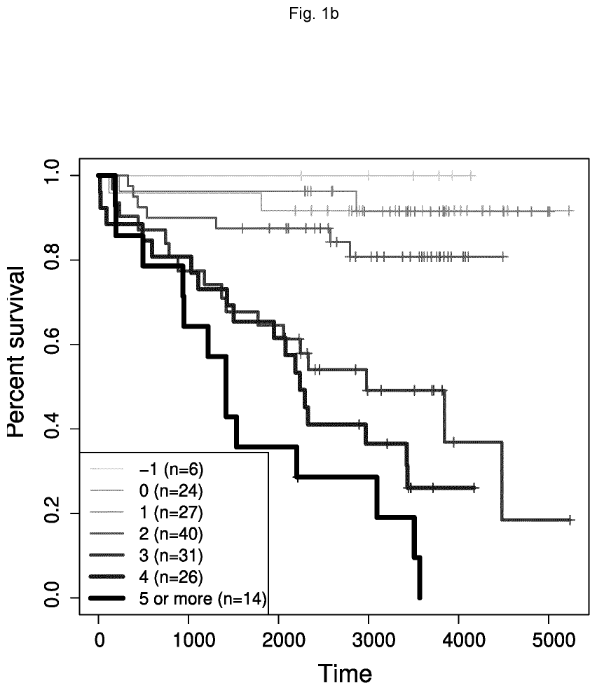

As shown in the examples and in particular in FIG. 1 a, TcGTs are directly correlated with a decrease in survival in a cumulative way. This happens, either by accumulating the presence of predictors of poor survival (harmful predictor), or the absence of predictors of good survival (protective predictor). In other words, a subject that suffering from cancer that has only 1 harmful event (e.g. gain of 2 harmful predictor and loss of 1 protective predictor) has much better prognosis than a patient that has 3 harmful events.

Preferably, the at least one TE is selected from the non-limiting group comprising AluJr, HAL1, L1PA16, AluSx, L1PA8, L1PA5, L1PA8, L1PA7, AluJr, L1PA7, MIR, L2b, L2c, M1Rb, LTR16A, THE1D, MLT2B4, ERVL-B4-int, LTR12, MIR3, L1PA3, L1PA2, L2b, LTR33, LTR66, L2d2, AluSg7 and AluSx, a fragment or variant thereof, or a combination of one or more thereof.

As shown in the examples and in the corresponding FIG. 1a, 300 patients have been classified considering their expression of TcGTs in primary colorectal cancer tumor, listed in Table 1. For every patient, the number of TcGTs correlating with an increase of survival (protective) minus the ones correlated with a decrease of survival (harmful) has been computed. Patients can have either the same number of protective and harmful events (1), more harmful than protective (1, 2, 3, 4, and 5) or more protective than harmful (−1) (FIG. 1a).

A combination of these TcGTs (Table 1 and FIG. 1a) builds a fingerprint (i.e. a signature) which is a stronger predictor of survival than any of the TcGTs alone (FIG. 1b). The accumulation of expressed harmful predictors and the concomitant loss of expressed protective predictors in a patient suffering from colorectal cancer leads to a remarkably lower survival time (FIG. 1b).

The present invention further contemplates, a method for staging a cancer in a subject, the method comprising measuring, directly or indirectly, in a biological sample of said subject, the level of expression or activity of at least one nucleic acid sequence and comparing said level of expression or activity to a control value or range, wherein said at least one nucleic acid sequence comprises

i) at least one transposable element (TE), a fragment or variant thereof, and
ii) at least one gene sequence, a fragment or variant thereof.

Preferably, the at least one nucleic acid is selected from the group listed in Table 1, or a variant or fragment thereof.

Preferably, the gene sequence is a POU5F1B sequence, fragment or variant thereof and the cancer is a solid tumor cancer, preferably colorectal cancer. Also preferably, the TE is selected from the non-limiting group comprising LTR66, LTR33, MIR, L2a, L2b, L2c and MIRb, a variant, a fragment or a combination thereof. Most preferably, the TE is LTR66, a variant, a fragment or a combination of one or more thereof.

As shown in the examples, the expression of the LTR66-POU5F1B TcGT tightly correlates with the transition from adenoma to carcinoma in particular from stages I to IV of colorectal cancer.

The present invention also concerns a method for treating and/or preventing cancer and/or cancer metastasis in a subject in need thereof, said method comprising administering an inhibitor of the invention.

As used herein, the term “treatment” or “treating” means any administration of a composition, pharmaceutical composition, therapeutic agent, compound, etc. of the disclosure to a subject for the purpose of:
(i) inhibiting the disease, that is, arresting the development of clinical symptoms; and/or
(ii) relieving the disease, that is, causing the regression of clinical symptoms.

As used herein, the term “prevention” or “preventing” means any administration of a composition, pharmaceutical composition, therapeutic agent, compound, etc. of the disclosure to a subject for the purpose of preventing the disease, that is, causing the clinical symptoms of the disease not to develop.

In the context of the present invention, the disease is cancer, preferably a solid tumor cancer. More preferably, the solid tumor cancer is selected from the non-limiting group comprising Colorectal Cancer, Gastric Cancer, Prostate Cancer, Liver Cancer, Testis Cancer, Ovary Cancer and Breast Cancer, or a combination of one of more of these cancers. Even more preferably, the cancer is Colorectal Cancer.

The present invention also contemplates an inhibitor of at least one i) nucleic acid sequence of the invention or ii) peptide or protein of the invention for use in the treatment and/or prevention of cancer and/or cancer metastasis.

In one aspect of the present invention, the inhibitor according to the present invention is typically a nucleic acid molecule comprising and/or encoding an oligonucleotide with the reverse complement sequence of the nucleic acid sequence (e.g. RNA) it inhibits. The inhibitor hybridizes to the nucleic acid sequence thereby negating or impairing its activity. The inhibitor typically comprises one or more modifications to enhance the hybridization of the inhibitor to the target nucleic acid sequence. A nucleic acid chemical analog preferably comprises an RNA backbone, preferably with one or more modification such as a 2-0′-methyl modification, a locked nucleic acid (LNA) modification, a morpholino modification and/or a peptide nucleic acid (PNA) modification. The modifications can be at one or more sugar moieties of the backbone. Next to these chemical modifications, it has been shown that adding flanking sequences to the antisense-based RNA inhibitors strongly enhances the inhibitory potency of these molecules. Preferably, these flanking sequences form stem-loop hairpin structures with a total of between 8 and 16 bases on both 5′ and 3′ end of the antisense oligomer. Most preferred are flanking sequences of 12 bases, with a 4 base pair (2×4 bases) stem region and a loop region of 4 bases. These added structural elements are thought to enhance and stabilize the interaction between the RNA-RISC complex and the inhibitor, thus prolonging the effect of the inhibitor.

A preferred inhibitor of at least one acid nucleic of the invention is an inhibitor selected from the group comprising an siRNA, an shRNA, an snRNA, a siRNA capable of interfering the expression of short hairpin (sh), apiRNA, a nucleic acid including an antisense oligonucleotide (e.g. ASOs, modified ASOs such as GapmeRs, . . . ) or nucleic acid chemical analog capable of interfering with the activity or expression of one or more nucleic acid sequence (e.g. RNA), a small RNA zipper and a morpholino antisense oligonucleotide capable of interfering with the expression of one or more nucleic acid sequence. Preferably, the inhibitor of at least one acid nucleic of the invention is an oligonucleotide that interferes with the activity or expression of the nucleic acid sequence (e.g. RNA) of the invention by hybridizing to the nucleic acid sequence of the corresponding nucleic acid sequence or to a variant or fragment thereof.

Alternatively, the inhibitor is a single-stranded RNA molecule complementary to the specific nucleic acid sequence (e.g. RNA) of the invention, or to a variant or fragment thereof and that is conjugated to cholesterol.

The terms “siRNA” and “short interfering RNA” as used herein are interchangeable and refer to single-stranded or double-stranded RNA molecules that are capable of inducing RNA interference. SiRNA molecules typically have a duplex region that is between 18 and 30 base pairs in length.

The terms “piRNA” and “Piwi-interacting RNA” are interchangeable and refer to a class of small RNAs involved in gene silencing. PiRNA molecules typically are between 26 and 31 nucleotides in length.

The terms “shRNA” as used herein refers to a nucleic acid molecule comprising at least two complementary portions hybridized or capable of specifically hybridizing to form a duplex structure sufficiently long to mediate RNAi (typically between 15-29 nucleotides in length), and at least one single-stranded portion, typically between approximately 1 and 10 nucleotides in length that forms a loop connecting the ends of the two sequences that form the duplex. Exemplary shRNA of the invention may, e.g. be selected from the group non-limiting group comprising the shRNA3 (5′-CACAGGTGATTATGATTTAAA-3′, SEQ ID No. 28) and the shRNA5 (5′-CACATTCAGTCAACATTTA-3′, SEQ ID No. 29) that were designed to be uniquely and selectively recognizing the 5′UTR of POU5F1B gene.

The terms “snRNA” and “small nuclear RNA” are interchangeable and refer to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs comprising antisense sequences directed against one or more acid nucleic sequence of the invention.

Non-limiting examples of modified ASOs include the GapmeRs. As used herein, a GapmeR is a chimeric antisense oligonucleotide that contains a central block of deoxynucleotide monomers sufficiently long to induce RNase H cleavage. Usually, the GapmeRs of the invention are directed against one or more acid nucleic sequences of the invention.

An inhibitor of at least one acid nucleic of the invention as well as a variant or fragment thereof may be prepared by recombinant techniques using either prokaryotic or eukaryotic host cells. Alternatively, the inhibitor may be synthesized using commercially available synthesizers in known ways.

Additional considerations can be taken into account when designing antisense oligonucleotides, including: (i) sufficient specificity in binding to the target sequence; (ii) solubility; (iii) stability against intra- and extracellular nucleases; (iv) ability to penetrate the cell membrane; and (v) when used to treat an organism, low toxicity.

Preferably, the inhibitor of at least one acid nucleic of the invention is capable of interfering with the activity or expression of a nucleic acid sequence of the invention, preferably a nucleic acid sequence selected from the group comprising SEQ ID No. 1, SEQ ID No. 2, SEQ ID No. 3, SEQ ID No. 4, SEQ ID No. 5, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, a variant, a fragment or a combination thereof.

More preferably, the inhibitor of at least one acid nucleic of the invention is capable of interfering with the activity or expression of a nucleic acid sequence selected from the group comprising SEQ ID No. 1, SEQ ID No. 2, SEQ ID No. 3, SEQ ID No. 4 and SEQ ID No. 5, a variant, a fragment or a combination thereof.

In another aspect of the invention, the inhibitor is a chemical agent, oligonucleotide, antibody, peptide or an antigen-binding fragment of said antibody that interferes i) with the expression of a target gene of the nucleic acid sequence of the invention, ii) by specifically binding to a nucleic acid sequence of the invention or to a polypeptide encoded by a nucleic acid sequence of the invention, or by iii) inducing selective intracellular proteolysis of a peptide encoded by a nucleic acid sequence of the invention.

As used herein, an “antibody” is a protein molecule that reacts with a specific antigenic determinant or epitope and belongs to one or five distinct classes based on structural properties: IgA, IgD, IgE, IgG and IgM. The antibody may be a polyclonal (e.g. a polyclonal serum) or a monoclonal antibody, including but not limited to fully assembled antibody, single chain antibody, antibody fragment, and chimeric antibody, humanized antibody as long as these molecules are still biologically active and still bind to at least one peptide of the invention. Preferably the antibody is a monoclonal antibody. Preferably also the monoclonal antibody will be selected from the group comprising the IgG1, IgG2, IgG2a, IgG2b, IgG3 and IgG4 or a combination thereof. Most preferably, the monoclonal antibody is selected from the group comprising the IgG1, IgG2, IgG2a, and IgG2b, or a combination thereof.

An “antigen binding fragment” comprises a portion of a full-length antibody. Examples of antigen binding fragments include Fab, Fab′, F(ab′)2, and Fv fragments; diabodies; linear antibodies; single-chain antibody molecules; and multispecific antibodies formed from antibody fragments.

As used herein, a “chemical agent” is a compound that produces change by virtue of its chemical composition and its effects on living tissues and organisms. The chemical agent may be a small molecule inhibitor (SMI), most preferably a non-peptidyl SMI, that is preferably inducing selective intracellular proteolysis of a peptide encoded by a nucleic acid sequence of the invention. Examples of chemical compounds inducing selective intracellular proteolysis comprise proteolysis targeting chimera (PROTAC) protein degraders and small-molecule chemical modulators of deubiquitinating enzymes upstream of or on the proteasome. As known in the art, PROTAC is a heterobifunctional small molecule composed of two active domains and a linker capable of removing specific unwanted proteins.

Also contemplated in the present invention is a pharmaceutical composition comprising a therapeutically effective amount of an inhibitor of the invention as described herein and a pharmaceutically acceptable carrier or diluent.

Usually, the pharmaceutical composition of the invention is for use in the treatment and/or prevention of cancer and/or cancer metastasis.

The term “therapeutically effective amount” as used herein means an amount of inhibitor of one or more nucleic acid sequence associated with cancer high enough to significantly positively modify the symptoms and/or condition to be treated, but low enough to avoid serious side effects (at a reasonable risk/benefit ratio), within the scope of sound medical judgment. The therapeutically effective amount of the inhibitor of one or more nucleic acid sequence associated with cancer is selected in accordance with a variety of factors including type, species, age, weight, sex and medical condition of the patient; the severity of the condition to be treated; the route of administration; the renal and hepatic function of the patient. A physician of ordinary skill in the art can readily determine and prescribe the effective amount of the drug required to prevent, counter or arrest the progress of the cancer.

“Pharmaceutically acceptable carrier or diluent” means a carrier or diluent that is useful in preparing a pharmaceutical composition that is generally safe, non-toxic, and desirable, and includes carriers or diluents that are acceptable for human pharmaceutical use. Such pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Pharmaceutically acceptable excipients include starch, glucose, lactose, sucrose, sodium stearate, glycerol monostearate, talc, sodium chloride, dried skim milk, glycerol, propylene glycol, water, ethanol and the like.

The pharmaceutical compositions may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include macrocrystalline cellulose, carboxymethyf cellulose sodium, polysorbate 80, phenyletbyl alcohol, chiorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

The pharmaceutical composition can also further comprise at least one additional anticancer agent or therapy selected from the group comprising radiotherapy, chemotherapy, immunotherapy and hormone therapy, or a combination of one of more thereof.

Further contemplated is a host cell as well as a pharmaceutical composition comprising a therapeutically effective amount of said host cell (e.g. single cell or population of cells) and a pharmaceutically acceptable carrier or diluent. Preferably, the host cell, comprises or has been modified by the introduction of i) a gene delivery vector comprising one or more nucleic acid(s) encoding the inhibitor of at least one acid nucleic of the invention or ii) one or more nucleic acid(s) encoding the inhibitor of at least one acid nucleic of the invention.

The pharmaceutical composition comprising a therapeutically effective amount of a host cell and a pharmaceutically acceptable carrier or diluent can be administered to the subject in need thereof by any method and route known in the art and described herein.

The present invention further contemplates a method of treatment of cancer, and/or cancer metastasis comprising:

(a) providing an inhibitor of the invention and
(b) administering said inhibitor to a subject suffering from cancer.

Preferably, said inhibitor is in the form of a pharmaceutical composition comprising a therapeutically effective amount of said inhibitor as described herein.

Alternatively, the method of treatment can be associated with a treatment selected from the group comprising radiotherapy, chemotherapy, immunotherapy or hormone therapy.

“Administering”, as it applies in the present invention, refers to contact of a therapeutically effective amount of an inhibitor of the invention, to the subject.

In general, methods of administering a composition comprising an inhibitor in the form of nucleic acids are well known in the art. In particular, the routes of administration already in use for nucleic acid therapeutics, along with formulations in current use, provide preferred routes of administration and formulation for the nucleic acids described herein.

Nucleic acid compositions can be administered by a number of routes including, but not limited to: oral, intravenous, intraperitoneal, intramuscular, transdermal, subcutaneous, topical, sublingual, or rectal means. Nucleic acids can also be administered via liposomes or nanoparticles. Such administration routes and appropriate formulations are generally known to those of skill in the art.

Administration of the formulations (e.g. pharmaceutical compositions) described herein may be accomplished by any acceptable method which allows the inhibitor of the invention or the nucleic acid encoding the inhibitor of at least one acid nucleic of the invention to reach its target. The particular mode selected will depend of course, upon factors such as the particular formulation, the severity of the state of the subject being treated, and the dosage required for therapeutic efficacy. The actual effective amounts of drug can vary according to the specific drug or combination thereof being utilized, the particular composition formulated, the mode of administration, and the age, weight, condition of the patient, and severity of the symptoms or condition being treated.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. The composition can be injected intradermally for treating cancer, for example.

The injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

Preferably, the agent and/or nucleic acid delivery system are provided in a manner which enables tissue-specific uptake of the agent and/or nucleic acid delivery system. Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

The formulations may be delivered using a bio-erodible implant by way of diffusion or by degradation of the polymeric matrix.

The administration of the formulation may be designed so as to result in sequential exposures to the inhibitor of the invention over a certain time period, for example, hours, days, weeks, months or years. This may be accomplished, for example, by repeated administrations of a formulation or by a sustained or controlled release delivery system in which the inhibitor is delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems suitable include, but are not limited to, time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include nonpolymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include, but are not limited to, erosional systems in which the miRNA inhibitor is contained in a formulation within a matrix (for example, as described in U.S. Pat. Nos. 4,452,775, 4,675,189, 5,736,152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. The system may allow sustained or controlled release of the composition to occur, for example, through control of the diffusion or erosion/degradation rate of the formulation containing the miRNA inhibitor. In addition, a pump-based hardware delivery system may be used for delivery.

Also envisioned is a tumour-specific delivery of an inhibitor-coupled super-paramagnetic iron oxide nanoparticles as known in the art.

The present invention also contemplates a gene delivery vector, preferably in the form of a plasmid or a vector, that comprises one or more nucleic acid(s) encoding an inhibitor of at least one nucleic acid of the invention. Preferably, said inhibitor is selected from the group comprising an siRNA, an shRNA, an snRNA, a siRNA capable of interfering the expression of short hairpin (sh), apiRNA, a nucleic acid including an antisense oligonucleotide or a combination of one or more thereof. Most preferably, the nucleic acid including an antisense oligonucleotide is selected from the group comprising an antisense oligonucleotide (ASO) and a modified ASO.

As used herein, a “vector” is capable of transferring nucleic acid sequences to target cells (e.g., viral vectors, non-viral vectors, particulate carriers, and liposomes).

Suitable vectors include derivatives of SV40 and known bacterial plasmids, e. g., E. coli plasmids col El, pCR1, pBR322, pMB9 and their derivatives, plasmids such as RP4; phage DNAs, e. g., the numerous derivatives of phage X, e. g., NM989, and other phage DNA, e. g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Various viral vectors are used for delivering nucleic acid to cells in vitro or in vivo. Non-limiting examples are vectors based on Herpes Viruses, Pox-viruses, Adeno-associated virus, Lentivirus, and others. In principle, all of them are suited to deliver the expression cassette comprising an expressible nucleic acid molecule that codes for an inhibitor of at least one nucleic acid of the invention. In a preferred aspect, said viral vector is an adenoviral vector, preferably a replication competent adenovirus.

The present invention further contemplates a method of treatment of cancer, and/or cancer metastasis, comprising modifying a host cell, and reintroducing the host cell (e.g. single cell or population of cells) into the patient in need thereof, i.e. suffering from cancer and/or cancer metastasis.

Preferably, a biopsy or other tissue or biological fluid sample comprising the single cell or the population of cells may be necessary. Cells such as fibroblast cells or stem cells that can be generated directly from adult cells, such as iPSCs, are particularly preferred in this regard.

A gene delivery vector (e.g. plasmid or vector) comprising one or more nucleic acid(s) encoding an inhibitor of at least one nucleic acid of the invention or one or more nucleic acid(s) encoding an inhibitor of at least one acid nucleic of the invention can be introduced to host cell via one or more methods known in the art. These one or more methods include, without limitation, microinjection, electroporation, calcium phosphate-mediated transfection, cationic transfection, liposome transfection, dendrimer transfection, heat shock transfection, nucleofection transfection, magnetofection, lipofection, optical transfection, proprietary agent-enhanced uptake of nucleic acids, and delivery via liposomes, immunoliposomes, virosomes, or artificial virions.

It will be appreciated that in the present method the modification following the introduction of the gene delivery vector (plasmid or vector), or the one or more nucleic acid(s) encoding the inhibitor of at least one acid nucleic of the invention, to the host cell may occur ex vivo or in vitro, for instance in a cell culture and in some instances not in vivo. In other aspects, it may occur in vivo.

The host cell(s) (e.g. single cell or population of cells) of the invention is then reintroduced into the patient in need thereof by any route of administration and/or delivery methods known in the art, as described herein.

The invention also contemplates kits for the treatment and/or prevention of cancer and/or cancer metastasis. In one aspect of the invention, the kit comprises a pharmaceutical composition comprising an inhibitor of the invention.

In a further aspect, the invention contemplates a kit for the treatment and/or prevention of cancer and/or cancer metastasis comprising a host cell of the invention.

The kits of the invention may also comprise a container and a label or package insert on or associated with the container. Suitable containers include, for example, bottles, vials, syringes, etc. The containers may be formed from a variety of materials such as glass or plastic. The container holds a composition which is effective for treating the disease of disorder of the invention and may have a sterile access port (for example the container may be an intravenous solution bag or a vial having a stopper pierceable by a hypodermic injection needle). Alternatively, or additionally, the kits may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The label or package insert may comprise instructions for use thereof. Instructions included may be affixed to packaging material or may be included as a package insert. While the instructions are typically written or printed materials they are not limited to such. Any medium capable of storing such instructions and communicating them to an end user is contemplated by this disclosure.

Further contemplated in the present invention are nucleic acids encoding an siRNA, an shRNA, an snRNA, a siRNA capable of interfering the expression of short hairpin (sh), a piRNA, a nucleic acid including an antisense oligonucleotide (e.g. ASOs, modified ASOs such as GapmeRs, . . . ) of the invention.

Also contemplated is a nucleic acid sequence comprising at least one acid nucleic sequence of the invention. Preferably, the nucleic acid sequence is an RNA transcript, a cDNA of said RNA transcript or a DNA sequence. Most preferably, the at least one acid nucleic sequence of the invention is selected from the group comprising SEQ ID No 1, 2, 3, 4, 5, 6, 7, 8, 9, 10, 11, 12, 13, 14, 15, 16, 17, 18, 19, 20, 21, 22, 23, 24, 25, 26, and 27, a fragment or variant thereof or a combination of one or more thereof.

It is also understood that the methods of the invention also comprise detecting the presence or absence of at least one polypeptide encoded by a nucleic acid sequence of the invention, a fragment or variant thereof, in a biological sample. Detection of said at least one one polypeptide can be direct or indirect and determined by any suitable method known in the art (e.g Western blots, ELISA, HPLC, LC/MS, . . . ).

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein. Various references are cited throughout this Specification, each of which is incorporated herein by reference in its entirety. The foregoing description will be more fully understood with reference to the following Examples.

### EXAMPLES

### Example 1

### Material and Methods

### Primary Tumors Data

The first dataset analyzed here is a publically available RNA-seq under the GEO accession number GSE50760. It includes RNA-seq data from 54 samples (normal colon, primary CRC, and liver metastasis) from 18 CRC patients.

The second RNA-seq and related clinical data was developed by Professor Claus Lindbjerg Andersen at the Department of Molecular Medicine at Aarhus University Hospital-Skejby, in Denmark. It contains RNA-seq and clinical data from paired tissue biopsies (tumor and normal mucosa) obtained from 301 patients, including 33 adenoma and 281 carcinoma samples, and 301 normal mucosa samples.

### RNA-Seq Analysis

RNA-Seq reads were aligned to the hg19 genome assembly using HISAT2^{[1]} (with parameters—rna-strandness R). Reads that were not uniquely mapped were discarded from the analysis using bamtools filter v2.4.1 with parameters -tag “NH:1”. Read summarization on genes and TEs were generated using featureCounts^{[2]} with parameters -s 2 -t exon -g gene_id-Q 10. Sequencing depth normalization was performed using the TMM method as implemented in the voom function from the R package LIMMA from Bioconductor_{[3]}. The gene library sizes as given by voom were used to normalize the TEs counts.

### Transpochimeric Transcripts Analysis

For each sample, transcripts with reads spanning between genomic positions annotated as genes and TE loci were extracted from the aligned RNA-seq file. Genomic positions for genes were obtained from the ENSEMBL database for hg19 (GRCh37). TE loci were obtained from RepBase. Only transcripts, starting at a TE which was not overlapping an annotated gene exon, were considered.

### Cell Culture

The cell lines used in this study were purchased from the American Type Culture Collection (HT29, SW620 and LS1034). All cell lines were cultured with their corresponding medium supplemented with 10% fetal calf serum at 37° C. and 5% CO₂. HT29 cell line was cultured in McCoy's 5A medium; SW620 in Leibovitz's L-15 medium; and LS1034 in RPMI medium. Cells were transduced, selected with 2 ug/mL puromycin and collected after 5 days for the colony formation assays experiments.

### Plasmids and Lentiviral Vectors

pLKO.puro shRNA vectors were used for POU5F1B knock down. The shRNA3 (5′-CACAGGTGATTATGATTTAAA-3′) and the shRNA5 (5′-CACATTCAGTCAACATTTA-3′) were designed to be uniquely matching the 5′UTR of POU5F1B gene. pTRE.GFP.puro and pTRE.POU5F1B.puro vectors were used for overexpression of GFP and POU5F1B proteins respectively. Lentiviral vectors production protocols are detailed at www.tronolab.epfl.ch and backbones are available at Addgene (www.addgene.org).

### Soft Agar Colony Formation Assays

A soft agar colony formation assay of suspension cells was performed in 6-well culture plates. In short, 20,000 cells were mixed in 0.37% low-gelling agar (Sigma)/medium and layered on top of a base layer of 0.7% low-gelling agar (Sigma)/medium. A layer of RPMI 10% FBS was settled on top to avoid agar drying and to allow cell feeding for 3 weeks. Cells were MTT-stained and colonies were quantified using Fiji software.

### Results

Colorectal cancer (CRC) has been the focus of our studies because its treatment is an unmet clinical need and its step-wise development has been extremely well documented. Furthermore, samples of tumor and healthy tissues are systematically obtained during surgery. We first analyzed a publicly available RNA-Seq dataset (GSE50760) comprising normal colon, primary tumor and liver metastasis from 18 CRC patients, searching for CRC-specific TcGTs. We found a very interesting candidate which was expressed in 0 out of 18 normal samples; 10 out of 18 primary CRC samples; and 14 out of 18 liver metastases. This transcript is encoded on chromosome 8 and initiates within an LTR66 to terminate at the 3′end of the POU5F1B gene, 170 kb downstream.

In order to validate our findings and link TcGTs expression to survival, we analyzed another RNA-seq dataset comprising paired normal-primary tumor samples from 300 CRC patients and their survival data. We examined the non-annotated TcGTs that met the following criteria: they should start on a TE; this TE should not overlap any annotated coding exon; and they had to be expressed in more than 1% of the total number of samples. Then we fitted an additive Cox proportional hazards regression model regularized by an elastic net penalty (using the coxnet implementation) to determine which TcGTs were better at predicting survival of CRC patients. We ended up with a list of TcGTs that predict survival in CRC patients if expressed in primary tumors (Table 1). Notably, some TcGTs are predictors of better survival (protective, indicated as (+) in Table 1, while some others are predictors of poorer survival (harmful, indicated as (−) in Table 1).

TABLE 1
The list of 27 TcGTs expressed in primary colorectal cancer (CRC) that implicate
the coding sequence of 10 different genes and that predict survival in CRC
patients. All the TcGTs indicated as (+) are predictors of better survival,
while the ones indicated as (−) are predictors of poorer survival.

| | | | | | | SEQ ID | |
|---|---|---|---|---|---|---|---|
| | | | | | TE in first | No. | |
| Chr | Start | End | Strand | Gene | exon | (cDNA) | Predictor |
| chr5 | 112316746 | 112320349 | + | DCP2 | AluJr4\|HAL1 | 9 | + |
| chrX | 57372226 | 57475153 | + | FAAH2 | L1PA16 | 10 | − |
| chrX | 57484988 | 57515626 | + | FAAH2 | AluSx\|L1PA8 | 11 | − |
| chrX | 57506679 | 57515626 | + | FAAH2 | L1PA5 | 12 | − |
| chrX | 57508024 | 57515617 | + | FAAH2 | L1PA8 | 13 | − |
| chr2 | 149070638 | 149220250 | + | MBD5 | L1PA7 | 14 | − |
| chr2 | 149197378 | 149221463 | + | MBD5 | AluJr | 15 | − |
| chr2 | 149067806 | 149228031 | + | MBD5 | L1PA7 | 16 | − |
| chr2 | 149098570 | 149228861 | + | MBD5 | MIR | 17 | − |
| chr11 | 86152121 | 86173689 | − | ME3 | L2b | 18 | − |
| chr11 | 86152097 | 86222190 | − | ME3 | L2c | 19 | − |
| chr11 | 86152213 | 86214317 | − | ME3 | MIR\|LTR16A | 20 | − |
| chr11 | 86156551 | 86380561 | − | ME3 | THE1D | 21 | − |
| chr11 | 114447900 | 114519204 | − | NXPE4 | MLT2B4\|ERV | 22 | − |
| | | | | | L-B4-int | | |
| chr11 | 114441314 | 114522320 | − | NXPE4 | LTR12 | 23 | − |
| chr1 | 201261398 | 201287858 | + | PKP1 | MIR3 | 24 | − |
| chr20 | 8095137 | 8633940 | + | PLCB1 | L1PA3 | 25 | − |
| chr20 | 8581327 | 8665721 | + | PLCB1 | L1PA2 | 26 | − |
| chr8 | 128420979 | 128429391 | + | POU5F1B | MIRb | 1 | − |
| chr8 | 128415837 | 128429403 | + | POU5F1B | L2c | 2 | − |
| chr8 | 128411137 | 128429310 | + | POU5F1B | L2b | 3 | − |
| chr8 | 128351525 | 128429402 | + | POU5F1B | LTR33 | 4 | − |
| chr8 | 128256705 | 128429509 | + | POU5F1B | LTR66\|LTR66 | 5 | − |
| chr15 | 64406806 | 64431545 | + | SNX1 | L2d2 | 27 | − |
| chr2 | 32521449 | 32537385 | + | YIPF4 | AluSg7 | 6 | + |
| chr2 | 32471200 | 32537258 | + | YIPF4 | AluSx | 7 | + |
| chr2 | 32442175 | 32537286 | + | YIPF4 | AluSx | 8 | + |

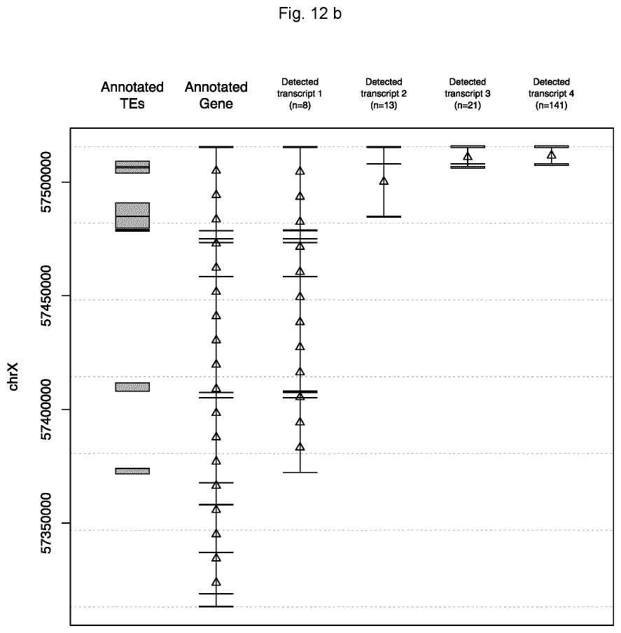

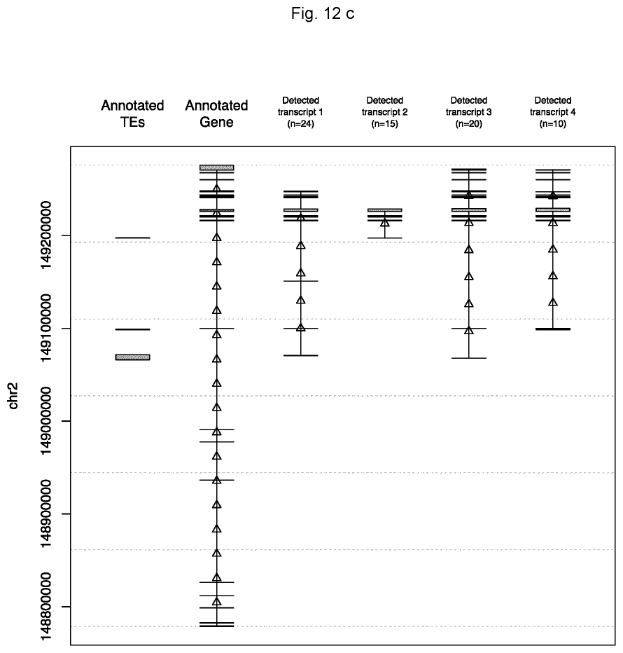

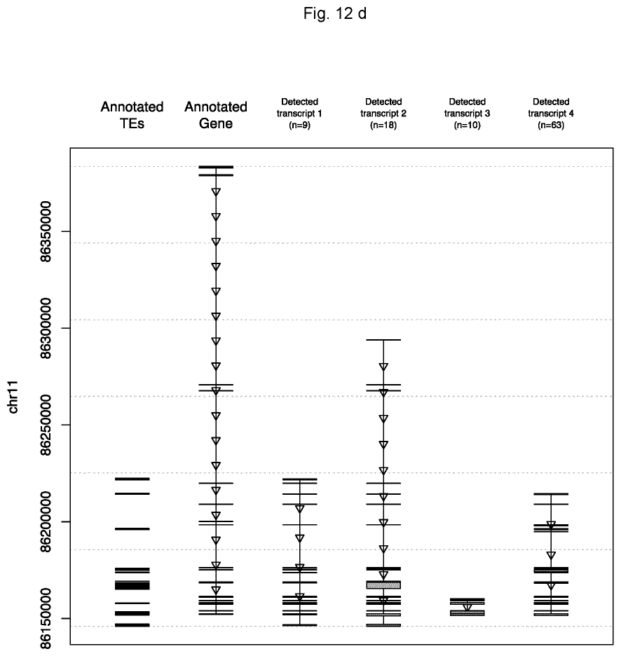

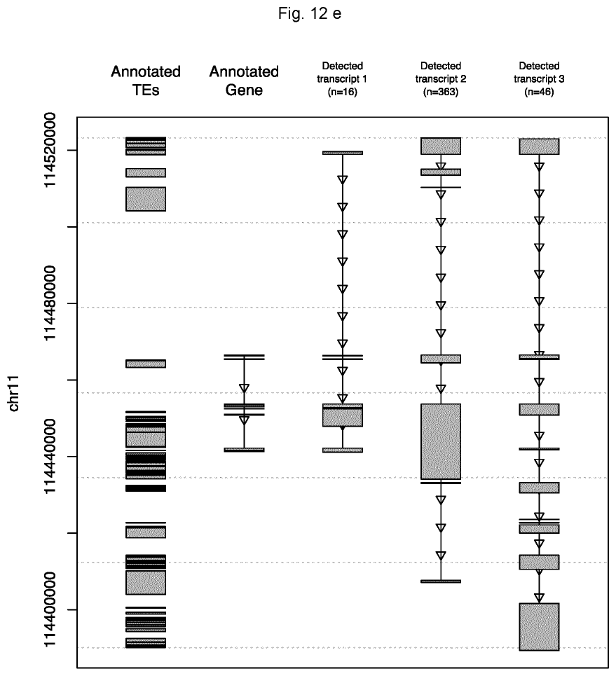

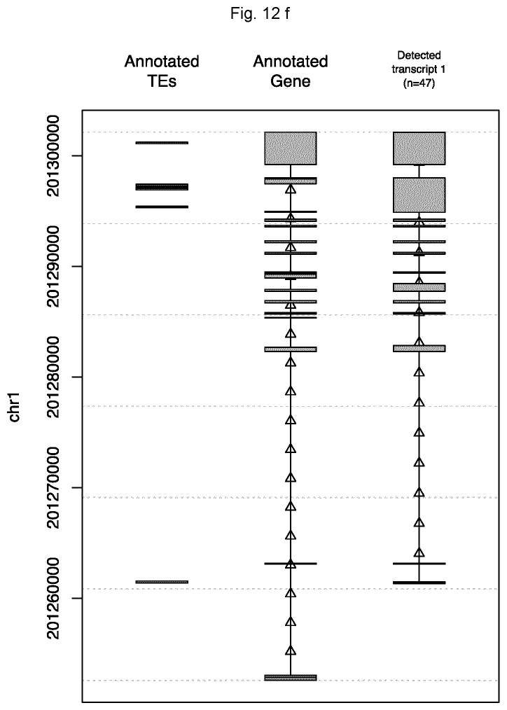

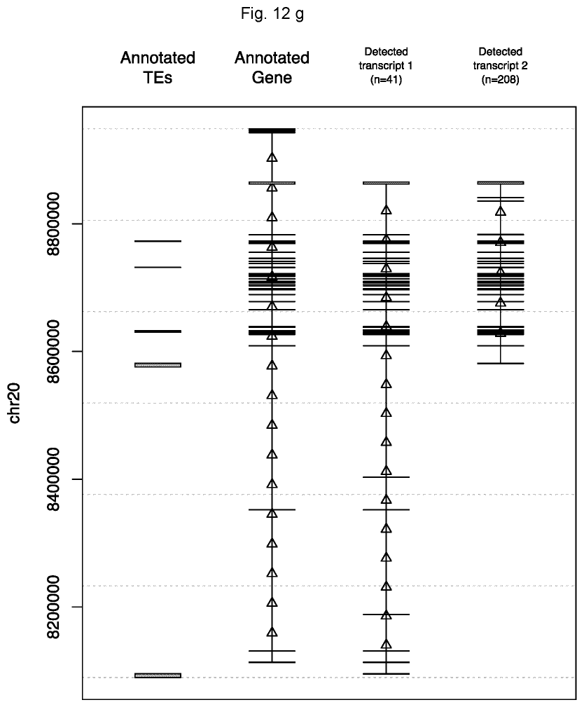

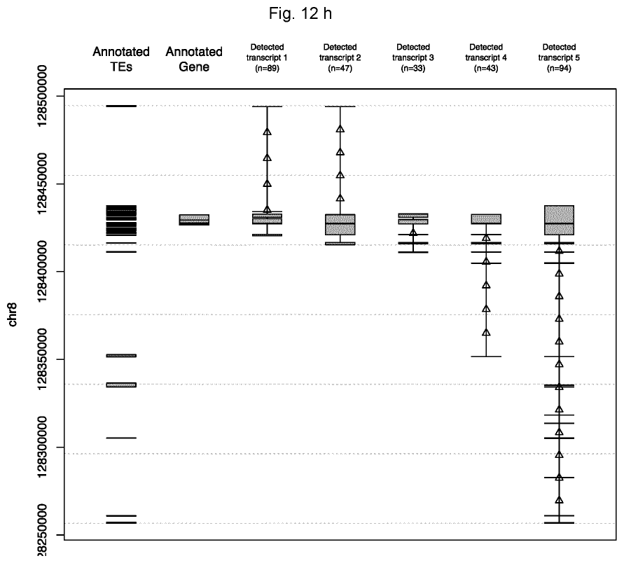

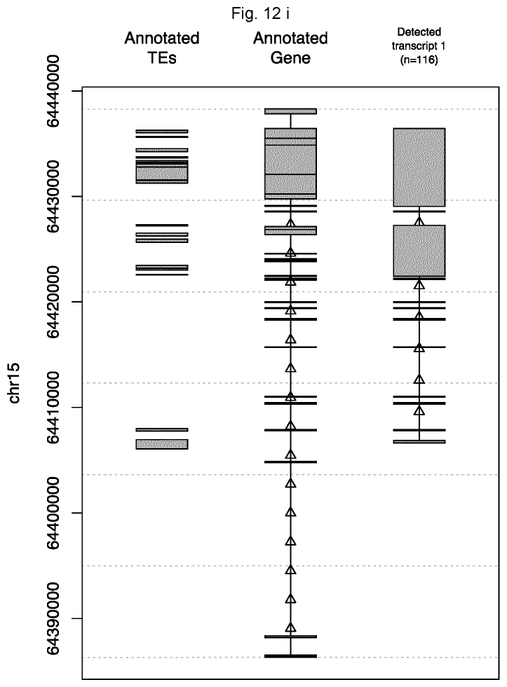

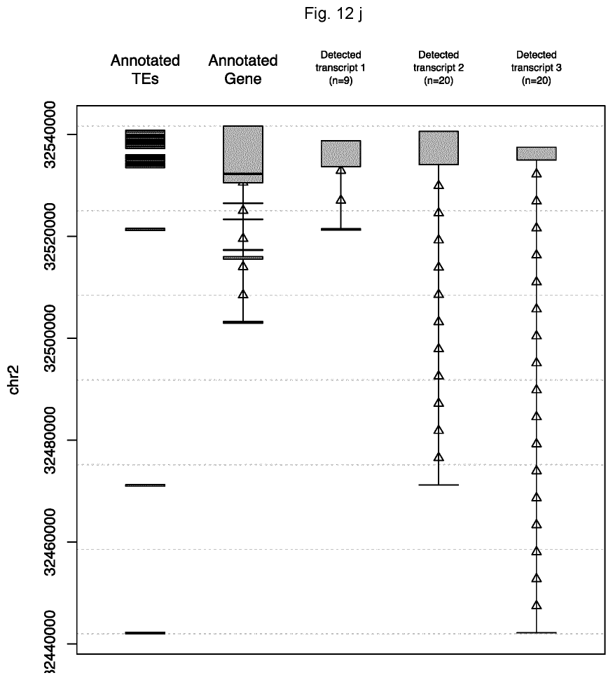

A combination of these TcGTs (Table 1, FIG. 1a, and FIG. 12) builds a fingerprint which is a stronger predictor of survival than any of the TcGTs alone (FIG. 1b). The accumulation of expressed harmful predictors and the concomitant loss of expressed protective predictors in a CRC patient leads to a remarkably lower survival time (FIG. 1b).

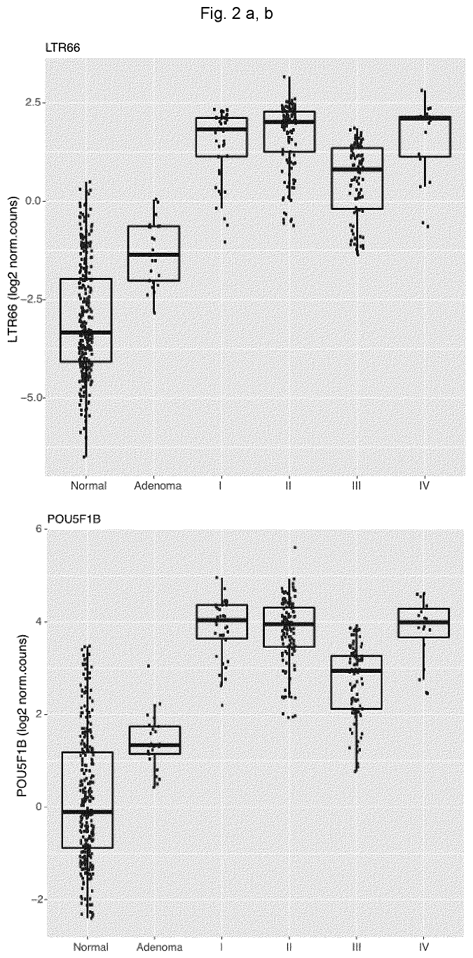

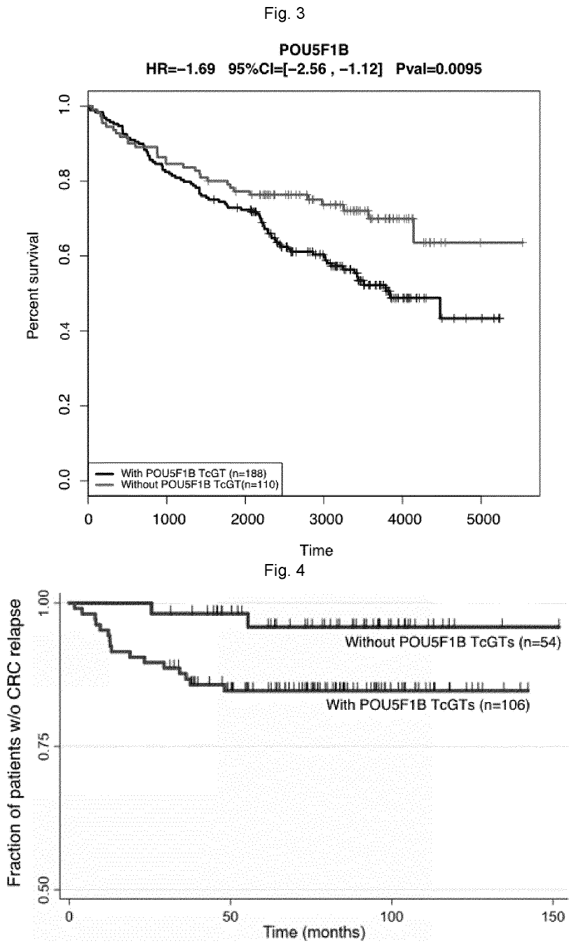

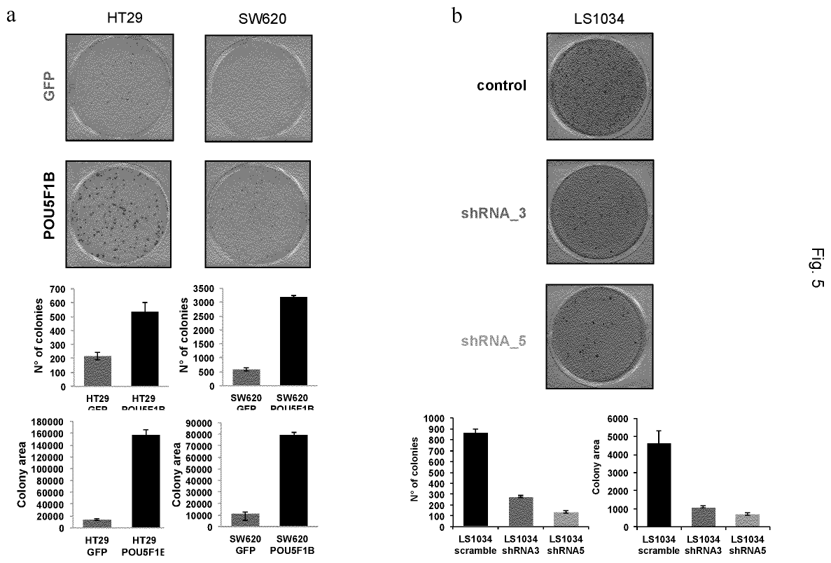

The Inventors found that the 5 different TcGTs encoding for POU5F1B displayed very striking numbers, being expressed in 2 out of 301 normal vs. 196 out of 313 primary CRC samples. The LTR66-POU5F1B TcGT, previously found in the GSE50760 RNA-seq dataset, is the longest of these 5 transcripts and it is the more frequent within CRC samples. Expression of the LTR66-POU5F1B transcript (SEQ ID No. 5) tightly correlates with the transition from adenoma (a benign tumor) to carcinoma (its malignant counterpart), which points out its potential application as a biomarker for CRC staging (FIG. 2). Moreover, the presence of the 5 TcGTs of POU5F1B in CRC is linked to a lower survival time (FIG. 3) and to a decrease in relapse-free survival (FIG. 4).

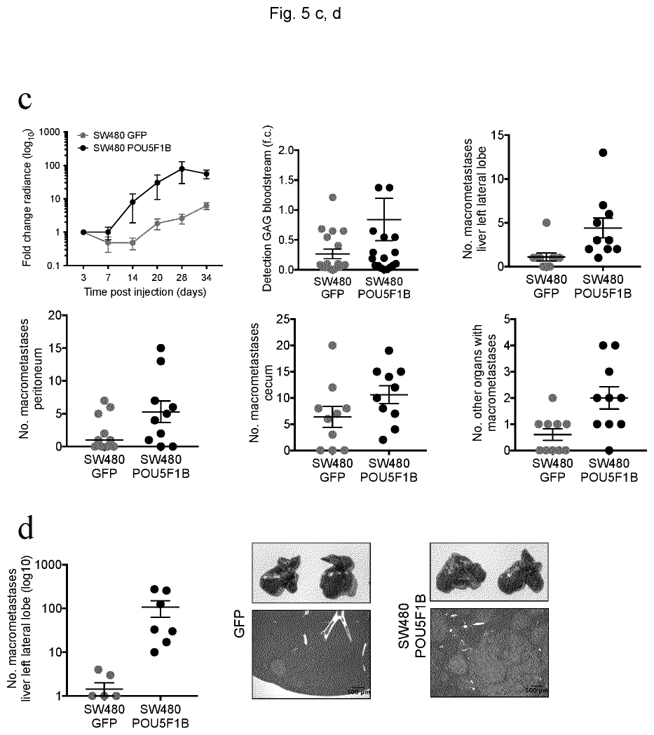

The POU5F1B gene has been linked to an aggressive phenotype in gastric cancer^{[5]} and to prostate cancer susceptibility^{[6]}, has been proposed to promote hepatocellular carcinoma cells proliferation by activating AKT^{[7]} and is overexpressed in some human breast cancer^{[8]}. However, the POU5F1B gene has never been linked to colorectal cancer and, to our knowledge, no POU5F1B-related TcGT has been previously described. Our first approach for testing the impact of POU5F1B protein in CRC was to perform colony formation assays in the POU5F1B-transduced HT29 and SW620 CRC cell lines (FIG. 5a); and in the POU5F1B-knocked down LS1034 CRC cell line (FIG. 5b). This experiment confirmed an increase of the clonogenicity capacity in the CRC cell lines overexpressing POU5F1B protein, and the inverse phenotype in the POU5F1B-knock down conditions (FIG. 5).

In vivo monitoring of orthotopically-engrafted mice by bioluminescence showed higher tumor growth rates in SW480-POU5F1B versus SW480-GFP-engrafted animals (FIG. 5c). SW480-POU5F1B-engrafted animals presented higher number of circulating tumor cells (CTCs), number of macrometastases in the liver left lateral lobe, in the peritoneum, in the cecum, and higher number of other organs with macrometastases (FIG. 5c). An additional model of metastasis—intrasplenic injection of CRC cells—showed that SW480-POU5F1B cells generated more liver metastases than SW480-GFP cells (FIG. 5d). Altogether, these results suggest the role of POU5F1B in CRC metastasis.

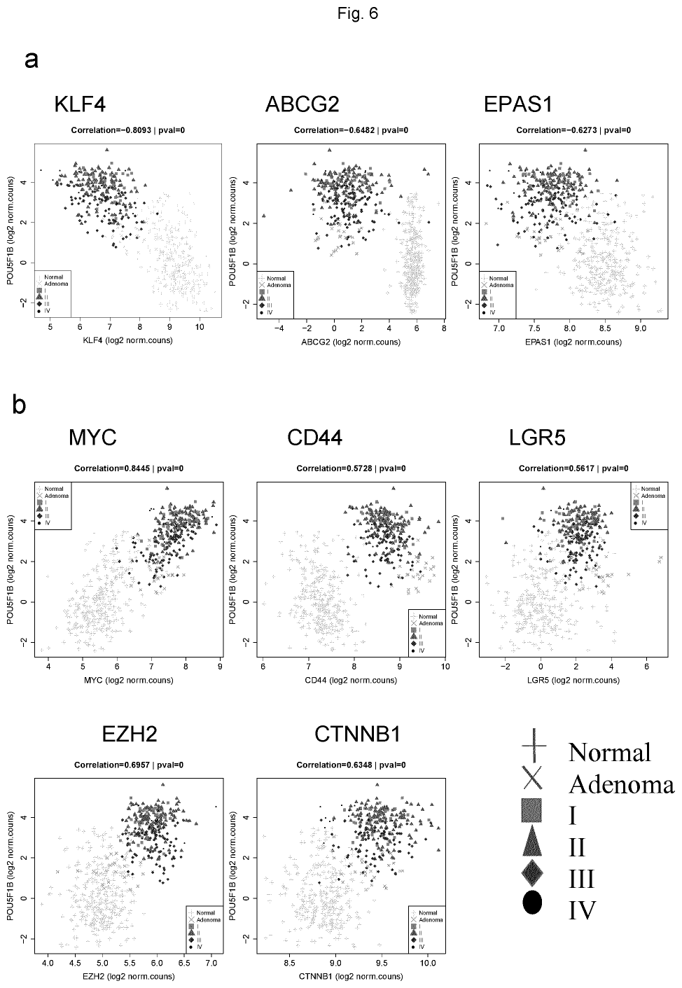

POU5F1B encodes for a transcription factor that is highly similar to POU5F1, also known as OCT4, a major determinant of embryonic stem cell pluripotency. The colony formation assay experiment (FIG. 5), based on the ability of a single cell to transform into a single colony, prompted us to explore the possible participation of POU5F1B in CRC stemness^{[9]}. Taking advantage of recently published mRNA stemness indices^{[10]}, we examined the correlation of POU5F1B expression with stemness markers in CRC. It was recently revealed that expression of KLF4, ABCG2 and EPAS2 anticorrelate with CRC mRNA stemness indices, which conversely correlate with the expression of MYC, CD44, LGR5, EZH2 and CTNNB1^{[10]}. We found POU5F1B transcripts levels to mimic those of cancer stemness indicators in our cohort of 300 CRC patients (FIGS. 6a and 6b). Therefore, POU5F1B displays the features of a colon cancer stem cell marker.

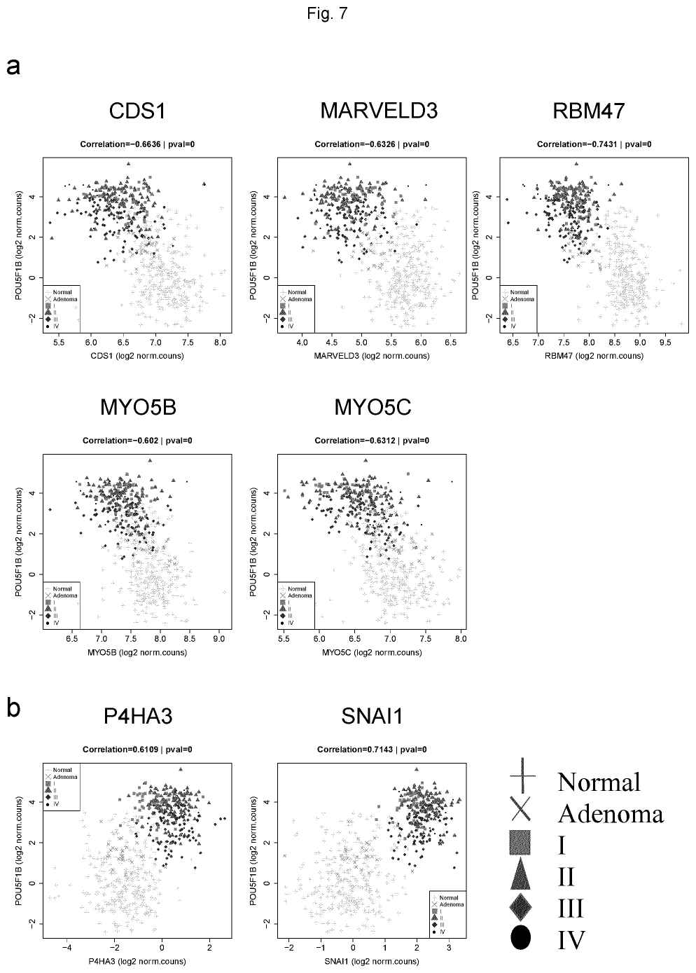

It has been widely reported that cancer stem cells (CSCs) have a high potential to metastasize and, owing to their resistance to chemo- and radiotherapy, they often cause relapse after treatment^{[11]}. Considering the potential role of POU5F1B in CRC stemness and the strong association between the POU5F1B TcGTs and a decrease in relapse-free survival in CRC patients (FIG. 4), we aimed to study a possible link between POU5F1B and metastasis. In cancer progression, the initiation of metastasis occurs through the so-called epithelial-mesenchymal transition (EMT). EMT is a process by which epithelial cells lose their cell polarity and cell-cell adhesion, and gain migratory and invasive properties to become mesenchymal stem cells, which are multipotent stromal cells that can differentiate into a variety of cell types. We looked at the correlation of expression of POU5F1B with several epithelial and mesenchymal markers (FIG. 7) and we found that POU5F1B expression anticorrelates with the expression of epithelial genes and correlates with the expression of mesenchymal genes, suggesting a role for POU5F1B in the mesenchymal transformation and, thus, in the metastatic process.

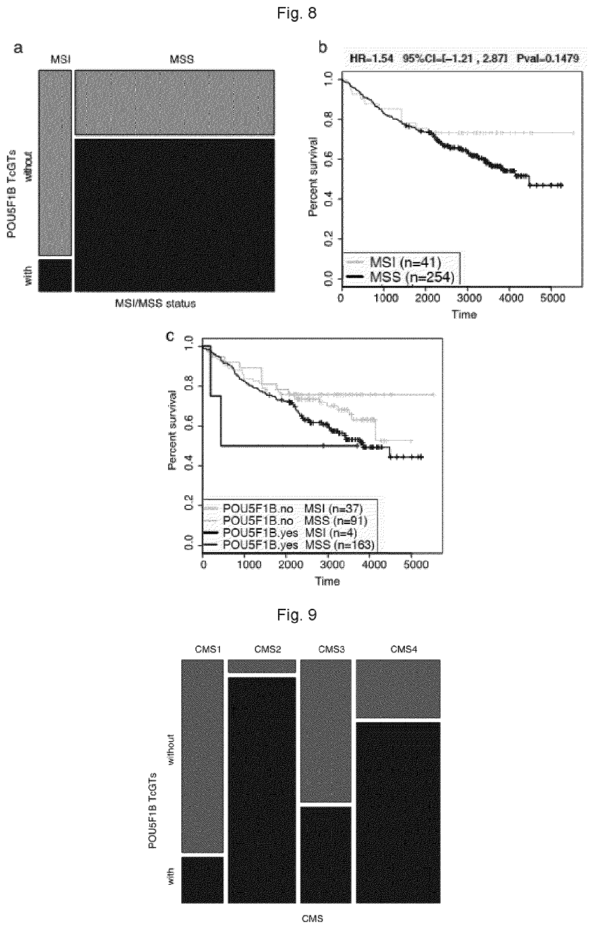

Colorectal cancers (CRCs) can be classified as cancers with microsatellite instability (MSI), which account for 10-20% of all CRC cases and are associated with a better prognosis; and cancers with chromosomal instability but not MSI (MSS), which are associated with worse prognosis^{[12]}. We found the POU5F1B TcGTs more frequently expressed amongst MSS than MSI patients (FIG. 8a). However, the presence of POU5F1B TcGTs had a better predictive power (FIG. 3) than MSI or MSS phenotype (FIG. 8b) (HR=1.69 and pval=0.0095 against HR=1.54 and Pval=0.148 for MSI/MSS).

We compared the predictive power of both markers (POU5F1B or MSI/MSS) by making them compete. To do so, we fit an additive Cox proportional hazards model with both POU5F1B transpochimera and MSI/MSS as predictive variables. The results showed that POU5F1B TcGT keeps a good predictive power (HR=1.45, pval=0.074) when used together with MSS, whereas MSS/MSI loses its predictive power (HR=1.23, pval=0.54) when used together with the POU5F1B transpochimera (FIG. 8c). Therefore, the POU5F1B transpochimera is more informative than MSS/MSI in predicting survival and can be used as an independent predictor.

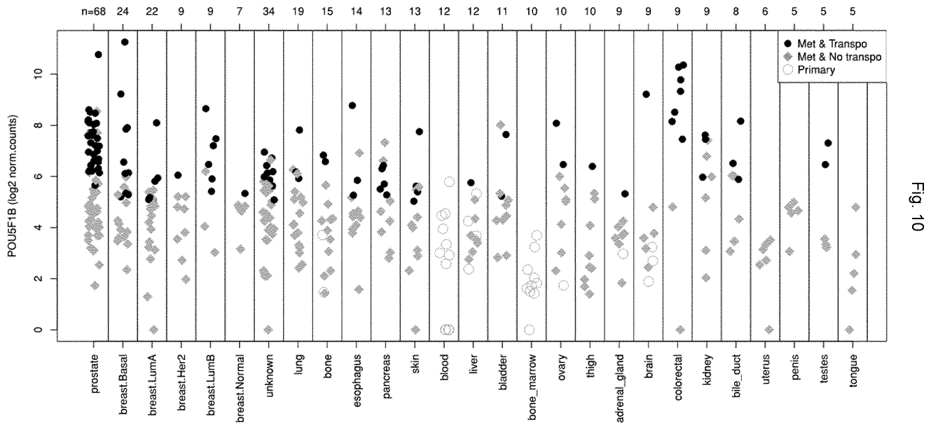

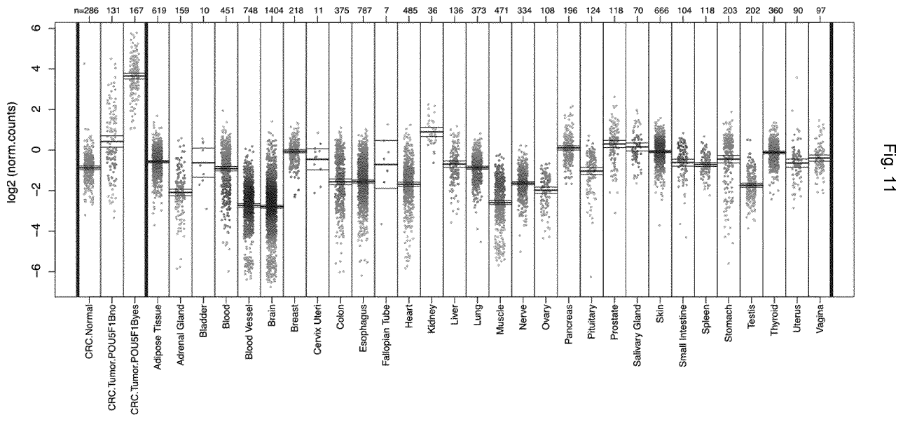

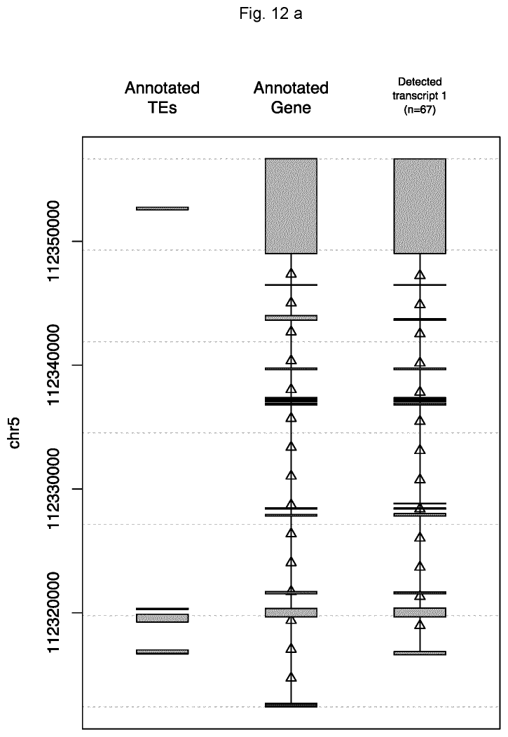

More recently, the CRC Subtyping Consortium (CRCSC) integrated and confronted gene expression-based CRC subtyping together with other data sources (mutation, copy number, methylation, microRNA and proteomics) in order to create four consensus molecular subtypes (CMSs) of CRC: CMS1 (microsatellite instability immune, 14%), hypermutated, microsatellite unstable and strong immune activation; CMS2 (canonical, 37%), epithelial, marked WNT and MYC signaling activation; CMS3 (metabolic, 13%), epithelial and evident metabolic dysregulation; and CMS4 (mesenchymal, 23%), prominent transforming growth factor-beta activation, stromal invasion and angiogenesis^{[13]}. POU5F1B transpochimeric transcripts are expressed in all CMS groups (FIG. 9), suggesting that they are biomarkers of a newly defined subgroup of patients independent from the CMS classification.

Although the POU5F1B gene has been linked to gastric, prostate, liver and breast cancer⁵⁻⁸, its implication in human cancer has not been formally studied. By examining data obtained from a cohort of 500 metastatic lesions originating from different primary tumor sites^{[14]}, we detected POU5F1B-related transpochimeric transcripts not only in CRC, but also in metastatic tissue of other primary tumors including prostate, breast, ovary and testis (FIG. 10).

Since POU5F1B seemed to be a potential target for the treatment of CRC and other primary tumors, targeting the mRNAs or the corresponding translated protein is a promising therapeutic approach. In order to avoid as much as possible drug related side effects, it is very important to know which is the expression of this gene throughout human normal tissues. In FIG. 11 we plotted the expression of POU5F1B gene in the normal colon samples coming from the 300 CRC patients cohort, in the CRC primary tumors not expressing POU5F1B transpochimeric transcripts, in the CRC primary tumors expressing them, and in all the rest of human normal tissues. The expression of POU5F1B is clearly higher in the CRC primary tumors expressing POU5F1B transpochimeric transcripts compared to the rest of samples (FIG. 11), which points out that anti-POU5F1B therapy could be a secure option.

### REFERENCES

1. Lamprecht B, Walter K, Kreher S, Kumar R, Hummel M, Lenze D, Köchert K, Bouhlel M A, Richter J, Soler E, Stadhouders R, Johrens K, Wurster K D, Callen D F, Harte M F, Giefing M, Barlow R, Stein H, Anagnostopoulos I, Janz M, Cockerill P N, Siebert R, Dörken B, Bonifer C, Mathas S. Derepression of an endogenous long terminal repeat activates the CSF1R proto-oncogene in human lymphoma. Nat Med. 2010; 16(5):571-9.
2. Lock F E, Rebollo R, Miceli-Royer K, Gagnier L, Kuah S, Babaian A, Sistiaga-Poveda M, Lai C B, Nemirovsky O, Serrano I, Steidl C, Karimi M M, Mager D L. Distinct isoform of FABP7 revealed by screening for retroelement-activated genes in diffuse large B-cell lymphoma. Proc Natl Acad Sci USA. 2014; 111(34):E3534-43.
3. Babaian A, Romanish M T, Gagnier L, Kuo L Y, Karimi M M, Steidl C, Mager D L. Onco-exaptation of an endogenous retroviral LTR drives IRF5 expression in Hodgkin lymphoma. Oncogene. 2016; 35(19):2542-6.
4. Lock F E, Babaian A, Zhang Y, Gagnier L, Kuah S, Weberling A, Karimi M M, Mager D L. A novel isoform of IL-33 revealed by screening for transposable element promoted genes in human colorectal cancer. PLoS One. 2017; 12(7):e0180659.
5. Hayashi H, Arao T, Togashi Y, Kato H, Fujita Y, De Velasco M A, Kimura H, Matsumoto K, Tanaka K, Okamoto I, Ito A, Yamada Y, Nakagawa K, Nishio K. The OCT4 pseudogene POU5F1B is amplified and promotes an aggressive phenotype in gastric cancer. Oncogene. 2015; 34(2): 199-208.
6. Breyer J P, Dorset D C, Clark T A, Bradley K M, Wahlfors T A, McReynolds K M, Maynard W H, Chang S S, Cookson M S, Smith J A, Schleutker J, Dupont W D, Smith J R. An expressed retrogene of the master embryonic stem cell gene POU5F1 is associated with prostate cancer susceptibility. Am J Hum Genet. 2014; 94(3):395-404.
7. Pan Y, Zhan L, Chen L, Zhang H, Sun C, Xing C. POU5F1B promotes hepatocellular carcinoma proliferation by activating AKT. Biomed Pharmacother. 2018; 100:374-380.
8. Zhao F Q, Misra Y, Li D B, Wadsworth M P, Krag D, Weaver D, Tessitore J, Li D W, Zhang G, Tian Q, Buss K. Differential expression of Oct3/4 in human breast cancer and normal tissues. Int J Oncol. 2018 doi: 10.3892/ijo.2018.4341.
9. Manandhar S, Kim C G, Lee S H, Kang S H, Basnet N, Lee Y M. Exostosin 1 regulates cancer cell stemness in doxorubicin-resistant breast cancer cells. Oncotarget. 2017; 8(41):70521-70537.
10. Malta T M, Sokolov A, Gentles A J, Burzykowski T, Poisson L, Weinstein J N, Kamińska B, Huelsken J, Omberg L, Gevaert O, Colaprico A, Czerwiliska P, Mazurek S, Mishra L, Heyn H, Krasnitz A, Godwin A K, Lazar A J; Cancer Genome Atlas Research Network, Stuart J M, Hoadley K A, Laird P W, Noushmehr H, Wiznerowicz M. Machine Learning Identifies Stemness Features Associated with Oncogenic Dedifferentiation. Cell. 2018; 173(2):338-354.e15.
11. Nguyen L V, Vanner R, Dirks P, Eaves C J. Cancer stem cells: an evolving concept. Nat Rev Cancer. 2012; 12(2):133-43.
12. Kim C G, Ahn J B, Jung M, Beom S H, Kim C, Kim J H, Heo S J, Park H S, Kim J H, Kim N K, Min B S, Kim H, Koom W S, Shin S J. Effects of microsatellite instability on recurrence patterns and outcomes in colorectal cancers. Br J Cancer. 2016; 115(1):25-33.
13. Guinney J, Dienstmann R, Wang X, de Reyniés A, Schlicker A, Soneson C, Marisa L, Roepman P, Nyamundanda G, Angelino P, Bot B M, Morris J S, Simon I M, Gerster S, Fessler E, De Sousa E Melo F, Missiaglia E, Ramay H, Barras D, Homicsko K, Maru D, Manyam G C, Broom B, Boige V, Perez-Villamil B, Laderas T, Salazar R, Gray J W, Hanahan D, Tabernero J, Bernards R, Friend S H, Laurent-Puig P, Medema J P, Sadanandam A, Wessels L, Delorenzi M, Kopetz S, Vermeulen L, Tejpar S. The consensus molecular subtypes of colorectal cancer. Nat Med. 2015; 21(11):1350-6.
14. Robinson D R, Wu Y M, Lonigro R J, Vats P, Cobain E, Everett J, Cao X, Rabban E, Kumar-Sinha C, Raymond V, Schuetze S, Alva A, Siddiqui J, Chugh R, Worden F, Zalupski M M, Innis J, Mody R J Tomlins S A, Lucas D, Baker L H, Ramnath N, Schott A F, Hayes D F, Vijai J, Offit K, Stoffel E M, Roberts J S, Smith D C, Kunju L P, Talpaz M, Cieślik M, Chinnaiyan A M. Integrative clinical genomics of metastatic cancer. Nature. 2017; 548(7667):297-303.

1. A method for detecting and/or predicting the likelihood of a cancer in a subject, the method comprising detecting, directly or indirectly, in a biological sample of said subject, the presence or absence of at least one nucleic acid sequence comprising
i) at least one transposable element (TE), a fragment or variant thereof, and
ii) at least one gene sequence, a fragment or variant thereof,
wherein said at least one gene sequence is selected from the group listed in Table 1, or a variant or fragment thereof.

2. The method of claim 1, wherein the gene sequence is a POU5F1B sequence, fragment or variant thereof.

3. The method of claim 1, wherein the nucleic acid sequence is an RNA transcript, a cDNA of said RNA transcript or a DNA sequence.

4. The method of claim 1, wherein the at least one TE is a long terminal repeat (LTR) of an endogenous retrovirus.

5. The method of claim 1, wherein the RNA transcript is a transpochimeric gene transcript (TcGT).

6. The method of claim 1, wherein the cancer is a solid tumor cancer selected from the group consisting of Colorectal Cancer, Gastric Cancer, Prostate Cancer, Liver Cancer, Testis Cancer, Ovary Cancer, Breast Cancer, and a combination of one of more thereof.

7. The method of claim 6, wherein the cancer is Colorectal Cancer.

8. The method of claim 1, wherein the at least one acid nucleic sequence is selected from the group consisting of SEQ ID No 1, 2, 3, 4, 5, 6, 7, 8, 9, 10, 11, 12, 13, 14, 15, 16, 17, 18, 19, 20, 21, 22, 23, 24, 25, 26, 27, and a combination of one or more thereof.

9. A method for classifying a cancer subject, the method comprising
i) detecting, directly or indirectly, in a biological sample of said subject, the presence or absence of at least one nucleic acid sequence comprising at least one transposable element (TE) or a fragment or variant thereof, and at least one gene sequence, a fragment or variant thereof, wherein said at least one gene sequence is selected from the group listed in Table 1 or a variant or fragment thereof; and
ii) classifying the cancer subject
as having a favourable prognosis based on the absence of the at least one nucleic acid sequence in the biological sample; or
as having a poor prognosis based on the presence of the at least one nucleic acid sequence in the biological.

10. The method of claim 9, wherein the at least one gene sequence is selected from the group consisting of POU5F1B, FAAH2, MBD5, ME3, NXPE4, PKP1, PLCB1, SNX1, DCP2, and YIPF4, a fragment or variant thereof, and a combination of one of more thereof.

11. The method of claim 10, wherein the gene sequence is a POU5F1B sequence, a fragment or a variant thereof.

12. The method of claim 9, wherein the at least one TE is a long terminal repeat (LTR) of an endogenous retrovirus.

13. The method of claim 9, wherein the at least one nucleic acid is an RNA transcript.

14. The method of claim 9, wherein the cancer is a solid tumor cancer selected from the group consisting of Colorectal Cancer, Gastric Cancer, Prostate Cancer, Liver Cancer, Testis Cancer, Ovary Cancer, Breast Cancer, and a combination of one of more thereof.

15. The method of claim 9, wherein the cancer is Colorectal Cancer.

16. A method for classifying a cancer subject, the method comprising
1) detecting, directly or indirectly, in a biological sample of said subject, the presence or absence of
at least one nucleic acid sequence correlating with an increase of survival (POSITIVE PROGNOSIS), said nucleic acid sequence comprising at least one transposable element (TE) a fragment or variant thereof, and at least one gene sequence selected from the group consisting of DCP2, YIPF4, and a fragment or variant thereof; and
at least one nucleic acid sequence correlating with a decrease of survival (NEGATIVE PROGNOSIS), said nucleic acid sequence comprising at least one transposable element (TE), a fragment or variant thereof, and at least one gene sequence selected from the group consisting of POU5F1B, FAAH2, MBD5, ME3, NXPE4, PKP1, PLCB1, SNX1, and a fragment or variant thereof; and
2) scoring the number of nucleic acid sequences correlating with an increase of survival (POSITIVE PROGNOSIS) and the number of nucleic acid sequences correlating with a decrease of survival (NEGATIVE PROGNOSIS);
wherein the
number of protective events>number of harmful events is indicative of increase in survival (POSITIVE PROGNOSIS); and
number of harmful events>number of protective events is indicative of decrease in survival (NEGATIVE PROGNOSIS).

17. The method of claim 16, wherein the cancer is a solid tumor cancer selected from the group consisting of Colorectal Cancer, Gastric Cancer, Prostate Cancer, Liver Cancer, Testis Cancer, Ovary Cancer, Breast Cancer, and a combination of one of more thereof.

18. The method of claim 16, wherein the at least one TE is a long terminal repeat (LTR) of an endogenous retrovirus.

19. The method of claim 16, wherein the cancer is Colorectal Cancer.

20. A method for staging a cancer in a subject, the method comprising measuring, directly or indirectly, in a biological sample of said subject, the level of expression or activity of at least one nucleic acid sequence and comparing said level of expression or activity to a control value or range, wherein said at least one nucleic acid sequence comprises
i) at least one transposable element (TE), or a fragment or variant thereof, and
ii) at least one gene sequence, or a fragment or variant thereof,
and wherein said at least one gene sequence is selected from the group listed in Table 1, or a variant or fragment thereof.

21. The method of claim 20, wherein the cancer is a solid tumor cancer selected from the group consisting of Colorectal Cancer, Gastric Cancer, Prostate Cancer, Liver Cancer, Testis Cancer, Ovary Cancer, Breast Cancer, and a combination of one of more thereof.

22. The method of claim 20, wherein the at least one TE is a long terminal repeat (LTR) of an endogenous retrovirus.

23. The method of claim 20, wherein the cancer is Colorectal Cancer.

24.-51. (canceled)
